(19) United States
(12) Patent Application Publication
(10) Pub. No.: US 2022/0235418 A1
(43) Pub. Date: Jul. 28, 2022

(54) Use of Biomarkers for Degenerative Disc Disease

(71) Applicant: Predictive Technology Group, Inc., Salt Lake City, UT (US)

(72) Inventors: Rakesh N. CHETTIER, West Jordan, UT (US); Kenneth WARD, Salt Lake City, UT (US)

(21) Appl. No.: 17/605,295

(22) PCT Filed: Apr. 22, 2020
(86) PCT No.: PCT/US2020/029357

§ 371 Date: Oct. 21, 2021

Related U.S. Application Data
(60) Provisional application No. 62/836,885, filed on Apr. 22, 2019.

(51) Int. Cl. C12Q 1/6883; G16H 50/30; G16H 15/00; G16H 10/40

(52) U.S. Cl. CPC C12Q 1/6883; G16H 50/30; C12Q 2600/118; G16H 10/40; C12Q 2600/156; G16H 15/00

(57) ABSTRACT

Disclosed herein are methods of using genetic markers associated with degenerative disc disease, for example via a computer-implemented program to predict risk of developing degenerative disc disease, and methods of preventing or treating degenerative disc disease or a symptom thereof.

### CROSS REFERENCE

This application claims the benefit of U.S. Provisional Application No. 62/836,885, filed Apr. 22, 2019, which is incorporated by reference herein in its entirety.

### BRIEF SUMMARY

The methods and systems described herein provide an approach for sequencing a nucleic acid sample using high throughput methods to detect genetic variants. These methods provide improved methods in the field of diagnosis, assessment and treatment of degenerative disc disease. For example, disclosed herein is the use of nanopore sequencing to detect one or more genetic variants in a nucleic acid sample, wherein the one or more genetic variants are listed in FIG. 1.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “figure” and “FIG.” herein), of which:

FIG. 1 shows a table of single nucleotide polymorphisms (SNPs).

FIG. 2 is a diagram showing a method and system as disclosed herein.

FIG. 3 shows a computer control system that is programmed or otherwise configured to implement methods provided herein.

### DETAILED DESCRIPTION

Degenerative Disc Disease (DDD) may be understood to include other related spine diseases such as lumbar disc disease (LDD). DDD may be a disease that may be characterized by the loss of moisture in or dehydration of one or more discs in the spine and may further be characterized by the loss of capacity of the one or more discs to function as shock absorbers between one or more vertebrae of the spine. DDD is a leading cause of medical disability in the United States and throughout the world. DDD may have a major impact on over-all health care costs, productivity and the quality of life for many individuals. Progression or initiation of DDD may be related to a spinal injury event. In other cases, progression or initiation of DDD may be related to familial clustering often observed among DDD subjects—familial clustering that may be due in part to a genetic influence. In some cases, greater than about 63% of severe lumbar and cervical spine changes may be heritable. Specific genes have been implicated in the pathogenesis of DDD. However, a comprehensive search for polymorphisms and other genetic markers associated with, predictive of, or diagnostic of DDD has not been performed.

Onset or progression of DDD may be caused by a drying of one or more discs, a tear or physical damage to one or more discs, or a combination thereof. In some cases, a healthy disc or a disc absent DDD may comprise about 80% water. In some cases, a healthy disc or a disc absent DDD may comprise from about 75% to about 85% water. In some cases, a healthy disc or a disc absent DDD may comprise from about 76% to about 84% water. In some cases, a healthy disc or a disc absent DDD may comprise from about 77% to about 83% water.

DDD onset or progression may occur when a disc comprises less than 80% water. For example, DDD onset or progression may occur when a disc comprises from about 75% to about 1% water. DDD onset or progression may occur when a disc comprises from about 70% to about 1% water. DDD onset or progression may occur when a disc comprises from about 65% to about 1% water. DDD onset or progression may occur when a disc comprises from about 60% to about 1% water. DDD onset or progression may occur when a disc comprises from about 55% to about 1% water. DDD onset or progression may occur when a disc comprises from about 50% to about 1% water. DDD onset or progression may occur when a disc comprises from about 45% to about 1% water. DDD onset or progression may occur when a disc comprises from about 40% to about 1% water. DDD onset or progression may occur when a disc comprises from about 35% to about 1% water. DDD onset or progression may occur when a disc comprises from about 30% to about 1% water. DDD onset or progression may occur when a disc comprises from about 25% to about 1% water. DDD onset or progression may occur when a disc comprises from about 20% to about 1% water.

DDD onset or progression may occur when a disc comprises from about 75% to about 10% water. DDD onset or progression may occur when a disc comprises from about 70% to about 10% water. DDD onset or progression may occur when a disc comprises from about 65% to about 10% water. DDD onset or progression may occur when a disc comprises from about 60% to about 10% water. DDD onset or progression may occur when a disc comprises from about 55% to about 10% water. DDD onset or progression may occur when a disc comprises from about 50% to about 10% water. DDD onset or progression may occur when a disc comprises from about 45% to about 10% water. DDD onset or progression may occur when a disc comprises from about 40% to about 10% water. DDD onset or progression may occur when a disc comprises from about 35% to about 10% water. DDD onset or progression may occur when a disc comprises from about 30% to about 10% water. DDD onset or progression may occur when a disc comprises from about 25% to about 10% water. DDD onset or progression may occur when a disc comprises from about 20% to about 10% water.

DDD onset or progression may be caused by a physical damage to a disc. A physical damage may comprise a tear, such as a tear in a portion of an outer wall of a disc. A physical damage may be a result of a physical activity or an injury. A physical damage to a disc may result in an inner portion of the disc to bulge or push through the outer wall. A physical damage to a disc may result in a physical displacement of the disc from its normal or initial position.

A subject having DDD may be asymptomatic. A subject having DDD may be symptomatic. A subject having DDD may suffer pain. The pain may be intermittent pain. The pain may be constant pain. The pain may be more severe when sitting, bending, lifting an object, or twisting. The pain may not be more severe in these cases. The pain may be less severe when changing a position or when lying down. The pain may not be less severe in these cases. Pain may be localized, such as for example the lower back, the upper back, the buttocks, the thighs, or any combination thereof.

The methods as described herein may provide a method of diagnosing a subject as having DDD. The methods may include identifying a subject at risk of developing DDD. The diagnosis may also include additional elements. For example, a diagnosis of DDD may include a method as described herein alone or in combination with: a review of a subject's medical history, a subject's family history of other family members diagnosed with DDD or having symptoms of DDD, a physical examination, symptoms of DDD (such as intermittent pain), an imaging procedure (such as an MRI) to confirm a presence of damage to one or more discs, or any combination thereof.

A subject at risk of developing DDD or confirmed to have DDD may be treated. A treatment may include treating the symptoms associated with DDD, supporting or replacing the disc having the DDD, or a combination thereof. Treatment may include replacing a disc having DDD with an artificial disc replacement. Treatment may include supporting a disc having DDD by having a subject wear a back brace or an internal or external bracing device. Treatment may include physical therapy to strengthen surrounding muscles or ligaments structures adjacent to or distal to the affected disc. Treatment may include pain management in the form of acupuncture, massage, physical therapy, or a combination thereof. Treatment may include pain management in the form of administering a medical composition to the subject, such as an opioid, a muscle relaxant, an NSAID, or other pain-reducer. Treatment may include reducing inflammation adjacent to the affected disc, by administering to the subject injections, such as injections of steroid proximal to the affected disc.

Provided herein are methods for detecting a presence of one or more polymorphisms, such as single nucleotide polymorphisms (SNP) in genetic material from a subject. The genetic material may be obtained from a sample, such as a blood sample, a cell-free sample, a buccal swab (such as a swab from a portion of the oral cavity, such as a mouth or throat), a nasal swab, a sample comprising saliva, a fine needle aspirate sample or other. Genetic material may include DNA, RNA, protein, or a combination thereof. The subject providing the genetic material may have or be suspected of having or at risk of developing DDD. The one or more polymorphisms to be detected in a genetic material may be selected based on an odds ratio of being indicative of DDD. The one or more polymorphism to be detected may be selected based on feature selection of a trained algorithm that has been trained with genetic material from subjects having DDD, genetic material from healthy subjects, or a combination thereof.

Data or information obtained from a medical questionaire or medical history from a subject, from an assay (such as a genetic assay or a non-genetic assay), or any combination thereof may be input to a trained algorithm. A trained algorithm may include a nearest neighbor algorithm, a random forest algorithm, a support vector machine (SVM) algorithm, a decision tree algorithm, a linear regression algorithm, a logistic regression algorithm, a naive bayes algorithm, a kNN algorithm, any combination thereof or others. A trained algorithm may utilize feature selection to identify one or more SNPs predictive of a presence or an absence of DDD, predictive of a risk of developing DDD, predictive of an efficacy of a treatment for DDD, predictive of a recurrence of DDD or any combination thereof. A trained algorithm may rank or weight one or more SNPs. A highly ranked or highly weighted SNP may provide an identification of a presence or an absence of DDD in a sample at a greater accuracy than an SNP that may be lower ranked or weighted. A trained algorithm may identify a panel of one or more SNPs that may identify a presence or an absence of DDD at an accuracy of at least 80%, 85%, 90%, 95% or greater. A panel may comprise a single SNP. A panel may comprise a plurality of SNPs. A panel may comprise an SNP in combination with another polymorphism (such as an insertion, a deletion, a variation, a repetitive element, or a copy number change in a polymorphism), or another gene having a differential level of expression compared to a reference. A panel may comprise at least 2, 3, 4, 5, 6, 7, 8, 9, 10, 11, 12, 13, 14, 15, 20, 25, 30, 35, 40, 45, 50, 75, 100, 125, 150, 175, 200, 250 SNPs or more. A panel may comprise at least 5 SNPs. A panel may comprise at least 10 SNPs. A panel may comprise at least 20 SNPs. A panel may comprise at least 50 SNPs. A panel may comprise at least 100 SNPs. A trained algorithm may receive a data set comprising information obtained from a medical questionaire or medical history obtained from a subject, from a genetic-based assay, from a non-genetic based assay, from a cytological assay, from an immuno-based assay, from a sequencing assay, or any combination thereof. A trained algorithm or other computer processing element may output a report, such as a printed report or an electronic report. A report may comprise a result obtained from analyzing the sample. A result may include a presence or absence of DDD in the sample. A report may comprise details of the sample, such as sample type, a collection media, a collection source, a panel of SNPs, genes or other markers assessed. A report may comprise a recommendation, such as a recommendation of treatment or second diagnostic assay based on a result obtained.

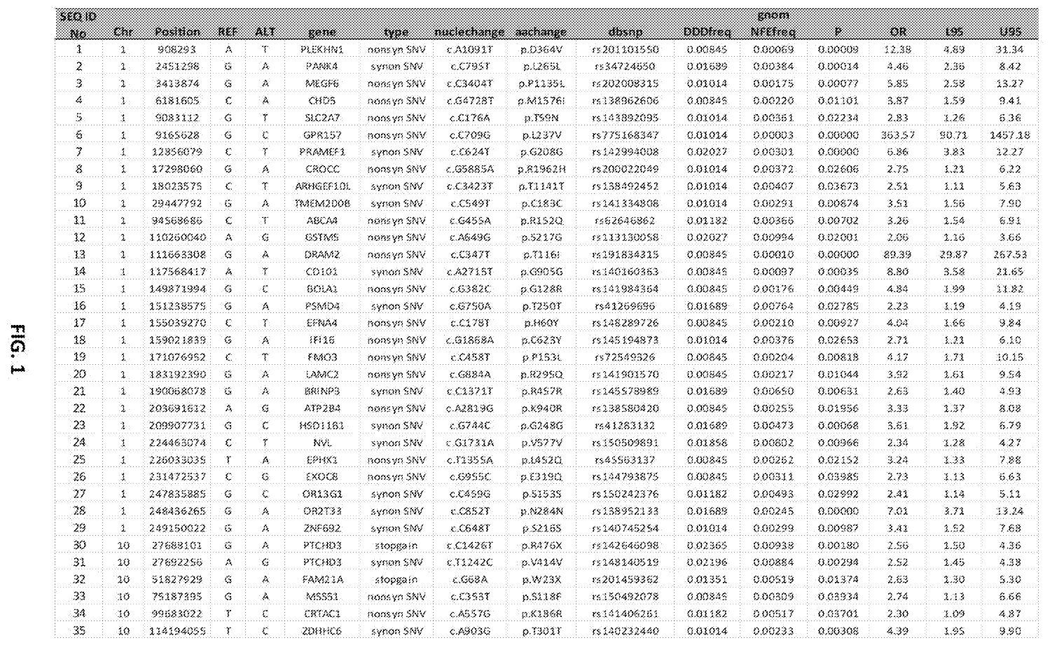

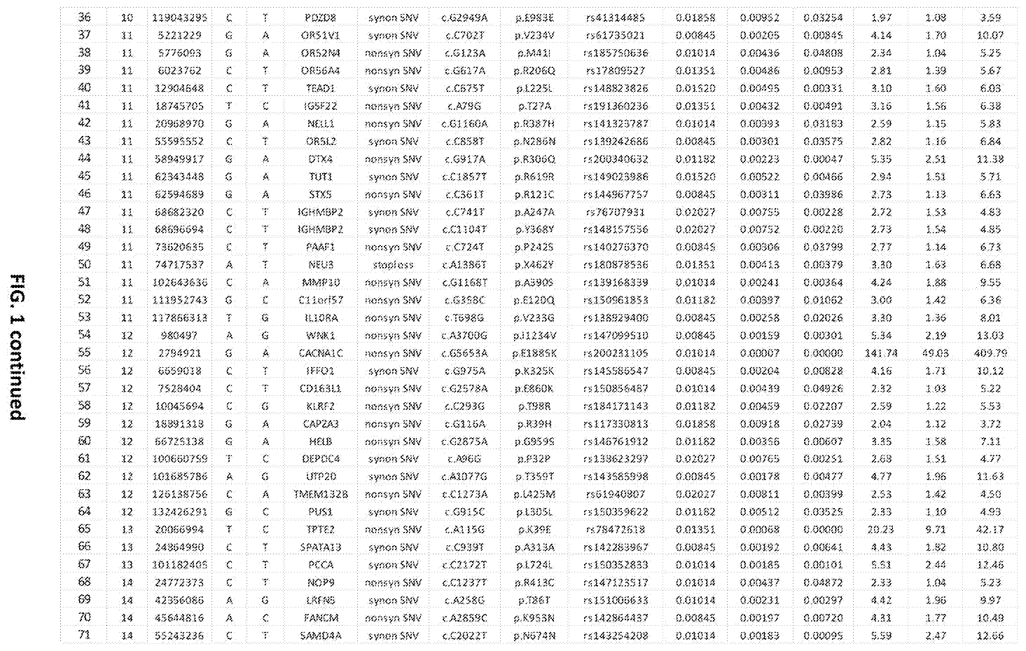

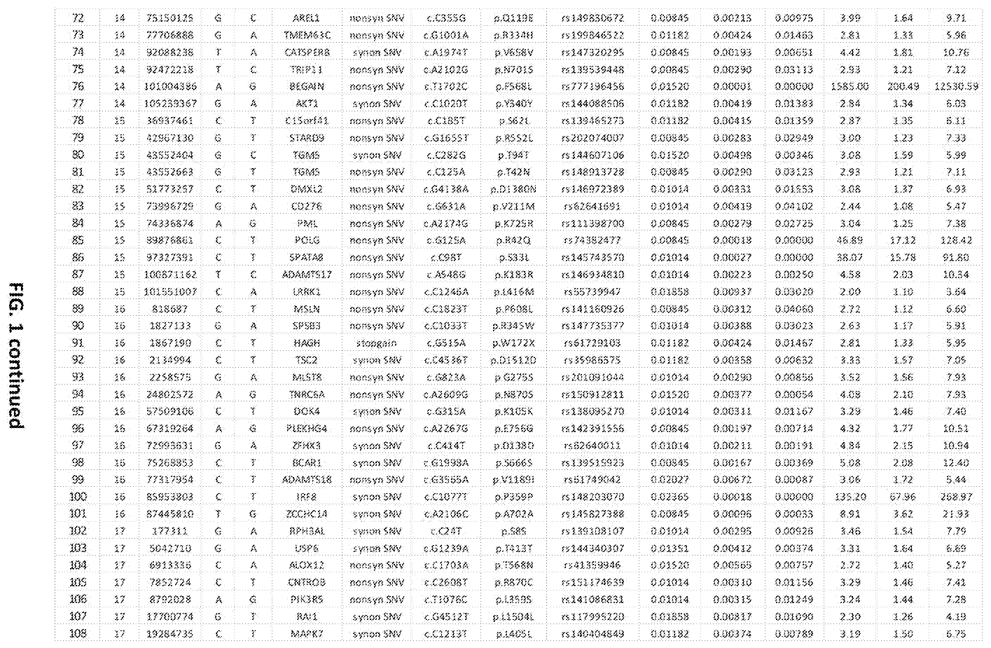

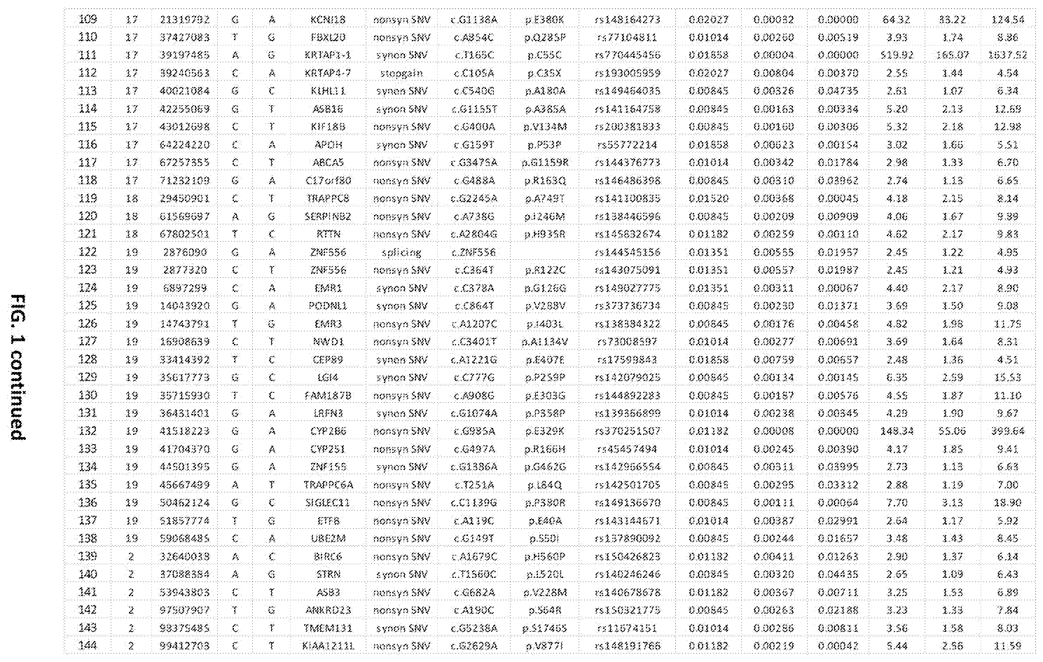

SNPs 1-276 of FIG. 1, or any combination thereof may comprise one or more polymorphisms. A sequence, detected by methods described herein, may comprise said one or more polymorphisms and may have at least about: 80%, 85%, 90%, 95% sequence homology to any one of SNPs 1-276 of FIG. 1. A sequence may comprise said one or more polymorphisms and may have at least about 80% or more sequence homology to any one of SNPs 1-276 of FIG. 1. A sequence may comprise said one or more polymorphisms and may have at least about 85% or more sequence homology to any one of SNPs 1-276 of FIG. 1. A sequence may comprise said one or more polymorphisms and may have at least about 90% or more sequence homology to any one of SNPs 1-276 of FIG. 1. A sequence may comprise said one or more polymorphisms and may have at least about 95% or more sequence homology to any one of SNPs 1-276 of FIG. 1. A sequence may comprise said one or more polymorphisms and may have at least about 96% or more sequence homology to any one of SNPs 1-276 of FIG. 1. A sequence may comprise said one or more polymorphisms and may have at least about 97% or more sequence homology to any one of SNPs 1-276 of FIG. 1. A sequence may comprise said one or more polymorphisms and may have at least about 98% or more sequence homology to any one of SNPs 1-276 of FIG. 1. A sequence may comprise said one or more polymorphisms and may have at least about 99% or more sequence homology to any one of SNPs 1-276 of FIG. 1.

The one or more polymorphisms may comprise any one of SNP 1-SNP 276. The one or more polymorphisms may comprise any one of SNP 6; SNP 76; SNP 111; SNP 212, or any combination thereof. The one or more polymorphisms may comprise any one of SNP 25; SNP 88; SNP 120; SNP 188, or any combination thereof. The one or more polymorphisms may comprise any one of SNP 1; SNP 6; SNP 13; SNP 55; SNP 65; SNP 76; SNP 85; SNP 86; SNP 100; SNP 109; SNP 111; SNP 132; SNP 145; SNP 154; SNP 211; SNP 212, or any combination thereof.

The one or more polymorphisms may comprise any combination of SNPs of FIG. 1 having an odds ratio of greater than about 3. The one or more polymorphisms may comprise any combination of SNPs of FIG. 1 having an odds ratio of greater than about 4. The one or more polymorphisms may comprise any combination of SNPs of FIG. 1 having an odds ratio of greater than about 5. The one or more polymorphisms may comprise any combination of SNPs of FIG. 1 having an odds ratio of greater than about 6. The one or more polymorphisms may comprise any combination of SNPs of FIG. 1 having an odds ratio of greater than about 7. The one or more polymorphisms may comprise any combination of SNPs of FIG. 1 having an odds ratio of greater than about 8. The one or more polymorphisms may comprise any combination of SNPs of FIG. 1 having an odds ratio of greater than about 9. The one or more polymorphisms may comprise any combination of SNPs of FIG. 1 having an odds ratio of greater than about 10.

A portion of the genetic material may be utilized for detection of the one or more polymorphisms and a second portion subjected to a second assay, such as a diagnostic assay. A portion of the genetic material may be assayed and a second portion may be stored for later use.

The disclosure provided herein include a genetic-based test based on DDD associated markers that may provide diagnostic information, prognostic information or a combination thereof. A prognostic test may provide information that may be a basis for a treatment option for the subject such as tissue regeneration of the affected disc or gene-based therapy of the spine. In some case, gene-based therapy may be applied to the lumbar spine before premature degenerative changes occur. In addition, a prognostic test may also assist in the appropriate use of currently available total-disc replacement technologies, by identifying subject populations that would benefit from the treatment. Further, the noted DDD genetic screening test may improve gene-based therapy of DDD, as such an option may be limited to early development of DDD, but also by the lack of a scientific basis to identify appropriate candidates for early intervention of DDD. As additional genes and genetic markers that may be associated with juvenile and young adult DDD may be sequenced, the noted DDD genetic screening test may provide information about the molecular pathways involved in DDD that may lead to innovative pharmacological solutions or recombinant molecules useful in the treatment or prevention of DDD.

In some embodiments, provided herein may be methods for identification of genetic variants (such as SNPs, indels, unique combinations or panels of such genetic variants, haplotypes of genetic variants, or combination thereof) indicative of a presence of or a risk of developing degenerative disc disease (DDD) or related spinal pathologies. In some embodiments, the polymorphisms can be directly useful as targets for the design of diagnostic reagents and the development of therapeutic agents for use in the diagnosis and treatment of DDD and related pathologies. Based on the identification of variants associated with DDD, the present disclosure can provide methods of detecting these variants as well as the design and preparation of detection reagents needed to accomplish this task. Provided herein may be novel variants in genetic sequences involved in DDD, methods of detecting these variants in a test sample, methods of identifying individuals who have an altered risk of developing DDD and for suggesting treatment options for DDD based on the presence of a variant(s) or its encoded product and methods of identifying individuals who may be more or less likely to respond to a treatment.

In some embodiments, provided herein may be variants such as SNPs and indels associated with DDD, nucleic acid molecules containing variants, methods and reagents for the detection of the variants, uses of these variants for the development of detection reagents, and assays or kits that utilize such reagents. In some embodiments, the variants can be useful for diagnosing, screening for, and evaluating predisposition to DDD and progression of DDD. In some embodiments, the variants can be useful in the determining individual subject treatment plans and design of clinical trials of devices for possible use in the treatment of DDD. In some embodiments, the variants and their encoded products can be useful targets for the development of therapeutic agents. In some embodiments, the variants combined with other non-genetic clinical factors can be useful for diagnosing, screening, evaluating predisposition to DDD, assessing risk of progression of DDD, determining individual subject treatment plans and design of clinical trials of devices for possible use in the treatment of DDD.

The present disclosure relates to the identification of novel polymorphisms, unique combinations of such polymorphisms, and haplotypes of polymorphisms that may be associated with DDD and related pathologies. The polymorphisms may be directly useful as targets for the design of diagnostic reagents and the development of therapeutic agents for use in the diagnosis and treatment of DDD and related pathologies.

Based on the identification of particular single nucleotide polymorphisms (SNPs) associated with DDD, the present disclosure also provides methods of detecting these variants as well as the design and preparation of detection reagents needed to accomplish this task. In some embodiments, the disclosure specifically provides novel SNPs in genetic sequences involved in DDD, methods of detecting these SNPs in a test sample, methods of identifying individuals who have an altered risk of developing DDD or for developing progressive DDD based on the presence of a SNP(s) or its encoded product and methods of identifying individuals who may be more or less likely to respond to a treatment. For the purposes of this application, progressive DDD may be understood to mean DDD that progresses at a rate that may be greater than a rate associated with mere aging of a disc of a human.

In some embodiments, the present disclosure provides a method for determining whether a human subject has DDD or may be at risk of developing DDD, comprising: detecting in the genetic material of said subject the presence or absence of one or more protective or high-risk polymorphism selected from the group consisting of the polymorphisms of FIG. 1 or a polymorphism that may be in linkage disequilibrium with a polymorphism of FIG. 1, wherein the polymorphism may be correlated with DDD or an altered risk of developing DDD.

In one embodiment of the disclosure, the present disclosure provides polymorphisms having significant allelic association with DDD, as set forth in FIG. 1 or polymorphisms that may be in linkage disequilibrium with a polymorphism of FIG. 1. In some embodiments, the polymorphisms may be selected from the polymorphisms of FIG. 1. FIG. 1 provides information identifying the SNPs of the present disclosure, including SNP “rs” identification numbers (a reference SNP or RefSNP accession ID number), P values, and chromosome number.

In some embodiments, naturally-occurring SNPs in the human genome may be provided that are associated with DDD. Such SNPs can have a variety of uses in the diagnosis and/or treatment of DDD. One aspect relates to an isolated nucleic acid molecule comprising a nucleotide sequence in which at least one nucleotide may be a SNP disclosed in FIG. 1. In some embodiments, a nucleic acid of the disclosure can be an amplified polynucleotide, which may be produced by amplification of a SNP-containing nucleic acid template.

In some embodiments, a reagent for detecting a SNP in the context of its naturally-occurring flanking nucleotide sequences (which can be, e.g., either DNA or mRNA) may be provided. In some embodiments, a reagent may be in the form of a hybridization probe or an amplification primer that may be utilized in detection of a SNP of interest.

A primer may comprise a sequence having sequence complementarity to at least a portion of any one of SNP 1-SNP 276. A primer may comprise a sequence having at least 80%, 85%, 90%, 95% sequence complementarity to any one of SNP 1-SNP 276. A primer may comprise a sequence having at least 80% sequence complementarity to any one of SNP 1-SNP 276. A primer may comprise a sequence having at least 85% sequence complementarity to any one of SNP 1-SNP 276. A primer may comprise a sequence having at least 90% sequence complementarity to any one of SNP 1-SNP 276. A primer may comprise a sequence having at least 95% sequence complementarity to any one of SNP 1-SNP 276. A primer may comprise a sequence having at least 96% sequence complementarity to any one of SNP 1-SNP 276. A primer may comprise a sequence having at least 97% sequence complementarity to any one of SNP 1-SNP 276. A primer may comprise a sequence having at least 98% sequence complementarity to any one of SNP 1-SNP 276. A primer may comprise a sequence having at least 99% sequence complementarity to any one of SNP 1-SNP 276.

Also provided in the disclosure may be kits comprising SNP detection reagents and methods for detecting the SNPs by employing detection reagents. In some embodiments, the present disclosure provides for a method of identifying an individual having an increased or decreased risk of developing DDD by detecting the presence or absence of a SNP allele. In some embodiments, a method for diagnosis of DDD by detecting the presence or absence of a SNP allele is provided.

In some embodiments, the disclosure also provides a kit. A kit may comprise SNP detection reagents. For example, a kit may include one or more primer sequences for detecting one or more SNPs, such as one or more SNPs of FIG. 1. A primer may comprise a sequence having at least 80% sequence complementarity to at least a portion of any one of SNP 1 through SNP 276 of FIG. 1. A kit may include one or more containers. Each primer may be housed in a separate container. One or more primers may be housed in a single container. A kit may include a control sample or a reference sample. The control sample or reference sample may comprise a sequence having one or more SNPs. The control or reference sample may comprise a sequence having at least 80% sequence homology to one or more of SNP 1 through SNP 276 of FIG. 1. A kit may comprise instructions for performing methods of detecting the SNPs by employing detection reagents or performing an analyzing such as inputting data into a trained algorithm and obtained a result. A kit may comprise a questionnaire or medical history of non-genetic clinical factors. In some embodiments, the questionnaire may be completed by subject or by another individual (such as a medical professional, including a social worker, health worker, doctor, technician, medical intake professional). The questionnaire may provide values for various criteria including the number of herniated discs, sciatica episodes, decreased disc height, dark nucleus pulposus and the Schneiderman or Pfirrmann grade which evaluates signal changes within the nucleus pulposus of the intervertebral discs of the lumbar spine. In some embodiments, the questionnaire may include any other non-genetic clinical factors associated with the risk of developing DDD or the risk for progressive DDD.

Many other uses and advantages may be apparent to those skilled in the art upon review of the detailed description of the embodiments herein. Solely for clarity of discussion, the disclosure may be described in the sections below by way of non-limiting examples.

### Definitions

Unless otherwise indicated, open terms for example “contain,” “containing,” “include,” “including,” and the like mean comprising.

The singular forms “a”, “an”, and “the” are used herein to include plural references unless the context clearly dictates otherwise. Accordingly, unless the contrary is indicated, the numerical parameters set forth in this application are approximations that may vary depending upon the desired properties sought to be obtained by the present disclosure.

Unless otherwise indicated, some instances herein contemplate numerical ranges. When a numerical range is provided, unless otherwise indicated, the range includes the range endpoints. Unless otherwise indicated, numerical ranges include all values and subranges therein as if explicitly written out. Unless otherwise indicated, any numerical ranges and/or values herein, following or not following the term “about,” can be at 85-115% (i.e., plus or minus 15%) of the numerical ranges and/or values.

As used herein, “treatment” may include one or more of: reducing the frequency and/or severity of symptoms, elimination of symptoms and/or their underlying cause, and improvement or remediation of damage. For example, treatment of degenerative disc disease may include relieving the pain experienced by a person suffering from DDD.

As used herein, a “therapeutic” can include a medical device, a pharmaceutical composition, a medical procedure, or any combination thereof. In some embodiments, a medical device may comprise a spinal brace. In some embodiments a medical device may comprise an artificial disc device. A medical device may comprise a surgical implant, a disc replacement or a disc transplantation. A pharmaceutical composition may comprise a muscle relaxant, an anti-depressant, a steroid, an opioid, a cannabis-based therapeutic, acetaminophen, a non-steroidal anti-inflammatory, a neuropathic agent, a cannabis, a progestin, a progesterone, or any combination thereof. A neuropathic agent may comprise gabapentin. A non-steroidal anti-inflammatory may comprise naproxen, ibuprofen, a COX-2 inhibitor, or any combination thereof. A pharmaceutical composition may comprises a biologic agent, cellular therapy, regenerative medicine therapy, a tissue engineering approach, a stem cell transplantation or any combination thereof. A medical procedure may comprise an epidural injection (such as a steroid injection), a facet joint injection, acupuncture, exercise, physical therapy, spinal surgery, an ultrasound, a facet rhizotomy, an intradiscal electrothermal annuloplasty (IDET), a radiofrequency ablation, a surgical therapy, a chiropractic manipulation, an osteopathic manipulation, a chemonucleolysis, or any combination thereof. A therapeutic can include a regenerative therapy such as a protein, a stem cell, a cord blood cell, an umbilical cord tissue, a tissue, or any combination thereof. A therapeutic can include cannabis. A therapeutic can include a biosimilar.

As used herein, “haplotype” may include a combination of genotypes on the same chromosome or different chromosome occurring in a linkage disequilibrium block. Haplotypes can serve as markers for linkage disequilibrium blocks and at the same time provide information about the arrangement of genotypes within the blocks. Typing of certain SNPs which serve as tags can, therefore, reveal all genotypes for SNPs located within a block. Thus, the use of haplotypes as tags may greatly facilitate identification of candidate genes associated with diseases and drug sensitivity.

As used herein, “linkage disequilibrium” or “LD” may include a particular combination of alleles (alternative nucleotides) or genetic markers at two or more different SNP sites may be non-randomly co-inherited (i.e., the combination of alleles at the different SNP sites occurs more or less frequently in a population than the separate frequencies of occurrence of each allele or the frequency of a random formation of haplotypes from alleles in a given population). The term “LD” may differ from “linkage,” which describes the association of two or more loci on a chromosome with limited recombination between them. LD may also be used to refer to any non-random genetic association between allele(s) at two or more different SNP sites. Therefore, when a SNP may be in LD with other SNPs, the particular allele of the first SNP often predicts which SNP sites may be present in those alleles in LD. LD may be generally, but not exclusively, due to the physical proximity of the two loci along a chromosome. Hence, genotyping one of the SNP sites may give almost the same information as genotyping the other SNP site that may be in LD. Linkage disequilibrium may be caused by fitness interactions between genes or by such non-adaptive processes as population structure, inbreeding, and stochastic effects.

Various degrees of LD can be encountered between two or more SNPs with the result being that some SNPs may be more closely associated (i.e., in stronger LD) than others. Furthermore, the physical distance over which LD extends along a chromosome differs between different regions of the genome, and therefore the degree of physical separation 20 between two or more SNP sites necessary for LD to occur can differ between different regions of the genome. In one definition, LD can be described mathematically as SNPs that have a D prime value=1 and a LOD score>2.0 or an r-squared value>0.8.

As used herein, “linkage disequilibrium block” may include a region of the genome that contains multiple SNPs located in proximity to each other and that may be transmitted as a block.

As used herein, “D prime” or D′ (also referred to as the “linkage disequilibrium measure” or “linkage disequilibrium parameter”) may include the deviation of the observed allele frequencies from the expected, and may be a statistical measure of how well a biometric system can discriminate between different individuals. The larger the D′ value, the better a biometric system may be at discriminating between individuals.

As used herein, “LOD score” may include the “logarithm of the odd” score, which may be a statistical estimate of whether two genetic loci may be physically near enough to each other (or “linked”) on a particular chromosome that they may be likely to be inherited together. A LOD score of three or more may be generally considered statistically significant evidence of linkage.

As used herein, “R-squared” or “r2” (also referred to as “correlation coefficient”) may include a statistical measure of the degree to which two markers may be related. The nearer to 1.0 the r2 value is, the more closely the markers may be related to each other. R2 cannot exceed 1.0. D prime and LOD scores generally follow the above definition for SNPs in LD. R2, however, displays a more complex pattern and can vary between about 0.0003 and 1.0 in SNPs that may be in LD. (International HapMap Consortium, Nature Oct. 27, 2005; 437:1299-1320).

The present disclosure provides SNPs associated with DDD, nucleic acid molecules containing SNPs, methods and reagents for the detection of the SNPs. The present disclosure provides methods for detecting a presence or absence of one or more SNPs in a sample obtained from a subject, diagnosing a subject as having DDD, treating a subject having or suspected of having DDD, selecting a treatment for a subject based on a result, or assessing a risk of a subject developing DDD. The present disclose also provides uses of these SNPs for the development of detection reagents, and assays or kits that utilize such reagents. The SNPs may be utilized for diagnosing, screening for, and evaluating predisposition to DDD and progression of DDD. Additionally, such SNPs may be utilized for determining individual subject treatment plans and designing of clinical trials, such as for example medical devices or pharmaceutical compositions useful in the treatment or prevention of DDD, or in the reduction in symptoms of DDD. Furthermore, such SNPs and their encoded products may be targets for the development of therapeutic agents. Furthermore, such SNPs combined with other non-genetic clinical factors such as the number of herniated discs, sciatica episodes, decreased disc height, dark nucleus pulposus and the Schneiderman or Pfirrmann grade which evaluates signal changes within the nucleus pulposus of the intervertebral discs of the lumbar spine may be useful for diagnosing, screening, evaluating predisposition to DDD, assessing risk of progression of DDD, determining individual subject treatment plans and design of clinical trials, for example, devices for possible use in the treatment of DDD.

Biological samples obtained from subjects (e.g., human subjects) may be any sample from which a genetic material (e.g., nucleic acid sample) may be derived. Samples/Genetic materials may be from biopsy, fine needle aspirate sample, spinal fluid, an extracellular fluid, a spinal or disc tissue, a buccal swab, saliva, blood, hair, nail, skin, cell, or any other type of tissue sample. In some embodiments, the genetic material (e.g., nucleic acid sample) may comprise mRNA, cDNA, genomic DNA, or PCR amplified products produced therefrom, or any combination thereof. In some embodiments, the genetic material (e.g., nucleic acid sample) may comprise PCR amplified nucleic acids produced from cDNA or mRNA. In some embodiments, the genetic material (e.g., nucleic acid sample) may comprise PCR amplified nucleic acids produced from genomic DNA. In some embodiments, the genetic material comprises a protein sample. In some embodiments, the sample may comprise a cell-free sample.

As used herein, the term “cell-free” or “cell free” may refer to the condition of the nucleic acid sequence as it appeared in the body before the sample may be obtained from the body. For example, circulating cell-free nucleic acid sequences in a sample may have originated as cell-free nucleic acid sequences circulating in the bloodstream of the human body. In contrast, nucleic acid sequences that may be extracted from a solid tissue, such as a biopsy, may be generally not considered to be “cell-free.” In some embodiments, cell-free DNA may comprise fetal DNA, maternal DNA, or a combination thereof. In some embodiments, cell-free DNA may comprise DNA fragments released into a blood plasma. In some embodiments, cell-free DNA may comprise circulating tumor DNA. In some embodiments, cell-free DNA may comprise circulating DNA indicative of a tissue origin, a disease or a condition. A cell-free nucleic acid sequence may be isolated from a blood sample. A cell-free nucleic acid sequence may be isolated from a plasma sample. A cell-free nucleic acid sequence may comprise a complementary DNA (cDNA). In some embodiments, one or more cDNAs may form a cDNA library.

The term “subject,” as used herein, may be any animal or living organism. Animals can be mammals, such as humans, non-human primates, rodents such as mice and rats, dogs, cats, pigs, sheep, rabbits, and others. A subject may be a dog. A subject may be a human. Animals can be fish, reptiles, or others. Animals can he neonatal, infant, adolescent, or adult animals. Humans can he more than about: 1, 2, 5, 10, 20, 30, 40, 50, 60, 65, 70, 75, or about 80 years of age. The subject may have or be suspected of having a condition or a disease, such as DDD. The subject may be a patient, such as a patient being treated for a condition or a disease, such as a DDD patient. The subject may be predisposed to a risk of developing a condition or a disease such as DDD. The subject may be in remission from a condition or a disease, such as a DDD patient. The subject may be healthy.

The term “sequencing” as used herein, may comprise high-throughput sequencing, next-gen sequencing, Maxam-Gilbert sequencing, massively parallel signature sequencing, Polony sequencing, 454 pyrosequencing, pH sequencing, Sanger sequencing (chain termination), Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, Enigma sequencing, sequencing-by-hybridization, sequencing-by-ligation, or any combination thereof. The sequencing output data may be subject to quality controls, including filtering for quality (e.g., confidence) of base reads. Exemplary sequencing systems include 454 pyrosequencing (454 Life Sciences), Illumina (Solexa) sequencing, SOLiD (Applied Biosystems), and Ion Torrent Systems' pH sequencing system.

Nanopores may be used to sequence, a sample, a small portion (such as one full gene or a portion of one gene), a substantial portion (such as multiple genes or multiple chromosomes), or the entire genomic sequence of an individual. Nanopore sequencing technology may be commercially available or under development from Sequenom (San Diego, Calif.), Illumina (San Diego, Calif), Oxford Nanopore Technologies LTD (Kidlington, United Kingdom), and Agilent Laboratories (Santa Clara, Calif). Nanopore sequencing methods and apparatus may be described in the art and may be provided in U.S. Pat. No. 5,795,782, herein incorporated by reference in its entirety.

Nanopore sequencing can use electrophoresis to transport a sample through a pore. A nanopore system may contain an electrolytic solution such that when a constant electric field is applied, an electric current can be observed in the system. The magnitude of the electric current density across a nanopore surface may depend on the nanopore's dimensions and the composition of the sample that is occupying the nanopore. During nanopore sequencing, when a sample approaches and or goes through the nanopore, the samples may cause characteristic changes in electric current density across nanopore surfaces, these characteristic changes in the electric current enables identification of the sample. Nanopores used herein may be solid-state nanopores, protein nanopores, or hybrid nanopores comprising protein nanopores or organic nanotubes such as carbon or graphene nanotubes, configured in a solid-state membrane, or like framework. In some embodiments, nanopore sequencing can be biological, a solid state nanopore or a hybrid biological/solid state nanopore.

In some instances, a biological nanopore can comprise transmembrane proteins that may be embedded in lipid membranes. In some embodiments, a nanopore described herein may comprise alpha hemolysin. In some embodiments, a nanopore described herein may comprise mycobacterium smegmatis porin.

Solid state nanopores do not incorporate proteins into their systems. Instead, solid state nanopore technology uses various metal or metal alloy substrates with nanometer sized pores that allow samples to pass through. Solid state nanopores may be fabricated in a variety of materials including but not limited to, silicon nitride (Si₃N₄), silicon dioxide (SiO₂), and the like. In some instances, nanopore sequencing may comprise use of tunneling current, wherein a measurement of electron tunneling through bases as sample (ssDNA) translocates through the nanopore is obtained. In some embodiments, a nanopore system can have solid state pores with single walled carbon nanotubes across the diameter of the pore. In some embodiments, nanoelectrodes may be used on a nanopore system described herein. In some embodiments, fluorescence can be used with nanopores, for example solid state nanopores and fluorescence. In such a system the fluorescence sequencing method converts each base of a sample into a characteristic representation of multiple nucleotides which bind to a fluorescent probe strand-forming dsDNA (were the sample comprises DNA). Where a two color system is used, each base can be identified by two separate fluorescences, and will therefore be converted into two specific sequences. Probes may consist of a fluorophore and quencher at the start and end of each sequence, respectively. Each fluorophore may be extinguished by the quencher at the end of the preceding sequence. When the dsDNA is translocating through a solid state nanopore, the probe strand may be stripped off, and the upstream fluorophore will fluoresce.

In some embodiments, a nanopore can comprise from about 1 nm to about 100 nm channel or an aperture may be formed through a solid substrate, usually a planar substrate, such as a membrane, through which an analyte, such as single stranded DNA, may be induced to translocate. In other embodiments, a nanopore can comprise from about 2 nm to about 50 nm channel or aperture formed through a substrate; and in still other embodiments, from about 2 nm to about 30 nm, or from about 2 nm to about 20 nm, or from about 3 nm to about 30 nm, or from about 3 nm to about 20 nm, or from about 3 nm to about 10 nm channel or aperture is formed through a substrate.

In some embodiments, nanopores used in connection with the methods and devices of the disclosure may be provided in the form of arrays, such as an array of clusters of nanopores, which may be disposed regularly on a planar surface. In some embodiments, clusters may each be in a separate resolution limited area so that optical signals from nanopores of different clusters are distinguishable by the optical detection system employed, but optical signals from nanopores within the same cluster cannot necessarily be assigned to a specific nanopore within such cluster by the optical detection system employed.

In some instances, the gene sequence may be mapped with one or more reference sequences to identify sequence variants. The base reads may be mapped against a reference sequence, which in various embodiments may be presumed to be a “normal” non-disease sequence. The DNS sequence derived from the Human Genome Project is generally used as a “premier” reference sequence. A number of mapping applications are known, and include TMAP, BWA, GSMAPPER, ELAND, MOSAIK, and MAQ. Various other alignment tools are known, and could also be implemented to map the base reads.

In some cases, based on the sequence alignments, and mapping results, sequence variants can be identified. Types of variants may include insertions, deletions, indels (a colocalized insertion and deletion), damaging mutation variants, loss of function variants, synonymous mutation variants, nonsynonymous mutation variants, nonsense mutations, recessive markers, splicing/splice-site variants, frameshift mutation, insertions, deletions, genomic rearrangements, stop-gain, stop-loss, Rare Variants (RVs), translocations, inversions, and substitutions. While the type of variants analyzed is not limited, the most numerous of the variant types will be single nucleotide substitutions, for which a wealth of data is currently available. In various embodiments, comparison of the test sequence with the reference sequence will produce at least 500 variants, at least 1000 variants, at least 3,000 variants, at least 5,000 variants, at least 10,000 variants, at least 20,000 variants, or at least 50,000 variants, but in some embodiments, will produce at least 1 million variants, at least 2 million variants, at least 3 million variants, at least 4 million variants, or at least 10 million variants. The tools provided herein enable the user to navigate the vast amounts of genetic data to identify potentially disease-causing variants.

In some cases, a wealth of data can be extracted for the identified variants, including one or more of conservation scores, genic/genomic location, zygosity, SNP ID, Polyphen, FATHMM, LRT, Mutation Accessor, and SIFT predictions, splice site predictions, amino acid properties, disease associations, annotations for known variants, variant or allele frequency data, and gene annotations. Data may be calculated and/or extracted from one or more internal or external databases. Since certain categories of annotations (e.g., amino acid properties/PolyPhen and SIFT data) are dependent on a nature of the region of the genome in which they are contained (e.g., whether a variant is contained within a region translated to give rise to an amino acid sequence in a resultant protein), these annotations can be carried out for each known transcript. Exemplary external databases include OMIM (Online Mendelian Inheritance in Man), HGMD (The Human Gene Mutation Database), PubMed, PolyPhen, SIFT, SpliceSite, reference genome databases, the University of California Santa Cruz (UCSC) genome database, CLINVAR database, the BioBase biological databases, the dbSNP Short Genetic Variations database, the Rat Genome Database (RGD), and/or the like. Various other databases may be employed for extracting data on identified variants. Variant information may be further stored in a central data repository, and the data extracted for future sequence analyses.

The term “homology” can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which may correspond with a particular nucleic acid sequence described herein), such particular polypeptide sequence can be determined using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity is calculated over the full length of the reference sequence and that gaps in homology of up to 5% of the total reference sequence are allowed.

In some embodiments, the identity between a reference sequence (query sequence, i.e., a sequence of the present disclosure) and a subject sequence, also referred to as a global sequence alignment, may be determined using the FASTDB computer program based on the algorithm of Brutlag et al. (Comp. App. Biosci. 6:237-245 (1990)). In some embodiments, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some embodiments, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. A 90 residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

### Analysis of Rare Mutations in Sequenced Genes

In some embodiments, the present disclosure provides an analysis to evaluate a coding region of a gene as a component of a genetic diagnostic or predictive test for degenerative disc disease. In some embodiments, the analysis can comprise one or more of the approaches.

In some embodiments, the analysis can comprise performing DNA variant search on a next generation sequencing output file using a standard software designed for this purpose, for example Life Technologies TMAP algorithm with their default parameter settings, and Life Technologies Torrent Variant Caller software. ANNOVAR can be used to classify coding variants as synonymous, missense, frameshift, splicing, stop-gain, or stop-loss. Variants can be considered “loss-of-function” if the variant causes a stop-loss, stop-gain, splicing, or frame-shift insertion or deletion).

In some embodiments, the analysis can comprise evaluating prediction of an effect of each variant on protein function in silico using a variety of different software algorithms: Polyphen 2, Sift, Mutation Accessor, Mutation Taster, FATHMM, LRT, MetaLR, or any combination thereof. Missense variants can be deemed “damaging” if they may be predicted to be damaging by at least one of the seven algorithms tested.

In some embodiments, the analysis can comprise searching population databases (e.g., gnomAD) and proprietary degenerative disc disease allele frequency databases for the prevalence of any loss of function or damaging mutations identified by these analyses. The log of the odds ratio can be used to weight the marker when the variant has been previously observed in the reference databases. When a damaging variant or loss of function variant may not have been reported in the reference databases, a default odds ratio of 10 can be used to weight the finding.

In some embodiments, the analysis can comprise incorporating findings into the Risk Score as with the other low-frequency alleles. Risk Score=Summation [log(OR)×Count], where count equals the number of low frequency alleles detected at each degenerative disc disease associated locus. Risk scores can be converted to probability using a nomogram based on confirmed diagnoses.

In some embodiments, the methods can provide a high sensitivity of detecting gene mutations and diagnosing degenerative disc disease that may be greater than about: 60%, 65%, 70%, 75%, 80%, 85%, 90%, 91%, 92%, 93%, 94%, 95%, 95.5%, 96%, 96.5%, 97%, 97.5%, 98%, 98.5%, 99%, 99.5% or more. In some embodiments, the methods can provide a high specificity of detecting and classifying gene mutations and degenerative disc disease, for example, greater than about: 80%, 85%, 90%, 91%, 92%, 93%, 94%, 95%, 95.5%, 96%, 96.5%, 97%, 97.5%, 98%, 98.5%, 99%, 99.5% or more. In some embodiments, a nominal specificity for the method can be greater than or equal to about 70%. In some embodiments, a nominal Negative Predictive Value (NPV) for the method can be greater than or equal to about 95%. In some embodiments, a NPV for the method can be about: 95%, 95.5%, 96%, 96.5%, 97%, 97.5%, 98%, 98.5%, 99%, 99.5% or more. In some embodiments, a nominal Positive Predictive Value (PPV) for the method can be greater than or equal to 95%. In some embodiments, a PPV for the method can be about: 95%, 95.5%, 96%, 96.5%, 97%, 97.5%, 98%, 98.5%, 99%, 99.5% or more. In some embodiments, the accuracy of the methods in diagnosing degenerative disc disease can be greater than about: 70%, 75%, 80%, 85%, 90%, 91%, 92%, 93%, 94%, 95%, 95.5%, 96%, 96.5%, 97%, 97.5%, 98%, 98.5%, 99%, 99.5% or more.

### Methods of Detection of Variants

The methods and kits as described herein may include detecting a presence of a variant allele. The variant allele detected may be a reference allele, an alternative allele, a non-reference allele, a major allele, a minor allele, or any combination thereof. In some cases, one or more minor alleles are detected. In some cases, a major allele is detected. In some cases, one or more minor alleles and a major allele are detected.

A major allele may be a variant allele that occurs with greater than 50% frequency in a population of subjects. A variant allele may or may not be a major allele depending on the population of subjects. A major allele may be present in about: 50.5%, 55%, 60%, 65%, 70%, 75%, 80%, 85%, 90%, 95%, 96%, 97%, 98%, 99%, 99.5% of a population. A major allele may be present in from about 50.5% to about 99.9% of a population. A major allele may be present in from about 50.5% to about 80% of a population. A major allele may be present in from about 50.5% to about 70% of a population. A major allele may be present in from about 50.5% to about 60% of a population. A major allele may be present in from about 55% to about 99.9% of a population. A major allele may be present in from about 60% to about 99.9% of a population. A major allele may be present in from about 70% to about 99.9% of a population. A major allele may be present in from about 80% to about 99.9% of a population.

A minor allele may be a variant allele that occurs with less than 50% frequency in a population of subjects. A variant allele may or may not be a minor allele depending on the population of subjects. A minor allele may be present in about: 49.5%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.01% of a population. A minor allele may be present in from about 49.5% to about 0.1% of a population. A minor allele may be present in from about 40% to about 0.1% of a population. A minor allele may be present in from about 30% to about 0.1% of a population. A minor allele may be present in from about 20% to about 0.1% of a population. A minor allele may be present in from about 10% to about 0.1% of a population. A minor allele may be present in from about 5% to about 0.1% of a population. A minor allele may be present in from about 1% to about 0.01% of a population. A minor allele may be present in from about 0.5% to about 0.01% of a population. A minor allele may be present in from about 0.3% to about 0.01% of a population. A minor allele may be present in from about 0.2% to about 0.01% of a population.

A reference allele may be selected or assigned. A reference allele may be a major allele. A reference allele may not be a major allele. A reference allele may be an ancestral allele. A reference allele may be a major allele from a general population of subjects. A reference allele may be compared to an alternative allele or non-reference allele. An alternative or non-reference allele may be a minor allele. An alternative or non-reference allele may not be a minor allele. In some cases, there may be more than one alternative or non-reference allele, such as 2, 3, 4, or more alternative or non-reference alleles. More than one alternative or non-reference allele may represent a plurality of minor alleles.

A reference allele, an alternative allele, a non-reference allele, a major allele, a minor allele, or any combination thereof may be defined by a population from which a variant allele is detected. A population of subjects may be representative of a general population. A population of subjects may be representative of individuals having been diagnosed with DDD or suffering from symptoms of DDD. A major and minor allele may vary depending on the population selected. A population may be defined by one or more of: a size, a distribution of: age, health status, gender, ethnicity, geographical location, or any combination thereof.

A population size may be about: 5, 10, 20, 30, 40, 50, 60, 70, 80, 90, 100, 125, 150, 175, 200, 250, 500, 1000, 2500, 5000, 10,000, 25,000, 50,000, 75,000, 100,000, 250,000, 500,000, 750,000, or 100,000,000 subjects. A population may comprise females, males, or both. A population may comprise healthy individuals or individuals having been diagnosed with a disease or condition or a combination thereof. A population may include individuals of a same ethnicity or a different ethnicity. A population may include individuals of a same geographical location or a different geographical location. A population may include infants, children, adolescents, young adults, middle aged adults, elderly subjects, or any combination thereof.

In some cases, a population may be representative of a general population or at least a portion of a general population. A population may be a global population. The reference allele may be the major allele, occurring in greater than 50% of the general population. The non-reference or alternative allele may be the minor allele, occurring in less than 50% of the general population, such as a rare minor allele occurring in less than about 5%, 4%, 3%, 2%, or 1% of a general population. Individuals identified as having the minor allele may be individuals that have an increased risk of developing DDD or individuals that have DDD.

In some cases, a population may be representative of a selected population of individuals, such as individuals suffering from DDD or having been previously diagnosed with DDD. The reference allele may be major allele, occurring in greater than 50% of the selected population. Individuals having the major allele may be indicative of a presence of DDD or a risk of developing DDD. The non-reference allele or alternative allele, occurring in less than 50% of the selected population may be indicative of non-diagnostic variant or indicative of a subtype of DDD that may occur in a subset of individuals.

In some aspects, the present disclosure can provide methods to detect variants, e.g, detecting a genetic variant in a panel comprising two or more genetic variants defining a minor allele, an alternative allele, or a non-reference allele (e.g., in FIG. 1). In some embodiments, the detecting can comprise, DNA sequencing, hybridization with a complementary probe, an oligonucleotide ligation assay, a PCR-based assay, or any combination thereof. In some embodiments, the panel can comprise at least about: 2, 3, 4, 5, 6, 7, 8, 9, 10, 15, 20, 25, 30, 35, 40, 45, 50, 60, 70, 75, 80, 90, 100, 150, 200, 250, 300, 350, 400, 450, 500, or more genetic variants defining minor alleles, alternative alleles, or non-reference alleles (e.g., in FIG. 1). In some embodiments, the genetic variant to detect or detected can have an odds ratio (OR) of at least about: 0.1, 1, 1.5, 2, 5, 10, 20, 50, 100, 127, 130, 140, 150, 200, 300, 400, 500, 600, 700, 800, 900, 1000, 1500, or more.

In some embodiments, variants may include single nucleotide polymorphisms (SNPs), insertion deletion polymorphisms (indels), damaging mutation variants, loss of function variants, synonymous mutation variants, nonsynonymous mutation variants, nonsense mutations, recessive markers, splicing/splice-site variants, frameshift mutation, insertions, deletions, genomic rearrangements, stop-gain, stop-loss, Rare Variants (RVs), translocations, inversions, an substitutions.

Variants for example SNPs may be usually preceded and followed by highly conserved sequences that vary in less than 1/100 or 1/1000 members of the population. An individual may be homozygous or heterozygous for an allele at each SNP position. A SNP may, in some embodiments, be referred to as a “cSNP” to denote that the nucleotide sequence containing the SNP may be an amino acid “coding” sequence. A SNP may arise from a substitution of one nucleotide for another at the polymorphic site. Substitutions can be transitions or transversions. A transition may be the replacement of one purine nucleotide by another purine nucleotide, or one pyrimidine by another pyrimidine. A transversion may be the replacement of a purine by a pyrimidine, or vice versa.

A synonymous codon change or silent mutation may be one that does not result in a change of amino acid due to the degeneracy of the genetic code. A substitution that changes a codon coding for one amino acid to a codon coding for a different amino acid (i.e., a non-synonymous codon change) may be referred to as a missense mutation. A nonsense mutation results in a type of non-synonymous codon change in which a stop codon may be formed, thereby leading to premature termination of a polypeptide chain and a truncated protein. A read-through mutation may be another type of non-synonymous codon change that causes the destruction of a stop codon, thereby resulting in an extended polypeptide product. An indel that occur in a coding DNA segment can give rise to a frameshift mutation.

Causative variants may be those that produce alterations in gene expression or in the structure and/or function of a gene product, and therefore may be predictive of a possible clinical phenotype. One such class includes SNPs falling within regions of genes encoding a polypeptide product, i.e. cSNPs. These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Such variant products can result in a pathological condition, e.g., degenerative disc disease.

An association study of a variant and a specific disorder involves determining the presence or frequency of the variant allele in biological samples from individuals with the disorder of interest, such as degenerative disc disease, and comparing the information to that of controls (i.e., individuals who may not have the disorder; controls may also be referred to as “healthy” or “normal” individuals) who may be of similar age and race. The appropriate selection of patients and controls may be important to the success of variant association studies. Therefore, a pool of individuals with well-characterized phenotypes may be extremely desirable.

A variant may be screened in tissue samples or any biological sample obtained from an affected subject, and compared to control samples or a reference, and selected for its increased (or decreased) occurrence in a specific pathological condition, such as pathologies related to degenerative disc disease. Once a statistically significant association may be established between one or more variant(s) and a pathological condition (or other phenotype) of interest, then the region around the variant can be thoroughly screened to identify the causative genetic locus/sequence(s) (e.g., causative variant/mutation, gene, regulatory region, etc.) that influences the pathological condition or phenotype. Association studies may be conducted within the general population. For diagnostic and prognostic purposes, if a particular variant site may be found to be useful for diagnosing a disease, such as degenerative disc disease, other variant sites which may be in LD with this variant site may also be expected to be useful for diagnosing the condition. Linkage disequilibrium may be described in the human genome as blocks of variants along a chromosome segment that may not segregate independently (i.e., that may be non-randomly co-inherited). The starting (5′ end) and ending (3′ end) of these blocks can vary depending on the criteria used for linkage disequilibrium in a given database, such as the value of D′ or r² used to determine linkage disequilibrium.

In some embodiments, variants can be identified in a study using a whole-genome case-control approach to identify single nucleotide polymorphisms that were closely associated with the development of degenerative disc disease (DDD), as well as variants found to be in linkage disequilibrium with (i.e., within the same linkage disequilibrium block as) the DDD-associated variants, which can provide haplotypes (i.e., groups of variants that may be co-inherited) to be readily inferred. Thus, the present disclosure provides individual variants associated with DDD, as well as combinations of variants and haplotypes in genetic regions associated with DDD, methods of detecting these polymorphisms in a test sample, methods of determining the risk of an individual of having or developing DDD and for clinical sub-classification of DDD.

In some embodiments, the present disclosure provides variants associated with DDD, as well as variants that were previously known in the art, but were not previously known to be associated with DDD. Accordingly, the present disclosure provides novel compositions and methods based on the variants, and also provides novel methods of using the known but previously unassociated variants in methods relating to DDD (e.g., for diagnosing DDD etc.).

In some embodiments, particular variant alleles can be associated with either an increased risk of having or developing degenerative disc disease, or a decreased risk of having or developing degenerative disc disease or determining a stage of DDD. Variant alleles that may be associated with a decreased risk may be referred to as “protective” alleles, and variant alleles that may be associated with an increased risk may be referred to as “susceptibility” alleles, “risk factors”, or “high-risk” alleles. Thus, whereas certain variants can be assayed to determine whether an individual possesses a variant allele that may be indicative of an increased risk of having or developing degenerative disc disease (i.e., a susceptibility allele), other variants can be assayed to determine whether an individual possesses a variant allele that may be indicative of a decreased risk of having or developing degenerative disc disease (i.e., a protective allele). Similarly, particular variant alleles can be associated with either an increased or decreased likelihood of responding to a particular treatment. The term “altered” may be used herein to encompass either of these two possibilities (e.g., an increased or a decreased risk/likelihood).

In some embodiments, nucleic acid molecules may be double-stranded molecules and that reference to a particular site on one strand refers, as well, to the corresponding site on a complementary strand. In defining a variant position, variant allele, or nucleotide sequence, reference to an adenine, a thymine (uridine), a cytosine, or a guanine at a particular site on one strand of a nucleic acid molecule also defines the complementary thymine (uridine), adenine, guanine, or cytosine (respectively) at the corresponding site on a complementary strand of the nucleic acid molecule. Thus, reference may be made to either strand in order to refer to a particular variant position, variant allele, or nucleotide sequence. Probes and primers may be designed to hybridize to either strand and variant genotyping methods may generally target either strand. Throughout the specification, in identifying a variant position, reference may be generally made to the forward or “sense” strand, solely for the purpose of convenience. Since endogenous nucleic acid sequences exist in the form of a double helix (a duplex comprising two complementary nucleic acid strands), it may be understood that the variants may have counterpart nucleic acid sequences and variants associated with the complementary “reverse” or “antisense” nucleic acid strand. Such complementary nucleic acid sequences, and the complementary variants present in those sequences, may be included within the scope of the present disclosure.

Disclosed herein may be methods for detecting genetic variants in a nucleic acid sample. The method can comprise sequencing a nucleic acid sample obtained from a subject having degenerative disc disease or suspected of having degenerative disc disease using a high throughput method. The high throughput method can comprise nanopore sequencing. The method can comprise detecting one or more genetic variants in a nucleic acid sample, wherein the one or more genetic variants may be listed in FIG. 1. The nucleic acid sample can comprise RNA. The RNA can comprise mRNA. The RNA can comprise cell-free RNA. The RNA can comprise miRNA, The nucleic acid sample can comprise DNA. The DNA can comprise cDNA, genomic DNA, sheared DNA, cell free DNA, fragmented DNA, or PCR amplified products produced therefrom, or any combination thereof. The one or more genetic variants can comprise a genetic variant defining a minor allele, an alternative allele, or a non-reference allele. The one or more genetic variants can comprise at least about: 5, 10, 15, 20, 25, 50, 75, 100, 150, 200, 250, 500, or more genetic variants defining minor alleles, alternative alleles, or non-reference alleles. The detection of the one or more genetic variants can have an odds ratio (OR) for degenerative disc disease of at least about: 1.5, 2, 5, 10, 20, 50, 100, or more. The one or more genetic variants can comprise a synonymous mutation, a non-synonymous mutation, a stop-gain mutation, a nonsense mutation, an insertion, a deletion, a splice-site variant, a frameshift mutation, or any combination thereof. The one or more genetic variants can comprise a protein damaging mutation. The one or more genetic variants can be identified based on a predictive computer algorithm. The one or more genetic variants can be identified based on reference to a database. The method can further comprise identifying a subject as having degenerative disc disease or being at risk of developing degenerative disc disease. The method can comprise identifying a subject as having degenerative disc disease or being at risk of developing degenerative disc disease with a specificity of at least about: 80%, 85%, 90%, 95%, 96%, 97%, 98%, or 99%. The method can comprise identifying a subject as having degenerative disc disease or being at risk of developing degenerative disc disease with a sensitivity of at least about: 80%, 85%, 90%, 95%, 96%, 97%, 98%, or 99%. The method can comprise identifying a subject as having degenerative disc disease or being at risk of developing degenerative disc disease with an accuracy of at least about: 80%, 85%, 90%, 95%, 96%, 97%, 98%, or 99%. The method can comprise identifying a subject as having degenerative disc disease. The subject can be asymptomatic for degenerative disc disease. In some embodiments, the subject can have degenerative disc disease and be asymptomatic. The subject can be symptomatic for degenerative disc disease. The subject can be identified as being at risk of developing degenerative disc disease. The method can further comprise administering a therapeutic to a subject. The therapeutic can comprise a pain medication. The pain medication can comprise a nonsteroidal anti-inflammatory drug (NSAID), ibuprofen, naproxen, an opioid, a cannabis-based therapeutic, or any combination thereof. In some embodiments, the one or more genetic variants may be listed in FIG. 1. A subject described herein can be a mammal. The mammal can be a human. Nanopore sequencing can be performed with a biological nanopore, a solid state nanopore, or a hybrid nanopore. Methods can detect about: 1, 5, 10, 15, 20, 30, 50, 60, 100, 80, 90, 100, 200 or more variants. Genetic variants detected herein can indicate degenerative disc disease or a risk of developing degenerative disc disease. In some embodiments, one or more genetic variant listed in FIG. 1 may be the only genetic variants detected.

### Genotyping Methods

In some embodiments, the process of determining which specific nucleotide (i.e., allele) is present at each of one or more variant positions, such as a variant position in a nucleic acid molecule characterized by a variant, may be referred to as variant genotyping. The present disclosure provides methods of variant genotyping, such as for use in screening for degenerative disc disease or related pathologies, or determining predisposition thereto, or determining responsiveness to a form of treatment, or in genome mapping or variant association analysis, etc.

Nucleic acid samples can be genotyped to determine which allele(s) is/are present at any given genetic region (e.g., variant position) of interest by methods well known in the art. The neighboring sequence can be used to design variant detection reagents such as oligonucleotide probes, which may be implemented in a kit format. Variant genotyping methods may include TaqMan assays, molecular beacon assays, nucleic acid arrays, allele-specific primer extension, allele-specific PCR, arrayed primer extension, homogeneous primer extension assays, primer extension with detection by mass spectrometry, mass spectrometry with or with monoisotopic dNTPs (pyrosequencing, multiplex primer extension sorted on genetic arrays, ligation with rolling circle amplification, homogeneous ligation, OLA, multiplex ligation reaction sorted on genetic arrays, restriction-fragment length polymorphism, single base extension-tag assays, and the Invader assay. Such methods may be used in combination with detection mechanisms such as, luminescence or chemiluminescence detection, fluorescence detection, time-resolved fluorescence detection, fluorescence resonance energy transfer, fluorescence polarization, mass spectrometry, electrospray mass spectrometry, and electrical detection.

Various methods for detecting polymorphisms can include methods in which protection from cleavage agents may be used to detect mismatched bases in RNA/RNA or RNA/DNA duplexes, comparison of the electrophoretic mobility of variant and wild type nucleic acid molecules, and assaying the movement of polymorphic or wild-type fragments in polyacrylamide gels containing a gradient of denaturant using denaturing gradient gel electrophoresis (DGGE). Sequence variations at specific locations can also be assessed by nuclease protection assays such as RNase and SI protection or chemical cleavage methods.

In some embodiments, a variant genotyping can be performed using the TaqMan assay, which may be known as the 5′ nuclease assay. The TaqMan assay detects the accumulation of a specific amplified product during PCR. The TaqMan assay utilizes an oligonucleotide probe labeled with a fluorescent reporter dye and a quencher dye. The reporter dye may be excited by irradiation at an appropriate wavelength, and may transfer energy to the quencher dye in the same probe via a process called fluorescence resonance energy transfer (FRET). In some embodiments, when attached to the probe, the excited reporter dye does not emit a signal. In some embodiments, the proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye may be attached to an internal nucleotide, or vice versa. In some embodiments, both the reporter and the quencher may be attached to internal nucleotides at a distance from each other such that fluorescence of the reporter may be reduced. During PCR, the 5′ nuclease activity of DNA polymerase may cleave the probe, thereby separating the reporter dye and the quencher dye and resulting in increased fluorescence of the reporter. Accumulation of PCR product may be detected directly by monitoring the increase in fluorescence of the reporter dye. The DNA polymerase may cleave the probe between the reporter dye and the quencher dye only if the probe hybridizes to the target variant-containing template which may be amplified during PCR, and the probe may be designed to hybridize to the target variant site only if a particular variant allele may be present. TaqMan primer and probe sequences can readily be determined using the variant and associated nucleic acid sequence information. A number of computer programs, such as Primer Express (Applied Biosystems, Foster City, Calif.), can be used to rapidly obtain optimal primer/probe sets. It may be apparent to one of skill in the art that such primers and probes for detecting the variants in diagnostic assays for degenerative disc disease and related pathologies, and can be readily incorporated into a kit format. The present disclosure also includes modifications of the Taqman assay well known in the art such as the use of Molecular Beacon probes and other variant formats.

In some embodiments, a method for genotyping the variants can be the use of two oligonucleotide probes in an OLA. In this method, one probe can hybridize to a segment of a target nucleic acid with its 3′ most end aligned with the variant site. A second probe can hybridize to an adjacent segment of the target nucleic acid molecule directly 3′ to the first probe. The two juxtaposed probes can hybridize to the target nucleic acid molecule, and may be ligated in the presence of a linking agent such as a ligase if there is perfect complementarity between the 3′ most nucleotide of the first probe with the variant site. If there is a mismatch, ligation may not occur. After the reaction, the ligated probes may be separated from the target nucleic acid molecule, and detected as indicators of the presence of a variant.

In some embodiments, a method for variant genotyping is based on mass spectrometry. Mass spectrometry takes advantage of the unique mass of each of the four nucleotides of DNA. Variants can be unambiguously genotyped by mass spectrometry by measuring the differences in the mass of nucleic acids having alternative variant alleles. MALDI-TOF (Matrix Assisted Laser Desorption Ionization-Time of Flight) mass spectrometry technology may be exemplary for extremely precise determinations of molecular mass, such as variants. Numerous approaches to variant analysis have been developed based on mass spectrometry. Exemplary mass spectrometry-based methods of variant genotyping include primer extension assays, which can also be utilized in combination with other approaches, such as traditional gel-based formats and microarrays.

In some embodiments, a method for genotyping the variants can include the use of electrospray mass spectrometry for direct analysis of an amplified nucleic acid. In this method, in one aspect, an amplified nucleic acid product may be isotopically enriched in an isotope of oxygen (O), carbon (C), nitrogen (N) or any combination of those elements. In an exemplary embodiment the amplified nucleic acid may be isotopically enriched to a level of greater than about 99.9% in the elements of O¹⁶, C¹² and N¹⁴.The amplified isotopically enriched product can then be analyzed by electrospray mass spectrometry to determine the nucleic acid composition and the corresponding variant genotyping. Isotopically enriched amplified products can result in a corresponding increase in sensitivity and accuracy in the mass spectrum. In another aspect of this method, an amplified nucleic acid that may not be isotopically enriched can also have composition and variant genotype determined by electrospray mass spectrometry.

In some embodiments, variants can be scored by direct DNA sequencing or the use of next generation sequencing. The nucleic acid sequences may enable one of ordinary skill in the art to readily design sequencing primers for such automated sequencing procedures. Commercial instrumentation, such as the Applied Biosystems 377, 3100, 3700, 3730, and 3730.times.1 DNA Analyzers (Foster City, Calif.), may be used in the art for automated sequencing.

Variant genotyping can include the steps of collecting a biological sample from a human subject (e.g., sample of tissues, cells, fluids, secretions, etc.), isolating nucleic acids (e.g., genomic DNA, mRNA or both) from the cells of the sample or from a cell free sample, contacting the nucleic acids with one or more primers which specifically hybridize to a region of the isolated nucleic acid containing a target variant under conditions such that hybridization and amplification of the target nucleic acid region occurs, and determining the nucleotide present at the variant position of interest, or, in some assays, detecting the presence or absence of an amplification product (assays can be designed so that hybridization and/or amplification may only occur if a particular variant allele may be present or absent). In some assays, the size of the amplification product may be detected and compared to the length of a control sample or a reference; deletions and insertions can be detected by a change in size of the amplified product compared to a normal genotype.

In some embodiments, a variant genotyping can be used in applications that may include variant-degenerative disc disease association analysis, degenerative disc disease predisposition screening, degenerative disc disease diagnosis, degenerative disc disease prognosis, degenerative disc disease progression monitoring, determining therapeutic strategies based on an individual's genotype, and stratifying a patient population for clinical trials for a treatment such as minimally invasive device for the treatment of degenerative disc disease.

### Single Nucleotide Polymorphisms (SNPs)

As used herein, the term SNP may refer to single nucleotide polymorphisms in DNA. SNPs may be usually preceded and followed by highly conserved sequences that vary in less than 1/100 or 1/1000 members of the population. An individual may be homozygous or heterozygous for an allele at each SNP position. A SNP may, in some instances, be referred to as a “cSNP” to denote that the nucleotide sequence containing the SNP may be an amino acid “coding” sequence.

A SNP may arise from a substitution of one nucleotide for another at the polymorphic site. Substitutions can be transitions or transversions. A transition may be the replacement of one purine nucleotide by another purine nucleotide, or one pyrimidine by another pyrimidine. A transversion may be the replacement of a purine by a pyrimidine, or vice versa. A SNP may also be a single base insertion or deletion variant referred to as an “indel.”

A synonymous codon change, or silent mutation SNP (terms such as “SNP”, “polymorphism”, “mutation”, “mutant”, “variation”, and “variant” may be used herein interchangeably), may be one that does not result in a change of amino acid due to the degeneracy of the genetic code. A substitution that changes a codon coding for one amino acid to a codon coding for a different amino acid (i.e., a non-synonymous codon change) may be referred to as a mis-sense mutation. A nonsense mutation results in a type of non-synonymous codon change in which a stop codon may be formed, thereby leading to premature termination of a polypeptide chain and a truncated protein. A read-through mutation may be another type of non-synonymous codon change that causes the destruction of a stop codon, thereby resulting in an extended polypeptide product. While SNPs can be bi-, tri-, or tetra-allelic, the vast majority of the SNPs may be bi-allelic and may be referred to as “bi-allelic markers” or “di-allelic markers”.

As used herein, references to SNPs and SNP genotypes include individual SNPs and/or haplotypes, which may be groups of SNPs that are generally inherited together. Haplotypes can have stronger correlations with diseases or other phenotypic effects compared with individual SNPs, and therefore may provide increased diagnostic accuracy in some cases.

Causative SNPs may be those SNPs that produce alterations in gene expression or in the expression, structure, and/or function of a gene product, and therefore may be most predictive of a possible clinical phenotype. One such class includes SNPs falling within regions of genes encoding a polypeptide product, i.e. cSNPs. These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Such variant products can result in a pathological condition, e.g., genetic DDD.

Causative SNPs may not necessarily have to occur in coding regions; causative SNPs can occur in any genetic region that can ultimately affect the expression, structure, and/or activity of the protein encoded by a nucleic acid. Such genetic regions may include those involved in transcription, such as SNPs in transcription factor binding domains, SNPs in promoter regions, in areas involved in transcript processing, such as SNPs at intron-exon boundaries that may cause defective splicing, or SNPs in mRNA processing signal sequences such as polyadenylation signal regions. Some SNPs that may not be causative SNPs nevertheless may be in close association with, and therefore segregate with, a disease-causing sequence. In this situation, the presence of a SNP correlates with the presence of, or predisposition to, or an increased risk in developing the DDD. These SNPs, although not causative, may be nonetheless also useful for diagnostics, DDD predisposition screening, DDD progression risk and other uses.

An association study of a SNP and a specific disorder involves determining the presence or frequency of the SNP allele in biological samples from individuals with the disorder of interest, such as DDD and comparing the information to that of controls (i.e., individuals who may not have the disorder; controls may be referred to as “healthy” or “normal” subjects) who may be of similar age and race. The appropriate selection of subjects and controls may be important to the success of SNP association studies. Therefore, a pool of individuals with well-characterized phenotypes may be extremely desirable.

A SNP may be screened in tissue samples or any biological sample obtained from an affected subject, and compared to control samples/references, and selected for its increased (or decreased) occurrence in a specific pathological condition, such as pathologies related to DDD. Once a statistically significant association may be established between one or more SNP(s) and a pathological condition (or other phenotype) of interest, then the region around the SNP can be thoroughly screened to identify the causative genetic locus/sequence(s) (e.g., causative SNP/mutation, gene, regulatory region, etc.) that influences the pathological condition or phenotype. Association studies may be conducted within the general population and may not be limited to studies performed on related individuals in affected families (linkage studies).

For diagnostic and prognostic purposes, if a particular SNP site may be found to be useful for diagnosing a disease, such as DDD, other SNP sites which may be in LD with this SNP site may also be expected to be useful for diagnosing the condition. Linkage disequilibrium may be described in the human genome as blocks of SNPs along a chromosome segment that may not segregate independently (i.e., that may be non-randomly co-inherited). The starting (5′ end) and ending (3′ end) of these blocks can vary depending on the criteria used for linkage disequilibrium in a given database, such as the value of D′ or r2 used to determine linkage disequilibrium.

By way of example, FIG. 1 lists 276 SNPs associated with DDD. Furthermore, the SNPs that may be in the same linkage disequilibrium block as one of the 276 SNPs in FIG. 1 may also be useful, either individually, in combination with one of the 276 SNPs in FIG. 1 or in a haplotype involving one of the 276 SNPs in FIG. 1. Linkage disequilibrium blocks can be identified in a number of ways such as the SNPbrowser software (v3.5, Applera, Inc., Foster City, Calif.). SNPbrowser may be a linkage disequilibrium-guided tool for selection of SNPs. The linkage disequilibrium blocks in SNPbrowser may be based on the International HapMap Consortium data and D′ values of linkage disequilibrium.

In accordance with the present disclosure, SNPs have been identified in a study using a whole-genome case-control approach to identify single nucleotide polymorphisms that were closely associated with the development of DDD and specifically progression or non-progression risk of DDD. FIG. 1 identifies 276 SNPs associated with DDD. In addition, SNPs found to be in linkage disequilibrium with (i.e., within the same linkage disequilibrium block as) the DDD-associated SNPs of FIG. 1 can provide haplotypes (i.e., groups of SNPs that may be co-inherited) to be readily inferred. The present disclosure encompasses SNP haplotypes (combinations of SNPs), as well as individual SNPs.

Thus, the present disclosure provides individual SNPs associated with DDD, as well as combinations of SNPs and haplotypes in genetic regions associated with DDD, methods of detecting these polymorphisms in a test sample, methods of determining the risk of an individual of having or developing DDD and developing progressive DDD.

Particular SNP alleles can be associated with either an increased risk of having or developing DDD, or a decreased risk of having or developing DDD, or an increased risk of developing progressive DDD, or a decreased risk of developing progressive DDD, or determining a stage of DDD. SNP alleles that may be associated with a decreased risk may be referred to as “protective” alleles, and SNP alleles that may be associated with an increased risk may be referred to as “susceptibility” alleles, “risk factors”, or “high-risk” alleles. Thus, whereas certain SNPs can be assayed to determine whether an individual possesses a SNP allele that may be indicative of an increased risk of having or developing DDD or progressive DDD (i.e., a susceptibility allele), other SNPs can be assayed to determine whether an individual possesses a SNP allele that may be indicative of a decreased risk of having or developing DDD or progressive DDD (i.e., a protective allele). Similarly, particular SNP alleles can be associated with either an increased or decreased likelihood of responding to a particular treatment. The term “altered” may be used herein to encompass either of these two possibilities (e.g., an increased or a decreased risk/likelihood).

Those skilled in the art may readily recognize that nucleic acid molecules may be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the complementary thymine (uridine), adenine, guanine, or cytosine (respectively) at the corresponding site on a complementary strand of the nucleic acid molecule. Thus, reference may be made to either strand in order to refer to a particular SNP position, SNP allele, or nucleotide sequence. Probes and primers may be designed to hybridize to either strand and SNP genotyping methods may generally target either strand. One or more primers may be designed to comprise at least: 80%, 85%, 90%, 95%, 96%, 97% or more sequence complementarity to at least a portion of any one of SNP 1 through SNP 276 of FIG. 1, or any combination thereof. Throughout the specification, in identifying a SNP position, reference may be generally made to the forward or “sense” strand, solely for the purpose of convenience. Since endogenous nucleic acid sequences exist in the form of a double helix (a duplex comprising two complementary nucleic acid strands), it may be understood that the SNPs may have counterpart nucleic acid sequences and SNPs associated with the complementary “reverse” or “antisense” nucleic acid strand. Such complementary nucleic acid sequences, and the complementary SNPs present in those sequences, may be included within the scope of the present disclosure.

The present disclosure provides methods for utilizing the SNPs disclosed in FIG. 1 for determining whether a human subject has DDD, may be at risk of developing DDD or may be at risk of DDD progression. In some embodiments, the methods of the disclosure comprise the step of detecting in the genetic material of said subject the presence or absence of one or more protective or high-risk polymorphism selected from the group consisting of the polymorphisms of FIG. 1 or a polymorphism that may be in linkage disequilibrium with a polymorphism of FIG. 1, wherein the polymorphism may be correlated with DDD, altered risk of developing DDD or altered risk of DDD progression. In some embodiments, the polymorphism is selected from the polymorphisms of FIG. 1.

In other embodiments, the methods further comprise the step of evaluating the risk associated with one or more non-genetic clinical factors selected from the group consisting of the number of herniated discs, sciatica episodes, decreased disc height, dark nucleus pulposus and the Schneiderman or Pfirrmann grade which evaluates signal changes within the nucleus pulposus of the intervertebral discs of the lumbar spine and other factors associated with DDD.

In other embodiments, the method of detecting in a nucleic acid molecule a polymorphism that may be correlated with DDD, altered risk of developing DDD or altered risk of DDD progression, comprises contacting a test sample with a polynucleotide sequence that specifically hybridizes under stringent hybridization conditions to a polynucleotide sequence having one or more protective or high-risk polymorphism selected from the group consisting of the polymorphisms of FIG. 1 or a polymorphism that may be in linkage disequilibrium with a polymorphism of FIG. 1 or a complement thereof, wherein the polymorphism may be correlated with DDD, altered risk of developing DDD or altered risk of DDD progression, and detecting the formation of a hybridized duplex.

With respect to the above methods, the polymorphism may be correlated with an increased risk of DDD progression in a human subject having a degenerative disc or DDD.

The above methods may further comprise the step of correlating the polymorphism with an appropriate medical treatment, including the use of medical devices or pharmaceuticals, in a human subject having DDD or who has been determined to be at risk for DDD or DDD progression.

The above methods may further comprise the step of selecting human subjects for clinical trials involving either medical devices or pharmaceuticals for use in the treatment of DDD.

In the above methods, the polymorphism may be correlated with presymptomatic risk of developing DDD in a human subject. The human subject may be an adult or may be a human fetus.

In the above methods, the step of assessing DDD risk may be by determining whether each of a set of independent variables has a unique predictive relationship to a dichotomous dependent variable. The step of assessing DDD risk may comprise an algorithm comprising a logistic regression analysis.

### Amplified Nucleic Acid Molecules

The present disclosure further provides amplified polynucleotides containing the nucleotide sequence of a polymorphism selected from the polymorphisms of FIG. 1 or a polymorphism that may be in linkage disequilibrium with a polymorphism of FIG. 1 or a complement thereof, wherein the amplified polynucleotide may be greater than about 16 nucleotides in length. The polymorphism may be in linkage disequilibrium with a polymorphism of FIG. 1. The polymorphism may also be selected from the polymorphisms of FIG. 1.

### Isolated Nucleic Acid Molecules and SNP Detection Reagents & Kits

FIG. 1 provides information identifying the SNPs that may be associated with DDD. FIG. 1 includes additional information about the SNP, such as nucleotide substitution, chromosome number, cytogenetic band and p-values from the current disclosure, as well as the genomic-based SNP context sequences. In some embodiments, the context sequences generally include approximately 25 nucleotides upstream (5′) plus 25 nucleotides downstream (3′) of each SNP position, and the alternative nucleotides (alleles) at each SNP position.

### Isolated Nucleic Acid Molecules

The present disclosure further provides isolated polynucleotide molecules that specifically hybridize to a polynucleotide molecule containing the nucleotide sequence of a polymorphism selected from any one of the polymorphisms of FIG. 1 or a polymorphism that may be in linkage disequilibrium with a polymorphism of FIG. 1 or a complement thereof. In some embodiments, the polymorphism may be in linkage disequilibrium with a polymorphism of FIG. 1. In some embodiments, the polymorphism may be selected from the polymorphisms of FIG. 1.

In some embodiments, the isolated polynucleotides may be from about 8-70 nucleotides in length.

In some embodiments the polynucleotide may be an allele-specific probe. In some embodiments, the polynucleotide may be an allele-specific primer.

The present disclosure provides isolated nucleic acid molecules that contain one or more SNPs disclosed in FIG. 1. Isolated nucleic acid molecules contain one or more SNPs identified in FIG. 1. Isolated nucleic acid molecules containing one or more SNPs disclosed in FIG. 1 may be interchangeably referred to throughout the present text as “SNP-containing nucleic acid molecules.” The isolated nucleic acid molecules also include probes and primers (which may be described in greater detail below in the section entitled “SNP Detection Reagents”), which may be used for assaying the disclosed SNPs, and isolated full-length genes, transcripts, cDNA molecules, and fragments thereof, which may be used for such purposes as expressing an encoded protein.

As used herein, an “isolated nucleic acid molecule” can be one that contains a SNP or one that hybridizes to such molecule such as a nucleic acid with a complementary sequence, and may be separated from most other nucleic acids present in the natural source of the nucleic acid molecule. Moreover, an “isolated” nucleic acid molecule, such as a cDNA molecule containing a SNP, can be substantially free of other cellular material, or culture medium when produced by recombinant techniques, or chemical precursors or other chemicals when chemically synthesized. A nucleic acid molecule can be fused to other coding or regulatory sequences and still be considered “isolated.” Nucleic acid molecules present in non-human transgenic animals, which may not naturally occur in the animal, may also be considered “isolated.” Recombinant DNA molecules contained in a vector may be considered “isolated.” Further examples of “isolated” DNA molecule\es include recombinant DNA molecules maintained in heterologous host cells, and purified (partially or substantially) DNA molecules in solution. Isolated RNA molecules include in vivo or in vitro RNA transcripts of the isolated SNP-containing DNA molecules. Isolated nucleic acid molecules according to the present disclosure further include such molecules produced synthetically.

Generally, an isolated SNP-containing nucleic acid molecule comprises one or more SNP positions disclosed by the present disclosure with flanking nucleotide sequences on either side of the SNP positions. A flanking genomic context sequence can include nucleotide residues that may be naturally associated with the SNP site and/or heterologous nucleotide sequences. The flanking sequence may be up to about: 100, 60, 50, 30, 25, 20, 15, 10, 8, or 4 nucleotides (or any other length in-between) on either side of a SNP position.

For full-length genes and entire protein-coding sequences, a SNP flanking sequence can be up to about: 5 KB, 4 KB, 3 KB, 2 KB, or 1 KB on either side of the SNP. Furthermore, in such instances, the isolated nucleic acid molecule can comprise exonic sequences (including protein-coding and/or non-coding exonic sequences), but may also include intronic sequences. Thus, any protein coding sequence may be either contiguous or separated by introns. In some embodiments, the nucleic acid may be isolated from remote and unimportant flanking sequences and may be of appropriate length such that it can be subjected to the specific manipulations or uses described herein such as recombinant protein expression, preparation of probes and primers for assaying the SNP position, and other uses specific to the SNP-containing nucleic acid sequences.

An isolated SNP-containing nucleic acid molecule can comprise a full-length gene or transcript, such as a gene isolated from genomic DNA (e.g., by cloning or PCR amplification), a cDNA molecule, or an mRNA transcript molecule. Furthermore, fragments of such full-length genes and transcripts that contain one or more SNPs may also be encompassed by the present disclosure, and such fragments may be used to express any part of a protein, such as a particular functional domain or an antigenic epitope.

Thus, the present disclosure also encompasses fragments of the nucleic acid sequences provided in FIG. 1, contiguous nucleotide sequence at least about 8 or more nucleotides, in some embodiments, at least about 12 or more nucleotides, and in some embodiments at least about 16 or more nucleotides. Further, a fragment may comprise at least about: 18, 20, 22, 25, 30, 40, 50, 60, 100, 250 or 500 (or any other number in-between) nucleotides in length. The length of the fragment may be based on its intended use. The fragment can be useful as a polynucleotide probe or primer. Such fragments can be isolated using the nucleotide sequences provided in FIG. 1 for the synthesis of a polynucleotide probe. A labeled probe can then be used to screen a cDNA library, genomic DNA library, or mRNA to isolate nucleic acid corresponding to the coding region. Further, primers can be used in amplification reactions, such as for purposes of assaying one or more SNPs sites or for cloning specific regions of a gene.

In some embodiments, an isolated nucleic acid molecule can further encompass a SNP-containing polynucleotide that may be the product of any one of a variety of nucleic acid amplification methods, which may be used to increase the copy numbers of a polynucleotide of interest in a nucleic acid sample. Such amplification methods may include polymerase chain reaction (PCR), ligase chain reaction (LCR), strand displacement amplification (SDA), transcription-mediated amplification (TMA), linked linear amplification (LLA), and the like, and isothermal amplification methods such as nucleic acid sequence based amplification (NASBA), and self-sustained sequence replication. Based on such methodologies, a person skilled in the art can readily design primers in any suitable regions 5′ and 3′ to a SNP. Such primers may be used to amplify DNA of any length so long that it contains the SNP of interest in its sequence.

As used herein, an “amplified polynucleotide” of the disclosure may be a SNP-containing nucleic acid molecule whose amount has been increased at least two fold by any nucleic acid amplification method performed in vitro as compared to its starting amount in a test sample. In some embodiments, an amplified polynucleotide may be the result of at least ten fold, fifty fold, one hundred fold, one thousand fold, or even ten thousand fold increase as compared to its starting amount in a test sample. In a PCR amplification, a polynucleotide of interest may be often amplified at least fifty thousand fold in amount over the unamplified genomic DNA, but the precise amount of amplification needed for an assay depends on the sensitivity of the subsequent detection method used.

Generally, an amplified polynucleotide may be at least about 16 nucleotides in length. In some embodiments, an amplified polynucleotide may be at least about 20 nucleotides in length. In some embodiments, an amplified polynucleotide may be at least about 30 nucleotides in length. In some embodiments, an amplified polynucleotide may be at least about: 32, 40, 45, 50, or 60 nucleotides in length. In some embodiments, an amplified polynucleotide may be at least about: 100, 200, or 300 nucleotides in length. While the total length of an amplified polynucleotide of the disclosure can be as long as an exon, an intron or the entire gene where the SNP of interest resides, an amplified product may be no greater than about 1,000 nucleotides in length (although certain amplification methods may generate amplified products greater than about 1000 nucleotides in length). In some embodiments, an amplified polynucleotide may not be greater than about 600 nucleotides in length. It may be understood that irrespective of the length of an amplified polynucleotide, a SNP of interest may be located anywhere along its sequence.

In a specific embodiment of the disclosure, the amplified product may be at least about 201 nucleotides in length, and/or comprises one of the nucleotide sequences shown in FIG. 1. Such a product may have additional sequences on its 5′ end or 3′ end or both. In some embodiments, the amplified product may be about 101 nucleotides in length, and contains a SNP. Generally, the SNP may be located at the middle of the amplified product (e.g., at position 101 in an amplified product that may be 201 nucleotides in length, or at position 51 in an amplified product that may be 101 nucleotides in length), or within about: 1, 2, 3, 4, 5, 6, 7, 8, 9, 10, 12, 15, or 20 nucleotides from the middle of the amplified product (however, as indicated above, the SNP of interest may be located anywhere along the length of the amplified product).

The present disclosure provides isolated nucleic acid molecules that comprise, consist of, or consist essentially of one or more polynucleotide sequences that contain one or more SNPs, complements thereof, and SNP-containing fragments thereof.

Accordingly, the present disclosure provides nucleic acid molecules that consist of any of the nucleotide sequences shown in FIG. 1. A nucleic acid molecule consists of a nucleotide sequence when the nucleotide sequence may be the complete nucleotide sequence of the nucleic acid molecule.

The present disclosure further provides nucleic acid molecules that consist essentially of any of the nucleotide sequences shown in FIG. 1. A nucleic acid molecule consists essentially of a nucleotide sequence when such a nucleotide sequence may be present with only a few additional nucleotide residues in the final nucleic acid molecule.

The present disclosure further provides nucleic acid molecules that comprise any of the nucleotide sequences shown in FIG. 1. A nucleic acid molecule comprises a nucleotide sequence when the nucleotide sequence may be at least part of the final nucleotide sequence of the nucleic acid molecule. In such a fashion, the nucleic acid molecule can be only the nucleotide sequence or have additional nucleotide residues, such as residues that are naturally associated with it or heterologous nucleotide sequences. Such a nucleic acid molecule can have one to a few additional nucleotides or can comprise many more additional nucleotides.

Isolated nucleic acid molecules can be in the form of RNA, such as mRNA, or in the form DNA, including cDNA and genomic DNA, which may be obtained by molecular cloning or produced by chemical synthetic techniques or by a combination thereof (Sambrook and Russell, 2000, Molecular Cloning: A Laboratory Manual, Cold Spring Harbor Press, N.Y.). Furthermore, isolated nucleic acid molecules, particularly SNP detection reagents such as probes and primers, can also be partially or completely in the form of one or more types of nucleic acid analogs, such as peptide nucleic acid (PNA) (U.S. Pat. Nos. 5,539,082; 5,527,675; 5,623,049; 5,714,331). The nucleic acid, especially DNA, can be double-stranded or single-stranded. Single-stranded nucleic acid can be the coding strand (sense strand) or the complementary non-coding strand (anti-sense strand). DNA, RNA, or PNA segments can be assembled from fragments of the human genome (in the case of DNA or RNA) or single nucleotides, short oligonucleotide linkers, or from a series of oligonucleotides, to provide a synthetic nucleic acid molecule. Nucleic acid molecules can be readily synthesized using the sequences provided herein as a reference;

The present disclosure encompasses nucleic acid analogs that contain modified, synthetic, or non-naturally occurring nucleotides or structural elements or other alternative/modified nucleic acid chemistries. Such nucleic acid analogs may be utilized as detection reagents (e.g., primers/probes) for detecting one or more SNPs identified in FIG. 1. Furthermore, kits/systems (such as beads, arrays, etc.) that include these analogs may be encompassed by the present disclosure.

Additional examples of nucleic acid modifications that improve the binding properties and/or stability of a nucleic acid include the use of base analogs such as inosine, intercalators (U.S. Pat. No. 4,835,263) and the minor groove binders (U.S. Pat. No. 5,801,115). Thus, references herein to nucleic acid molecules, SNP-containing nucleic acid molecules, SNP detection reagents (e.g., probes and primers), and oligonucleotides/polynucleotides include PNA oligomers and other nucleic acid analogs.

Further variants of the nucleic acid molecules disclosed in FIG. 1, such as naturally occurring allelic variants (as well as orthologs and paralogs) and synthetic variants produced by mutagenesis techniques, can be identified and/or produced. Such further variants can comprise a nucleotide sequence that shares at least about: 70-80%, 80-85%, 85-90%, 91%, 92%, 93%, 94%, 95%, 96%, 97%, 98%, or 99% sequence identity with a nucleic acid sequence disclosed in FIG. 1 (or a fragment thereof) and that includes a novel SNP allele disclosed in FIG. 1. Thus, the present disclosure specifically contemplates isolated nucleic acid molecule that have a certain degree of sequence variation compared with the sequences shown in FIG. 1, but that contain a novel SNP allele. In other words, as long as an isolated nucleic acid molecule contains a novel SNP allele, other portions of the nucleic acid molecule that flank the novel SNP allele can vary to some degree from the specific genomic and context sequences shown in FIG. 1.

To determine the percent identity of two nucleotide sequences of two molecules that share sequence homology, the sequences may be aligned for optimal comparison purposes (e.g., gaps can be introduced in one or both of a first and a second nucleic acid sequence for optimal alignment and non-homologous sequences can be disregarded for comparison purposes). In some embodiments, at least about: 30%, 40%, 50%, 60%, 70%, 80%, or 90% or more of the length of a reference sequence may be aligned for comparison purposes. The nucleotides at corresponding nucleotide positions may be compared. When a position in the first sequence may be occupied by the same nucleotide as the corresponding position in the second sequence, then the molecules may be identical at that position (as used herein, nucleic acid “identity” may be equivalent to nucleic acid “homology”). The percent identity between the two sequences may be a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm.

In some embodiments, the percent identity between two nucleotide sequences may be determined using the GAP program in the GCG software package, using an NWSgapdna. CMP matrix and a gap weight of about: 40, 50, 60, 70, or 80 and a length weight of about: 1, 2, 3, 4, 5, or 6. In some embodiments, the percent identity between two nucleotide sequences may be determined using the algorithm of E. Myers and W. Miller which has been incorporated into the ALIGN program (version 2.0), using a PAM120 weight residue table, a gap length penalty of 12, and a gap penalty of 4.

The nucleotide sequences can be further used as a “query sequence” to perform a search against sequence databases to identify other family members or related sequences. Such searches can be performed using the NBLAST and XBLAST programs (version 2.0). BLAST nucleotide searches can be performed with the NBLAST program, score=100, wordlength=12 to obtain nucleotide sequences homologous to the nucleic acid molecules of the disclosure. To obtain gapped alignments for comparison purposes, Gapped BLAST can be utilized. When utilizing BLAST and gapped BLAST programs, the default parameters of the respective programs (e.g., XBLAST and NBLAST) can be used. In addition to BLAST, examples of other search and sequence comparison programs may include FASTA and KERR.

### SNP Detection Reagents

In some embodiments, the SNPs can be used for the design of SNP detection reagents. As used herein, a “SNP detection reagent” may be a reagent that specifically detects a specific target SNP position, and that may be specific for a particular nucleotide (allele) of the target SNP position (i.e., the detection reagent, in some embodiments, can differentiate between different alternative nucleotides at a target SNP position, thereby allowing the identity of the nucleotide present at the target SNP position to be determined). In some embodiments, such detection reagent hybridizes to a target SNP-containing nucleic acid molecule by complementary base-pairing in a sequence specific manner, and discriminates the target variant sequence from other nucleic acid sequences. An example of a detection reagent may be a probe that hybridizes to a target nucleic acid containing one or more of the SNPs. In some embodiments, such a probe can differentiate between nucleic acids having a particular nucleotide (allele) at a target SNP position from other nucleic acids that have a different nucleotide at the same target SNP position. In addition, a detection reagent may hybridize to a specific region 5′ and/or 3′ to a SNP position, particularly a region corresponding to the context sequences provided in the SNPs. Another example of a detection reagent may be a primer which acts as an initiation point of nucleotide extension along a complementary strand of a target polynucleotide. The SNP sequence information provided herein may be useful for designing primers, e.g. allele-specific primers, to amplify (e.g., using PCR) any SNP.

In some embodiments, a SNP detection reagent can be a synthetic polynucleotide molecule, such as an isolated or synthetic DNA or RNA polynucleotide probe or primer or PNA oligomer, or a combination of DNA, RNA and/or PNA that hybridizes to a segment of a target nucleic acid molecule containing a SNP identified herein. A detection reagent in the form of a polynucleotide may contain modified base analogs, intercalators or minor groove binders. Multiple detection reagents such as probes may be affixed to a solid support (e.g., arrays or beads) or supplied in solution (e.g., probe/primer sets for enzymatic reactions such as PCR, RT-PCR, TaqMan assays, or primer-extension reactions) to form a SNP detection kit.

A probe or primer may be a substantially purified oligonucleotide. Such oligonucleotide may comprise a region of complementary nucleotide sequence that hybridizes under stringent conditions to at least about: 8, 10, 12, 16, 18, 20, 22, 25, 30, 40, 50, 60, 100 (or any other number in-between) or more consecutive nucleotides in a target nucleic acid molecule. Depending on the particular assay, the consecutive nucleotides can either include the target SNP position, or be a specific region in close enough proximity 5′ and/or 3′ to the SNP position to carry out the desired assay.

Other primer and probe sequences can readily be determined using the nucleotide sequences. It may be apparent to one of skill in the art that such primers and probes may be directly useful as reagents for genotyping the SNPs, and can be incorporated into any kit/system format.

In order to produce a probe or primer specific for a target SNP-containing sequence, the gene/transcript and/or context sequence surrounding the SNP of interest may be examined using a computer algorithm which starts at the 5′ or at the 3′ end of the nucleotide sequence. Algorithms may then identify oligomers of defined length that may be unique to the gene/SNP context sequence, have a GC content within a range suitable for hybridization, lack predicted secondary structure that may interfere with hybridization, and/or possess other desired characteristics or that lack other undesired characteristics.

A primer or probe may be at least about 8 nucleotides in length. In one embodiment of the disclosure, a primer or a probe may be at least about 10 nucleotides in length. In some embodiments, a primer or a probe may be at least about 12 nucleotides in length. In some embodiments, a primer or probe may be at least about 16, 17, 18, 19, 20, 21, 22, 23, 24 or 25 nucleotides in length. While the maximal length of a probe can be as long as the target sequence to be detected, depending on the type of assay in which it may be employed, it may be less than about 50, 60, 65, or 70 nucleotides in length. In the case of a primer, it may be less than about 30 nucleotides in length. In some embodiments, a primer or a probe may be within the length of about 18 and about 28 nucleotides. However, in some embodiments, such as nucleic acid arrays and in some embodiments in which probes may be affixed to a substrate, the probes can be longer, such as on the order of from about 30 to about 70, or about: 75, 80, 90, 100, or more nucleotides in length (see the section below entitled “SNP Detection Kits and Systems”).

For analyzing SNPs, it may be appropriate to use oligonucleotides specific for alternative SNP alleles. Such oligonucleotides which detect single nucleotide variations in target sequences may be referred to by such terms as “allele-specific oligonucleotides”, “allele-specific probes”, or “allele-specific primers”.

While the design of each allele-specific primer or probe depends on variables such as the precise composition of the nucleotide sequences flanking a SNP position in a target nucleic acid molecule, and the length of the primer or probe, another factor in the use of primers and probes may be the stringency of the condition under which the hybridization between the probe or primer and the target sequence may be performed. Higher stringency conditions utilize buffers with lower ionic strength and/or a higher reaction temperature, and may require a more perfect match between probe/primer and a target sequence in order to form a stable duplex. If the stringency may be too high, however, hybridization may not occur at all. In contrast, lower stringency conditions utilize buffers with higher ionic strength and/or a lower reaction temperature, and permit the formation of stable duplexes with more mismatched bases between a probe/primer and a target sequence. By way of example and not limitation, exemplary conditions for high stringency hybridization conditions using an allele-specific probe may be as follows: Prehybridization with a solution containing 5× standard saline phosphate EDTA (SSPE), 0.5% NaDodSO4 (SDS) at 55° C., and incubating probe with target nucleic acid molecules in the same solution at the same temperature, followed by washing with a solution containing 2×SSPE, and 0.1% SDS at 55° C. or room temperature.

Moderate stringency hybridization conditions may be used for allele-specific primer extension reactions with a solution containing, e.g., about 50 mM KCl at about 46° C. Alternatively, the reaction may be carried out at an elevated temperature such as 60° C. In some embodiments, a moderately stringent hybridization condition suitable for oligonucleotide ligation assay (OLA) reactions wherein two probes may be ligated if they are complementary to the target sequence may utilize a solution of about 100 mM KCl at a temperature of 46° C.

In a hybridization-based assay, allele-specific probes can be designed that hybridize to a segment of target DNA from one individual but may not hybridize to the corresponding segment from another individual due to the presence of different polymorphic forms (e.g., alternative SNP alleles/nucleotides) in the respective DNA segments from the two individuals. Hybridization conditions may be sufficiently stringent that there may be a significant detectable difference in hybridization intensity between alleles and in some embodiments an essentially binary response, whereby a probe hybridizes to only one of the alleles or significantly more strongly to one allele. While a probe may be designed to hybridize to a target sequence that contains a SNP site such that the SNP site aligns anywhere along the sequence of the probe, the probe may be designed to hybridize to a segment of the target sequence such that the SNP site aligns with a central position of the probe (e.g., a position within the probe that may be at least three nucleotides from either end of the probe). This design of probe generally achieves good discrimination in hybridization between different allelic forms.

In some embodiments, a probe or primer may be designed to hybridize to a segment of target DNA such that the SNP aligns with either the 5′ most end or the 3′ most end of the probe or primer. In some embodiments suitable for use in an oligonucleotide ligation assay (U.S. Pat. No. 4,988,617), the most 3′ nucleotide of the probe aligns with the SNP position in the target sequence.

Oligonucleotide probes and primers may be prepared. Chemical synthetic methods may include the phosphotriester method; the phosphodiester method, the diethylphosphoamidate method; and the solid support method.

Allele-specific probes may be often used in pairs (or, in sets of 3 or 4, such as if a SNP position has 3 or 4 alleles, respectively, or to assay both strands of a nucleic acid molecule for a target SNP allele), and such pairs may be identical except for a one nucleotide mismatch that represents the allelic variants at the SNP position. In some cases, one member of a pair perfectly matches a reference form of a target sequence that has a SNP allele (i.e., the allele that may be more frequent in the target population) and the other member of the pair perfectly matches a form of the target sequence that has a SNP allele (i.e., the allele that may be rarer in the target population). In the case of an array, multiple pairs of probes can be immobilized on the same support for simultaneous analysis of multiple different polymorphisms.

In one type of PCR-based assay, an allele-specific primer hybridizes to a region on a target nucleic acid molecule that overlaps a SNP position and only primes amplification of an allelic form to which the primer exhibits perfect complementarity. In some embodiments, the primer's 3′-most nucleotide may be aligned with and complementary to the SNP position of the target nucleic acid molecule. This primer may be used in conjunction with a second primer that hybridizes at a distal site. Amplification may proceed from the two primers, producing a detectable product that indicates which allelic form may be present in the test sample. A control may be usually performed with a second pair of primers, one of which may show a single base mismatch at the polymorphic site and the other of which exhibits perfect complementarity to a distal site. The single-base mismatch may prevent amplification or substantially reduces amplification efficiency, so that either no detectable product may be formed or it may be formed in lower amounts or at a slower pace. In some embodiments, the method may generally work most effectively when the mismatch may be at the 3′-most position of the oligonucleotide (i.e., the 3′-most position of the oligonucleotide aligns with the target SNP position) because this position may be most destabilizing to elongation from the primer (see, e.g., WO 93/22456). This PCR-based assay can be utilized as part of the TaqMan assay, described below.

In a specific embodiment of the disclosure, a primer of the disclosure contains a sequence substantially complementary to a segment of a target SNP-containing nucleic acid molecule except that the primer has a mismatched nucleotide in one of the three nucleotide positions at the 3′-most end of the primer, such that the mismatched nucleotide does not base pair with a particular allele at the SNP site. In some embodiments, the mismatched nucleotide in the primer may be the second from the last nucleotide at the 3′-most position of the primer. In some embodiments, the mismatched nucleotide in the primer may be the last nucleotide at the 3′-most position of the primer.

In some embodiments, a SNP detection reagent of the disclosure may be labeled with a fluorogenic reporter dye that emits a detectable signal. While a reporter dye may be a fluorescent dye, any reporter dye that can be attached to a detection reagent such as an oligonucleotide probe or primer may be suitable for use in the disclosure. Such dyes may include Acridine, AMCA, BODIPY, Cascade Blue, Cy2, Cy3, Cy5, Cy7, Dabcyl, Edans, Eosin, Erythrosin, Fluorescein, 6-Fam, Tet, Joe, Hex, Oregon Green, Rhodamine, Rhodol Green, Tamra, Rox, and Texas Red.

In yet another embodiment of the disclosure, the detection reagent may be further labeled with a quencher dye such as Tamra, especially when the reagent may be used as a self-quenching probe such as a TaqMan (U.S. Pat. Nos. 5,210,015 and 5,538,848) or Molecular Beacon probe (U.S. Pat. Nos. 5,118,801 and 5,312,728), or other stemless or linear beacon probe.

The detection reagents of the disclosure may also contain other labels and may include biotin for streptavidin binding and oligonucleotide for binding to another complementary oligonucleotide such as pairs of zipcodes.

The present disclosure also contemplates reagents that may not contain (or that may be complementary to) a SNP nucleotide identified herein but that may be used to assay one or more SNPs. Primers that flank, but may not hybridize directly to a target SNP position, may be useful in primer extension reactions in which the primers hybridize to a region adjacent to the target SNP position (i.e., within one or more nucleotides from the target SNP site). During the primer extension reaction, a primer may be not able to extend past a target SNP site if a particular nucleotide (allele) may be present at that target SNP site, and the primer extension product can readily be detected in order to determine which SNP allele may be present at the target SNP site. Particular ddNTPs may be used in the primer extension reaction to terminate primer extension once a ddNTP may be incorporated into the extension product (a primer extension product which includes a ddNTP at the 3′-most end of the primer extension product, and in which the ddNTP corresponds to a SNP, may be a composition that may be encompassed by the present disclosure). Thus, in some embodiments, reagents that bind to a nucleic acid molecule in a region adjacent to a SNP site, even though the bound sequences may not necessarily include the SNP site itself, may be encompassed by the present disclosure.

### SNP Detection Kits and Systems

In some embodiments, based on a variant such as SNP or indels and associated sequence information, detection reagents can be developed and used to assay any variant individually or in combination, and such detection reagents can be readily incorporated into a kit or system formats. The terms “kits” and “systems” can refer to such things as combinations of multiple variant detection reagents, or one or more variant detection reagents in combination with one or more other types of elements or components (e.g., other types of biochemical reagents, containers, packages such as packaging intended for commercial sale, substrates to which variant detection reagents may be attached, electronic hardware components, etc.). Accordingly, the present disclosure further provides variant detection kits and systems that may include packaged probe and primer sets (e.g., TaqMan probe/primer sets), arrays/microarrays of nucleic acid molecules, and beads that contain one or more probes, primers, or other detection reagents for detecting one or more variants. The kits/systems can include various electronic hardware components; including arrays (“DNA chips”) and microfluidic systems (“lab-on-a-chip” systems) provided by various manufacturers may comprise hardware components. Other kits/systems (e.g., probe/primer sets) may not include electronic hardware components, but may be comprised of one or more variant detection reagents (along with other biochemical reagents) packaged in one or more containers.

In some embodiments, provided herein may be a kit comprising one or more variant detection agents, and methods for detecting the variants by employing detection reagents. In some embodiments, a questionnaire of non-genetic clinical factors may be included. In some embodiments, provided herein may be a method of identifying an individual having an increased or decreased risk of developing degenerative disc disease by detecting the presence or absence of a variant allele. In some embodiments, provided herein may be a method for diagnosis of degenerative disc disease by detecting the presence or absence of a variant allele. In some embodiments, provided herein may be a method for predicting degenerative disc disease sub-classification by detecting the presence or absence of a variant allele. In some embodiments, the questionnaire may be completed by a subject or by another individual (such as a medical professional, including a social worker, health worker, doctor, technician, medical intake professional) based on medical history physical exam or other clinical findings. In some embodiments, the questionnaire may include any other non-genetic clinical factors known to be associated with the risk of developing degenerative disc disease. In some embodiments, a reagent for detecting a variant in the context of its naturally-occurring flanking nucleotide sequences (which can be, e.g., either DNA or mRNA) is provided. In some embodiments, the reagent may be in the form of a hybridization probe or an amplification primer that is useful in the specific detection of a variant of interest. In some embodiments, a variant can be a genetic polymorphism having a Minor Allele Frequency (MAF) of at least 1% in a population (such as for instance the Caucasian population or the CEU population) and an RV may be understood to be a genetic polymorphism having a Minor Allele Frequency (MAF) of less than 1% in a population (such as for instance the Caucasian population or the CEU population).

In some embodiments, a detection kit can contain one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a variant-containing nucleic acid molecule. A kit may further contain means for determining the amount of a target nucleic acid, and means for comparing the amount with a standard, and can comprise instructions for using the kit to detect the variant-containing nucleic acid molecule of interest. In some embodiments, kits are provided which may contain the necessary reagents to carry out one or more assays to detect one or more variants. In some embodiments, the detection kits/systems can be in the form of nucleic acid arrays, or compartmentalized kits, including microfluidic/lab-on-a-chip systems.

In some embodiments, variant detection kits/systems may contain one or more probes, or pairs of probes, that hybridize to a nucleic acid molecule at or near each target variant position. Multiple pairs of allele-specific probes may be included in the kit/system to simultaneously assay large numbers of variants, at least one of which may be a variant. In some kits/systems, the allele-specific probes may be immobilized to a substrate such as an array or bead. The same substrate can comprise allele-specific probes for detecting at least 1; 10; 100; 1000; 10,000; 100,000; 500,000 (or any other number in-between) or substantially all of the variants.

The terms “arrays,” “microarrays,” and “DNA chips” are used herein interchangeably and may refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate.

In some embodiments, any number of probes, such as allele-specific probes, may be implemented in an array, and each probe or pair of probes can hybridize to a different variant position. In the case of polynucleotide probes, they can be synthesized at designated areas (or synthesized separately and then affixed to designated areas) on a substrate using a light-directed chemical process. Each DNA chip can contain thousands to millions of individual synthetic polynucleotide probes arranged in a grid-like pattern and miniaturized (e.g., to the size of a dime). For example, probes may be attached to a solid support in an ordered, addressable array.

In some embodiments, a microarray can be composed of a large number of unique, single-stranded polynucleotides fixed to a solid support. Polynucleotides may be for example from about 6 to about 60 nucleotides in length, for example from about 15 to about 30 nucleotides in length, for example from about 18 to about 25 nucleotides in length. For certain types of microarrays or other detection kits/systems, it may be suitable to use oligonucleotides that may be from about 7 to about 20 nucleotides in length. In other types of arrays, such as arrays used in conjunction with chemiluminescent detection technology, exemplary probe lengths can be from about 15 to about 80 nucleotides in length, for example from about 50 to about 70 nucleotides in length, example from about 55 to about 65 nucleotides in length, and for example about 60 nucleotides in length. The microarray or detection kit can contain polynucleotides that cover the known 5′ or 3′ sequence of the target variant site, sequential polynucleotides that cover the full-length sequence of a gene/transcript, or unique polynucleotides selected from particular areas along the length of a target gene/transcript sequence, particularly areas corresponding to one or more variants. Polynucleotides used in the microarray or detection kit can be specific to a variant or variants of interest (e.g., specific to a particular SNP allele at a target SNP site, or specific to particular SNP alleles at multiple different SNP sites), or specific to a polymorphic gene/transcript or genes/transcripts of interest.

In some embodiments, hybridization assays based on polynucleotide arrays rely on the differences in hybridization stability of the probes to perfectly matched and mismatched target sequence variants. For variant genotyping, it may be generally suitable that stringency conditions used in hybridization assays may be high enough such that nucleic acid molecules that differ from one another at as little as a single variant position can be differentiated (e.g., variant hybridization assays may be designed so that hybridization may occur only if one particular nucleotide may be present at a variant position, but may not occur if an alternative nucleotide may be present at that variant position). Such high stringency conditions may be suitable when using nucleic acid arrays of allele-specific probes for variant detection. In some embodiments, the arrays may be used in conjunction with chemiluminescent detection technology.

In some embodiments, a nucleic acid array can comprise an array of probes of from about 15 to about 25 nucleotides in length. In some embodiments, a nucleic acid array can comprise any number of probes, in which at least one probe may be capable of detecting one or more variants and/or at least one probe comprises a fragment of one of the sequences selected from the group consisting of those, and sequences complementary thereto, said fragment comprising at least about 8 consecutive nucleotides, for example about: 10, 11, 12, 13, 14, 15, 16, 17, 18, 19, 20, or more for example about: 22, 25, 30, 40, 47, 50, 55, 60, 65, 70, 80, 90, 100, or more consecutive nucleotides (or any other number in-between) and containing (or being complementary to) a variant. In some embodiments, the nucleotide complementary to the variant site may be within about: 5, 4, 3, 2, or 1 nucleotide from the center of the probe, for example at the center of said probe.

In some embodiments, using such arrays or other kits/systems, the present disclosure provides methods of identifying the variants in a test sample. Such methods may involve incubating a test sample of nucleic acids with an array comprising one or more probes corresponding to at least one variant position, and assaying for binding of a nucleic acid from the test sample with one or more of the probes. Conditions for incubating a variant detection reagent (or a kit/system that employs one or more such variant detection reagents) with a test sample vary. Incubation conditions depend on such factors as the format employed in the assay, the detection methods employed, and the type and nature of the detection reagents used in the assay. One skilled in the art may recognize that any one of the hybridization, amplification and array assay formats can readily be adapted to detect the variants.

In some embodiments, a detection kit/system may include components that may be used to prepare nucleic acids from a test sample for the subsequent amplification and/or detection of a variant-containing nucleic acid molecule. Such sample preparation components can be used to produce nucleic acid extracts, including DNA and/or RNA, extracts from any bodily fluids. In an exemplary embodiment of the disclosure, the bodily fluid may be blood, saliva or buccal swabs. The test samples used in the above-described methods may vary based on such factors as the assay format, nature of the detection method, and the specific tissues, cells or extracts used as the test sample to be assayed. Methods of preparing nucleic acids can be readily adapted to obtain a sample that may be compatible with the system utilized. In some embodiments, in addition to reagents for preparation of nucleic acids and reagents for detection of one of the variants of this disclosure, the kit may include a questionnaire inquiring about non-genetic clinical factors such as age, gender, or any other non-genetic clinical factors known to be associated with degenerative disc disease.

In some embodiments, a form of kit can be a compartmentalized kit. A compartmentalized kit includes any kit in which reagents may be contained in separate containers. Such containers may include small glass containers, plastic containers, strips of plastic, glass or paper, or arraying material such as silica. Such containers allow one to efficiently transfer reagents from one compartment to another compartment such that the test samples and reagents may not be cross-contaminated, or from one container to another vessel not included in the kit, and the agents or solutions of each container can be added in a quantitative fashion from one compartment to another or to another vessel. Such containers may include one or more containers which may accept the test sample, one or more containers which contain at least one probe or other variant detection reagent for detecting one or more variants, one or more containers which contain wash reagents (such as phosphate buffered saline, Tris-buffers, etc.), and one or more containers which contain the reagents used to reveal the presence of the bound probe or other variant detection reagents. The kit can further comprise compartments and/or reagents for nucleic acid amplification or other enzymatic reactions such as primer extension reactions, hybridization, ligation, electrophoresis (for example capillary electrophoresis), mass spectrometry, and/or laser-induced fluorescent detection. The kit may also include instructions for using the kit. In such microfluidic devices, the containers may be referred to as microfluidic “compartments”, “chambers”, or “channels”.

In some embodiments, microfluidic devices, which may also be referred to as “lab-on-a-chip” systems, biomedical micro-electro-mechanical systems (bioMEMs), or multicomponent integrated systems, may be exemplary kits/systems for analyzing variants. Such systems miniaturize and compartmentalize processes such as probe/target hybridization, nucleic acid amplification, and capillary electrophoresis reactions in a single functional device. Such microfluidic devices may utilize detection reagents in at least one aspect of the system, and such detection reagents may be used to detect one or more variants. One example of a microfluidic system may be the integration of PCR amplification and capillary electrophoresis in chips. Exemplary microfluidic systems comprise a pattern of microchannels designed onto a glass, silicon, quartz, or plastic wafer included on a microchip. The movements of the samples may be controlled by electric, electroosmotic or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip. In some embodiments, for genotyping variants, a microfluidic system may integrate nucleic acid amplification, primer extension, capillary electrophoresis, and a detection method such as laser induced fluorescence detection.

A person skilled in the art may recognize that, based on the SNP and associated sequence information, detection reagents can be developed and used to assay any SNP individually or in combination, and such detection reagents can be readily incorporated into one of the established kit or system formats.

The kits may be used for detecting a nucleic acid polymorphism indicative of an altered risk in a symptomatic or presymptomatic DDD subject. Such kits may comprise a polynucleotide having a SNP of FIG. 1, a SNP that may be in linkage disequilibrium with a SNP of FIG. 1 or a SNP of FIG. 1, enzymes, buffers, and reagents used to detect genetic polymorphisms. The kits may further comprise a questionnaire of non-genetic clinical factors. In other embodiments, a kit may comprise a probe that binds to a SNP of FIG. 1.

The terms “kits” and “systems”, as used herein in the context of SNP detection reagents, may be intended to refer to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents may be attached, electronic hardware components, etc.). Accordingly, the present disclosure further provides SNP detection kits and systems that may include packaged probe and primer sets (e.g., TaqMan probe/primer sets), arrays/microarrays of nucleic acid molecules, and beads that contain one or more probes, primers, or other detection reagents for detecting one or more SNPs. The kits/systems can include various electronic hardware components; for example, arrays (“DNA chips”) and microfluidic systems (“lab-on-a-chip” systems) provided by various manufacturers may comprise hardware components. Other kits/systems (e.g., probe/primer sets) may not include electronic hardware components, but may be comprised of one or more SNP detection reagents (along with other biochemical reagents) packaged in one or more containers.

In some embodiments, a SNP detection kit may contain one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) that may be necessary to carry out an assay or reaction, such as amplification and/or detection of a SNP-containing nucleic acid molecule. A kit may further contain means for determining the amount of a target nucleic acid, and means for comparing the amount with a standard, and can comprise instructions for using the kit to detect the SNP-containing nucleic acid molecule of interest. In some embodiments, kits may be provided which contain the necessary reagents to carry out one or more assays to detect one or more SNPs. In some embodiments, SNP detection kits/systems may be in the form of nucleic acid arrays, or compartmentalized kits, including microfluidic/lab-on-a-chip systems.

SNP detection kits/systems may contain one or more probes, or pairs of probes, that hybridize to a nucleic acid molecule at or near each target SNP position. Multiple pairs of allele-specific probes may be included in the kit/system to simultaneously assay large numbers of SNPs, at least one of which may be a SNP. In some kits/systems, the allele-specific probes may be immobilized to a substrate such as an array or bead. For example, the same substrate can comprise allele-specific probes for detecting at least 1; 10; 100; 1000; 10,000; 100,000; 500,000 (or any other number in-between) or substantially all of the SNPs.

The terms “arrays,” “microarrays,” and “DNA chips” are used herein interchangeably and may refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate.

Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a light-directed chemical process. Each DNA chip can contain thousands to millions of individual synthetic polynucleotide probes arranged in a grid-like pattern and miniaturized (e.g., to the size of a dime). In some embodiments, probes may be attached to a solid support in an ordered, addressable array.

A microarray can be composed of a large number of unique, single-stranded polynucleotides fixed to a solid support. Polynucleotides may be from about 6 to about 60 nucleotides in length, in some embodiments from about 15 to about 30 nucleotides in length, and in some embodiments from about 18 to about 25 nucleotides in length. In some embodiments, for certain types of microarrays or other detection kits/systems, it may be preferable to use oligonucleotides that may be from about 7 to about 20 nucleotides in length. In other types of arrays, such as arrays used in conjunction with chemiluminescent detection technology, a probe length can be from about 15 to about 80 nucleotides in length, from about 50 to about 70 nucleotides in length, from about 55 to about 65 nucleotides in length, and about 60 nucleotides in length. The microarray or detection kit can contain polynucleotides that cover the 5′ or 3′ sequence of the target SNP site, sequential polynucleotides that cover the full-length sequence of a gene/transcript; or unique polynucleotides selected from particular areas along the length of a target gene/transcript sequence, particularly areas corresponding to one or more SNPs. Polynucleotides used in the microarray or detection kit can be specific to a SNP or SNPs of interest (e.g., specific to a particular SNP allele at a target SNP site, or specific to particular SNP alleles at multiple different SNP sites), or specific to a polymorphic gene/transcript or genes/transcripts of interest.

Hybridization assays based on polynucleotide arrays rely on the differences in hybridization stability of the probes to perfectly matched and mismatched target sequence variants. For SNP genotyping, in some embodiments, it may be preferable that stringency conditions used in hybridization assays may be high enough such that nucleic acid molecules that differ from one another at as little as a single SNP position can be differentiated (e.g., SNP hybridization assays may be designed so that hybridization may occur only if one particular nucleotide may be present at a SNP position, but may not occur if an alternative nucleotide may be present at that SNP position). Such high stringency conditions may be, in some embodiments, preferable when using, for example, nucleic acid arrays of allele-specific probes for SNP detection. Such high stringency conditions may be described in the preceding section.

In some embodiments, the arrays may be used in conjunction with chemiluminescent detection technology.

In one embodiment of the disclosure, a nucleic acid array can comprise an array of probes of from about 15 to about 25 nucleotides in length. In some embodiments, a nucleic acid array can comprise any number of probes, in which at least one probe may be capable of detecting one or more SNPs disclosed in FIG. 1 and/or at least one probe comprises a fragment of one of the sequences selected from the group consisting of those, and sequences complementary thereto, said fragment comprising at least about 8 consecutive nucleotides, in some embodiments about: 10, 12, 15, 16, 18, 20, in some embodiments about: 22, 25, 30, 40, 47, 50, 55, 60, 65, 70, 80, 90, 100, or more consecutive nucleotides (or any other number in-between) and containing (or being complementary to) a SNP. In some embodiments, the nucleotide complementary to the SNP site may be within about: 5, 4, 3, 2, or 1 nucleotide from the center of the probe, in some embodiments at the center of said probe.

A polynucleotide probe can be synthesized on the surface of the substrate by using a chemical coupling procedure and an ink jet application apparatus, as described in PCT application WO95/251116 (Baldeschweiler et al.) which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more polynucleotides, or any other number which lends itself to the efficient use of commercially available instrumentation.

Using such arrays or other kits/systems, the present disclosure provides methods of identifying the SNPs in a test sample. Such methods may involve incubating a test sample of nucleic acids with an array comprising one or more probes corresponding to at least one SNP position, and assaying for binding of a nucleic acid from the test sample with one or more of the probes. Conditions for incubating a SNP detection reagent (or a kit/system that employs one or more such SNP detection reagents) with a test sample vary. Incubation conditions depend on such factors as the format employed in the assay, the detection methods employed, and the type and nature of the detection reagents used in the assay. One skilled in the art may recognize that any one of the hybridization, amplification and array assay formats can readily be adapted to detect the SNPs.

A SNP detection kit/system may include components that may be used to prepare nucleic acids from a test sample for the subsequent amplification and/or detection of a SNP-containing nucleic acid molecule. Such sample preparation components can be used to produce nucleic acid extracts, including DNA and/or RNA, extracts from any bodily fluids. In some embodiments, the bodily fluid may be blood, saliva or buccal swabs. The test samples used in the above-described methods may vary based on such factors as the assay format, nature of the detection method, and the specific tissues, cells or extracts used as the test sample to be assayed. Methods of preparing nucleic acids can be readily adapted to obtain a sample that may be compatible with the system utilized.

In yet another form of the kit in addition to reagents for preparation of nucleic acids and reagents for detection of one of the SNPs of this disclosure, the kit may include a questionnaire inquiring about non-genetic clinical factors such as the number of herniated discs, sciatica episodes, decreased disc height, dark nucleus pulposus and the Schneiderman or Pfirrmann grade which evaluates signal changes within the nucleus pulposus of the intervertebral discs of the lumbar spine or any other non-genetic clinical factors associated with DDD.

Another form of kit contemplated by the present disclosure may be a compartmentalized kit. A compartmentalized kit includes any kit in which reagents may be contained in separate containers. Such containers may include small glass containers, plastic containers, strips of plastic, glass or paper, or arraying material such as silica. Such containers allow one to efficiently transfer reagents from one compartment to another compartment such that the test samples and reagents may not be cross-contaminated, or from one container to another vessel not included in the kit, and the agents or solutions of each container can be added in a quantitative fashion from one compartment to another or to another vessel. Such containers may include one or more containers which may accept the test sample, one or more containers which contain at least one probe or other SNP detection reagent for detecting one or more SNPs, one or more containers which contain wash reagents (such as phosphate buffered saline, Tris-buffers, etc.), and one or more containers which contain the reagents used to reveal the presence of the bound probe or other SNP detection reagents. The kit can further comprise compartments and/or reagents for nucleic acid amplification or other enzymatic reactions such as primer extension reactions, hybridization, ligation, electrophoresis (such as capillary electrophoresis), mass spectrometry, and/or laser-induced fluorescent detection. The kit may also include instructions for using the kit. Exemplary compartmentalized kits include microfluidic devices. In such microfluidic devices, the containers may be referred to as microfluidic “compartments”, “chambers”, or “channels”.

Microfluidic devices, which may also be referred to as “lab-on-a-chip” systems, biomedical micro-electro-mechanical systems (bioMEMs), or multicomponent integrated systems, may be exemplary kits/systems for analyzing SNPs. Such systems miniaturize and compartmentalize processes such as probe/target hybridization, nucleic acid amplification, and capillary electrophoresis reactions in a single functional device. Such microfluidic devices may utilize detection reagents in at least one aspect of the system, and such detection reagents may be used to detect one or more SNPs. One example of a microfluidic system may be disclosed in U.S. Pat. No. 5 5,589,136, which describes the integration of PCR amplification and capillary electrophoresis in chips. Exemplary microfluidic system may comprise a pattern of microchannels designed onto a glass, silicon, quartz, or plastic wafer included on a microchip. The movements of the samples may be controlled by electric, electroosmotic or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip. See, for example, U.S. Pat. No. 6,153,073, Dubrow et al., and U.S. Pat. No. 6,156,181, Parce et al.

For genotyping SNPs, a microfluidic system may integrate nucleic acid amplification, primer extension, capillary electrophoresis, and a detection method such as laser induced fluorescence detection.

### Apparatus for Using Nucleic Acid Molecules

In some embodiments, the present disclosure further provides an apparatus for detecting DDD mutations comprising a DNA chip array comprising a plurality of polynucleotides attached to the array, wherein each polynucleotide contains a polymorphism selected from the group consisting of the polymorphisms set forth in FIG. 1 or a polymorphism that may be in linkage disequilibrium with a polymorphism of FIG. 1 or a complement thereof, and a device for detecting the SNPs.

The polymorphism may be selected from the polymorphisms of FIG. 1.

### Uses of Nucleic Acid Molecules

The nucleic acid molecules may have a variety of uses, especially in the diagnosis and treatment of DDD. For example, the nucleic acid molecules may be useful as hybridization probes, such as for genotyping SNPs in messenger RNA, transcript, cDNA, genomic DNA, amplified DNA or other nucleic acid molecules disclosed in FIG. 1 or SNPs disclosed in FIG. 1, as well as their orthologs.

A probe can hybridize to any nucleotide sequence along the entire length of a nucleic acid molecule encompassing a SNP. In some embodiments, a probe hybridizes to a region of a target sequence that encompasses a SNP. In some embodiments, a probe hybridizes to a SNP-containing target sequence in a sequence-specific manner such that it distinguishes the target sequence from other nucleotide sequences which vary from the target sequence only by which nucleotide may be present at the SNP site. Such a probe may be particularly useful for detecting the presence of a SNP-containing nucleic acid in a test sample, or for determining which nucleotide (allele) may be present at a particular SNP site (i.e., genotyping the SNP site).

A nucleic acid hybridization probe may be used for determining the presence, level, form, and/or distribution of nucleic acid expression. The nucleic acid whose level may be determined can be DNA or RNA. Accordingly, probes specific for the SNPs described herein can be used to assess the presence, expression and/or gene copy number in a given cell, tissue, or organism. These uses may be relevant for diagnosis of disorders involving an increase or decrease in gene expression relative to normal levels. In vitro techniques for detection of mRNA may include Northern blot hybridizations and in situ hybridizations. In vitro techniques for detecting DNA include Southern blot hybridizations and in situ hybridizations.

Probes can be used as part of a diagnostic test kit for identifying cells or tissues in which a variant protein may be expressed, such as by measuring the level of a variant protein-encoding nucleic acid (e.g., mRNA) in a sample of cells from a subject or determining if a polynucleotide contains a SNP of interest.

Thus, the nucleic acid molecules of the disclosure can be used as hybridization probes to detect the SNPs, thereby determining whether an individual with the polymorphisms may be at risk for DDD or has developed early stage DDD. Detection of a SNP associated with a DDD phenotype provides a diagnostic and/or a prognostic tool for an active DDD and/or genetic predisposition to the DDD.

The nucleic acid molecules of the disclosure may be useful as primers to amplify any given region of a nucleic acid molecule, particularly a region containing a SNP.

The nucleic acid molecules of the disclosure may be useful for constructing vectors containing a gene regulatory region of the nucleic acid molecules.

### SNP Genotyping Methods

The process of determining which specific nucleotide (i.e., allele) may be present at each of one or more SNP positions, such as a SNP position in a nucleic acid molecule characterized by a SNP, may be referred to as SNP genotyping. The present disclosure provides methods of SNP genotyping, such as for use in screening for DDD or related pathologies, or determining predisposition thereto, or determining responsiveness to a form of treatment, or in genome mapping or SNP association analysis, etc.

Nucleic acid samples can be genotyped to determine which allele(s) is/are present at any given genetic region (e.g., SNP position) of interest. The neighboring sequence can be used to design SNP detection reagents such as oligonucleotide probes, which may be implemented in a kit format. SNP genotyping methods may include TaqMan assays, molecular beacon assays, nucleic acid arrays, allele-specific primer extension, allele-specific PCR, arrayed primer extension, homogeneous primer extension assays, primer extension with detection by mass spectrometry, mass spectrometry with or with monoisotopic dNTPs (U.S. Pat. No. 6,734,294), pyrosequencing, multiplex primer extension sorted on genetic arrays, ligation with rolling circle amplification, homogeneous ligation, OLA (U.S. Pat. No. 4,988,167), multiplex ligation reaction sorted on genetic arrays, restriction-fragment length polymorphism, single base extension-tag assays, and the Invader assay. Such methods may be used in combination with detection mechanisms such as luminescence or chemiluminescence detection, fluorescence detection, time-resolved fluorescence detection, fluorescence resonance energy transfer, fluorescence polarization, mass spectrometry, electrospray mass spectrometry, and electrical detection.

Various methods for detecting polymorphisms may include methods in which protection from cleavage agents may be used to detect mismatched bases in RNA/RNA or RNA/DNA duplexes, comparison of the electrophoretic mobility of variant and wild type nucleic acid molecules, and assaying the movement of polymorphic or wild-type fragments in polyacrylamide gels containing a gradient of denaturant using denaturing gradient gel electrophoresis (DGGE). Sequence variations at specific locations can also be assessed by nuclease protection assays such as RNase and 51 protection or chemical cleavage methods.

In some embodiments, SNP genotyping is performed using the TaqMan assay. The TaqMan assay may detect the accumulation of a specific amplified product during PCR. The TaqMan assay may utilize an oligonucleotide probe labeled with a fluorescent reporter dye and a quencher dye. The reporter dye may be excited by irradiation at an appropriate wavelength, it transfers energy to the quencher dye in the same probe via a process called fluorescence resonance energy transfer (FRET). When attached to the probe, the excited reporter dye may not emit a signal. In some embodiments, the proximity of the quencher dye to the reporter dye in the intact probe may maintain a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye may be attached to an internal nucleotide, or vice versa. In some embodiments, both the reporter and the quencher may be attached to internal nucleotides at a distance from each other such that fluorescence of the reporter may be reduced.

During PCR, the 5′ nuclease activity of DNA polymerase cleaves the probe, thereby separating the reporter dye and the quencher dye and resulting in increased fluorescence of the reporter. Accumulation of PCR product may be detected directly by monitoring the increase in fluorescence of the reporter dye. The DNA polymerase cleaves the probe between the reporter dye and the quencher dye only if the probe hybridizes to the target SNP-containing template which may be amplified during PCR, and the probe may be designed to hybridize to the target SNP site only if a particular SNP allele may be present.

TaqMan primer and probe sequences can readily be determined using the SNP and associated nucleic acid sequence information. A number of computer programs, such as Primer Express (Applied Biosystems, Foster City, Calif.), can be used to rapidly obtain optimal primer/probe sets. It may be apparent to one of skill in the art that such primers and probes for detecting the SNPs may be useful in diagnostic assays for DDD and related pathologies, and can be readily incorporated into a kit format. The present disclosure also includes modifications of the Taqman assay.

A method for genotyping the SNPs may include the use of two oligonucleotide probes in an OLA (see, e.g., U.S. Pat. No. 4,988,617). In this method, one probe may hybridize to a segment of a target nucleic acid with its 3′ most end aligned with the SNP site. A second probe may hybridize to an adjacent segment of the target nucleic acid molecule directly 3′ to the first probe. The two juxtaposed probes may hybridize to the target nucleic acid molecule, and may be ligated in the presence of a linking agent such as a ligase if there may be greater than about 99% complementarity between the 3′ most nucleotide of the first probe with the SNP site. If there may be a mismatch, ligation may not occur. After the reaction, the ligated probes may be separated from the target nucleic acid molecule, and detected as indicators of the presence of a SNP.

SNPs can also be scored by direct DNA sequencing. A variety of automated sequencing procedures can be utilized, including sequencing by mass spectrometry. The nucleic acid sequences enable one of ordinary skill in the art to readily design sequencing primers for such automated sequencing procedures. Commercial instrumentation, such as the Applied Biosystems 377, 3100, 3700, 3730, and 3730x1 DNA Analyzers (Foster City, Calif.), may be used in the art for automated sequencing.

SNP genotyping can include the steps of collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification may only occur if a particular SNP allele may be present or absent). In some assays, the size of the amplification product may be detected and compared to the length of a control sample; for example, deletions and insertions can be detected by a change in size of the amplified product compared to a normal genotype.

SNP genotyping may be useful for numerous practical applications, as described below. Examples of such applications may include SNP-DDD association analysis, DDD predisposition screening, DDD diagnosis, DDD prognosis, DDD progression monitoring, determining therapeutic strategies based on an individual's genotype, and stratifying a subject population for clinical trials for a treatment such as minimally invasive device for the treatment of DDD.

### Analysis of Genetic Association Between SNPs and Phenotypic Traits

SNP genotyping for DDD diagnosis, DDD predisposition screening, DDD prognosis and DDD treatment and other uses described herein, may rely on initially establishing a genetic association between one or more specific SNPs and the particular phenotypic traits of interest.

In a genetic association study, the cause of interest to be tested may be a certain allele or a SNP or a combination of alleles or a haplotype from several SNPs. Thus, tissue specimens (e.g., saliva) from the sampled individuals may be collected and genomic DNA genotyped for the SNP(s) of interest. In addition to the phenotypic trait of interest, other information such as demographic (e.g., age, gender, ethnicity, etc.), clinical, and environmental information that may influence the outcome of the trait can be collected to further characterize and define the sample set. Specifically, in a DDD genetic association study, information on the number of herniated discs, sciatica episodes, decreased disc height, dark nucleus pulposus and the Schneiderman or Pfirrmann grade which evaluates signal changes within the nucleus pulposus of the intervertebral discs of the lumbar spine may be collected. In some embodiments, these factors may be associated with diseases and/or SNP allele frequencies. There may be gene-environment and/or gene-gene interactions as well. Analysis methods to address gene-environment and gene-gene interactions (for example, the effects of the presence of both susceptibility alleles at two different genes can be greater than the effects of the individual alleles at two genes combined) are discussed below.

After all the relevant phenotypic and genotypic information has been obtained, statistical analyses may be carried out to determine if there may be any significant correlation between the presence of an allele or a genotype with the phenotypic characteristics of an individual. Data inspection and cleaning may be first performed before carrying out statistical tests for genetic association. Epidemiological and clinical data of the samples can be summarized by descriptive statistics with tables and graphs. Data validation may be performed to check for data completion, inconsistent entries, and outliers. Chi-squared tests may then be used to check for significant differences between cases and controls for discrete and continuous variables, respectively. To ensure genotyping quality, Hardy-Weinberg disequilibrium tests can be performed on cases and controls separately. Significant deviation from Hardy-Weinberg equilibrium (HWE) in both cases and controls for individual markers can be indicative of genotyping errors. If HWE may be violated in a majority of markers, it may be indicative of population substructure that may be further investigated. Moreover, Hardy-Weinberg disequilibrium in cases only can indicate genetic association of the markers with the disease of interest. (Genetic Data Analysis, Weir B., Sinauer (1990)).

To test whether an allele of a single SNP may be associated with the case or control status of a phenotypic trait, one skilled in the art can compare allele frequencies in cases and controls. Standard chi-squared tests and Fisher exact tests can be carried out on a 2×2 table (2 SNP alleles×2 outcomes in the categorical trait of interest). To test whether genotypes of a SNP may be associated, chi-squared tests can be carried out on a 3×2 table (3 genotypes×2 outcomes). Score tests may be carried out for genotypic association to contrast the three genotypic frequencies (major homozygotes, heterozygotes and minor homozygotes) in cases and controls, and to look for trends using 3 different modes of inheritance, namely dominant (with contrast coefficients 2, −1, −1), additive (with contrast coefficients 1, 0, −1) and recessive (with contrast coefficients 1, 1, −2). Odds ratios for minor versus major alleles, and odds ratios for heterozygote and homozygote variants versus the wild type genotypes may be calculated with the desired confidence limits, usually 95%.

In order to control for confounding effects and to test for interactions (such as cross-product terms), methods as described herein may include performing a stepwise multiple logistic regression analysis (for example by using any statistical packages such as SAS or R). Logistic regression analysis may comprise a model-building technique in which the best fitting and most parsimonious model may be built to describe the relation between the dichotomous outcome (for instance, getting DDD or not) and a set of independent variables (for instance, genotypes of different associated genes, and the associated demographic and environmental factors). The model may be one in which the logit transformation of the odds ratios may be expressed as a linear combination of the variables (main effects) and their cross-product terms (interactions) (Applied Logistic Regression, Hosmer and Lemeshow, Wiley (2000)). To test whether a certain variable or interaction may be significantly associated with the outcome, coefficients in the model may be first estimated and then tested for statistical significance of their departure from zero.

In addition to performing association tests one marker at a time, haplotype association analysis may also be performed to study a number of markers that may be closely linked together. Haplotype association tests can have better power than genotypic or allelic association tests when the tested markers may not be the disease-causing mutations themselves but may be in linkage disequilibrium with such mutations. The test may even be more powerful if DDD may be indeed caused by a combination of alleles on a haplotype. In order to perform haplotype association effectively, marker-marker linkage disequilibrium measures, both D′ and r2, may be calculated for the markers within a gene to elucidate the haplotype structure. Recent studies in linkage disequilibrium indicate that SNPs within a gene may be organized in block pattern, and a high degree of linkage disequilibrium exists within blocks and very little linkage disequilibrium exists between blocks. Haplotype association with DDD status can be performed using such blocks once they have been elucidated.

Haplotype association tests can be carried out in a similar fashion as the allelic and genotypic association tests. Each haplotype in a gene may be analogous to an allele in a multi-allelic marker. One skilled in the art can either compare the haplotype frequencies in cases and controls or test genetic association with different pairs of haplotypes. It has been proposed that score tests can be done on haplotypes using the program “haplo.score”. In that method, haplotypes may be first inferred by EM algorithm and score tests may be carried out with a generalized linear model (GLM) framework that allows the adjustment of other factors.

An important decision in the performance of genetic association tests may be the determination of the significance level at which significant association can be declared when the p-value of the tests reaches that level. In an exploratory analysis where positive hits may be followed up in subsequent confirmatory testing, an unadjusted p-value <0.1 (a significance level on the lenient side) may be used for generating hypotheses for significant association of a SNP with certain phenotypic characteristics of a DDD. A p-value <0.05 (a significance level traditionally used in the art) may be achieved in order for a SNP to be considered to have an association with DDD. A p-value <0.01 (a significance level on the stringent side) may be achieved for an association to be declared. However, in some embodiments, a SNP having a p-value >0.05 may be declared to have an association for reasons such as having a high diagnostic odds ratio. When hits may be followed up in confirmatory analyses in more samples of the same source or in different samples from different sources, adjustment for multiple testing may be performed as to avoid excess number of hits while maintaining the experiment-wise error rates at 0.05. While there may be different methods to adjust for multiple testing to control for different kinds of error rates, a method may be Bonferroni correction to control the experiment-wise or family-wise error rate. Permutation tests to control for the false discovery rates, FDR, can be more powerful. Such methods to control for multiplicity may be employed when the tests may be dependent and controlling for false discovery rates may be sufficient as opposed to controlling for the experiment-wise error rates.

In replication studies using samples from different populations after statistically significant markers have been identified in the exploratory stage, meta-analyses can then be performed by combining evidence of different studies. If available, association results for the same SNPs can be included in the meta-analyses.

Since both genotyping and DDD status classification can involve errors, sensitivity analyses may be performed to see how odds ratios and p-values may change upon various estimates on genotyping and DDD classification error rates.

Once individual risk factors, genetic or non-genetic, have been found for the predisposition to DDD, the next step may be to set up a classification/prediction scheme to predict the category (for instance, DDD, no DDD, or DDD progression or non-progression) that an individual may be in depending on his genotypes of associated SNPs and other non-genetic risk factors. Logistic regression for discrete trait and linear regression for continuous trait may be standard techniques for such tasks. Moreover, other techniques can also be used for setting up classification. Such techniques may include MART, CART, neural network, and discriminant analyses that may be suitable for use in comparing the performance of different methods.

### DDD Diagnosis and Predisposition Screening

Information on association/correlation between genotypes and DDD-related phenotypes can be exploited in several ways. For example, in the case of a highly statistically significant association between one or more SNPs with predisposition to a disease for which treatment may be available, detection of such a genotype pattern in an individual may justify particular treatment, or at least the institution of regular monitoring of the individual. Detection of the susceptibility alleles associated with a disease in a couple contemplating having children may also be valuable to the couple in their reproductive decisions. In the case of a weaker but still statistically significant association between a SNP and a human disease immediate therapeutic intervention or monitoring may not be justified after detecting the susceptibility allele or SNP.

The SNPs of the disclosure may contribute to DDD in an individual in different ways. Some polymorphisms occur within a protein coding sequence and contribute to DDD phenotype by affecting protein structure. Other polymorphisms occur in noncoding regions but may exert phenotypic effects indirectly via influence on replication, transcription, and/or translation. A single SNP may affect more than one phenotypic trait. Likewise, a single phenotypic trait may be affected by multiple SNPs in different genes.

As used herein, the terms “diagnose”, “diagnosis”, and “diagnostics” may include any of the following: detection of DDD that an individual may presently have or be at risk for, predisposition screening (i.e., determining the increased risk for an individual in developing DDD in the future, or determining whether an individual has a decreased risk of developing DDD in the future;), determining a particular type or subclass of DDD in an individual having DDD, confirming or reinforcing a previously made diagnosis of DDD, predicting the progression of and future prognosis of an individual having DDD. Such diagnostic uses may be based on the SNPs individually or in a unique combination or SNP haplotypes or in combination with SNPs and other non-genetic clinical factors.

Haplotypes may be particularly useful in that fewer SNPs can be genotyped to determine if a particular genomic region harbors a locus that influences a particular phenotype, such as in linkage disequilibrium-based SNP association analysis.

Linkage disequilibrium (LD) refers to the co-inheritance of alleles (e.g., alternative nucleotides) at two or more different SNP sites at frequencies greater than may be expected from the separate frequencies of occurrence of each allele in a given population. The expected frequency of co-occurrence of two alleles that may be inherited independently may be the frequency of the first allele multiplied by the frequency of the second allele. Alleles that co-occur at expected frequencies may be in “linkage equilibrium”. In contrast, LD refers to any non-random genetic association between allele(s) at two or more different SNP sites, which may be generally due to the physical proximity of the two loci along a chromosome. LD can occur when two or more SNPs sites may be in close physical proximity to each other on a given chromosome and therefore alleles at these SNP sites may tend to remain unseparated for multiple generations with the consequence that a particular nucleotide (allele) at one SNP site may show a non-random association with a particular nucleotide (allele) at a different SNP site located nearby. Hence, genotyping one of the SNP sites may give almost the same information as genotyping the other SNP site that may be in LD.

For diagnostic purposes, if a particular SNP site may be found to be useful for diagnosing DDD, then the skilled artisan may recognize that other SNP sites which may be in LD with this SNP site may also be useful for diagnosing the condition. Various degrees of LD can be encountered between two or more SNPs with the result being that some SNPs may be more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome.

For diagnostic applications, polymorphisms (e.g., SNPs and/or haplotypes) that may not be the actual disease-causing (causative) polymorphisms, but may be in LD with such causative polymorphisms may be utilized. In such embodiments, the genotype of the polymorphism(s) that is/are in LD with the causative polymorphism may be predictive of the genotype of the causative polymorphism and, consequently, predictive of the phenotype (e.g., DDD) that may be influenced by the causative SNP(s). Thus, polymorphic markers that may be in LD with causative polymorphisms may be useful as diagnostic markers, and may be particularly useful when the actual causative polymorphism(s) is/are unknown.

In some embodiments, the contribution or association of particular SNPs and/or SNP haplotypes with DDD phenotypes, enables the SNPs to be used to develop superior diagnostic tests capable of identifying individuals who express a detectable trait, such as DDD as the result of a specific genotype, or individuals whose genotype places them at an increased or decreased risk of developing a detectable trait at a subsequent time as compared to individuals who may not have that genotype. As described herein, diagnostics may be based on a single SNP or a group of SNPs. Combined detection of a plurality of SNPs (for example, about: 2, 3, 4, 5, 6, 7, 8, 9, 10, 11, 12, 13, 14, 15, 16, 17, 18, 19, 20, 24, 25, 30, 32, 48, 50, 64, 96, 100, or any other number in-between, or more), of the SNPs provided in FIG. 1 may increase the probability of an accurate diagnosis. For example, the presence of a single SNP correlated with DDD might indicate a odds ratio of 1.5 that an individual has or may be at risk of developing DDD, whereas detection of five SNPs, each of which correlates with DDD, might indicate an odds ratio of 9.5 that an individual has or may be at risk of developing DDD. To further increase the accuracy of diagnosis or predisposition screening, analysis of the SNPs can be combined with that of other polymorphisms or other risk factors of DDD, such as the number of herniated discs, sciatica episodes, decreased disc height, dark nucleus pulposus and the Schneiderman or Pfirrmann grade which evaluates signal changes within the nucleus pulposus of the intervertebral discs of the lumbar spine.

In some embodiments, it may, of course, be understood by practitioners skilled in the treatment or diagnosis of DDD that the present disclosure generally does not intend to provide an absolute identification of individuals who may be at risk (or less at risk) of developing DDD and/or pathologies related to DDD, but rather to indicate a certain increased (or decreased) degree or likelihood of developing the DDD or developing progression of DDD based on statistically significant association results. However, this information may be extremely valuable as it can be used to initiate earlier preventive and/or corrective treatments or to allow an individual carrying one or more significant SNPs or SNP haplotypes to regularly scheduled physical exams to monitor for the appearance or change of their DDD in order to identify and begin treatment of the DDD at an early stage.

The diagnostic techniques may employ a variety of methodologies to determine whether a test subject has a SNP or a SNP pattern associated with an increased or decreased risk of developing a detectable trait or whether the individual suffers from a detectable trait as a result of a particular polymorphism/mutation, including methods which may enable the analysis of individual chromosomes for haplotyping, family studies, single sperm DNA analysis, or somatic hybrids. The trait analyzed using the diagnostics of the disclosure may be any detectable trait that may be observed in pathologies and disorders related to DDD.

Another aspect of the present disclosure relates to a method of determining whether an individual may be at risk (or less at risk) of developing one or more traits or whether an individual expresses one or more traits as a consequence of possessing a particular trait-causing or trait-influencing allele. These methods generally involve obtaining a nucleic acid sample from an individual and assaying the nucleic acid sample to determine which nucleotide(s) is/are present at one or more SNP positions, wherein the assayed nucleotide(s) is/are indicative of an increased or decreased risk of developing the trait or indicative that the individual expresses the trait as a result of possessing a particular trait-causing or trait-influencing allele.

The SNPs can be used to identify novel therapeutic targets for DDD. For example, genes containing the disease-associated variants (“variant genes”) or their products, as well as genes or their products that may be directly or indirectly regulated by or interacting with these variant genes or their products can be targeted for the development of therapeutics that may treat DDD or prevent or delay DDD onset. The therapeutics may be composed of small molecules, proteins, protein fragments or peptides, antibodies, nucleic acids, or their derivatives or mimetics which modulate the functions or levels of the target genes or gene products.

The SNPs/haplotypes may be useful for improving many different aspects of the drug development process. For example, individuals can be selected for clinical trials based on their SNP genotype. Individuals with SNP genotypes that indicate that they may be most likely to respond to or most likely to benefit from a device or a drug can be included in the trials and those individuals whose SNP genotypes indicate that they may be less likely to or may not respond to a device or a drug, or suffer adverse reactions, can be eliminated from the clinical trials. This not only improves the safety of clinical trials, but also may enhance the chances that the trial may demonstrate statistically significant efficacy. Furthermore, the SNPs may explain why certain previously developed devices or drugs performed poorly in clinical trials and may help identify a subset of the population that may benefit from a drug that had previously performed poorly in clinical trials, thereby “rescuing” previously developed devices or drugs, and enabling the device or drug to be made available to a particular DDD subject population that can benefit from it.

### Pharmaceutical Compositions

Any of the DDD-associated proteins, and encoding nucleic acid molecules, can be used as therapeutic targets (or directly used themselves as therapeutic compounds) for treating DDD and related pathologies, and the present disclosure enables therapeutic compounds (e.g., small molecules, antibodies, therapeutic proteins, RNAi and antisense molecules, etc.) to be developed that target (or may be comprised of) any of these therapeutic targets.

### Variant Proteins Encoded by SNP-Containing Nucleic Acid Molecules

The present disclosure provides SNP-containing nucleic acid molecules, many of which encode proteins having variant amino acid sequences as compared to the (i.e., wild-type) proteins. These variants may generally be referred to herein as variant proteins/peptides/polypeptides, or polymorphic proteins/peptides/polypeptides. The terms “protein,” “peptide,” and “polypeptide” are used herein interchangeably.

A variant protein may be encoded by a nonsynonymous nucleotide substitution at any one of the cSNP positions. In addition, variant proteins may also include proteins whose expression, structure, and/or function may be altered by a SNP, such as a SNP that creates or destroys a stop codon, a SNP that affects splicing, and a SNP in control/regulatory elements, e.g. promoters, enhancers, or transcription factor binding domains.

### Uses of Variant Proteins

The variant proteins can be used in a variety of ways that may include in assays to determine the biological activity of a variant protein, such as in a panel of multiple proteins for high-throughput screening; to raise antibodies or to elicit another type of immune response; as a reagent (including the labeled reagent) in assays designed to quantitatively determine levels of the variant protein (or its binding partner) in biological fluids; as a marker for cells or tissues in which it may be preferentially expressed (either constitutively or at a particular stage of tissue differentiation or development or in a DDD state); as a target for screening for a therapeutic agent; and as a direct therapeutic agent to be administered into a human subject. Any of the variant proteins may be developed into reagent grade or kit format for commercialization as research products.

### Computer-Related Embodiments

The SNPs provided in the present disclosure may be “provided” in a variety of mediums to facilitate use thereof. As used in this section, “provided” may refer to a manufacture, other than an isolated nucleic acid molecule, that contains SNP information. Such a manufacture may provide the SNP information in a form that allows a skilled artisan to examine the manufacture using means not directly applicable to examining the SNPs or a subset thereof as they exist in nature or in purified form. The SNP information that may be provided in such a form includes any of the SNP information provided by the present disclosure such as a polymorphic nucleic acid and/or amino acid sequence information of FIG. 1; information about observed SNP alleles, alternative codons, populations, allele frequencies, SNP types, and/or affected proteins; or any other information provided by the present disclosure in FIG. 1 and/or the Sequence Listing.

In some embodiments, the SNPs can be recorded on a computer readable medium. As used herein, “computer readable medium” may refer to any medium that can be read and accessed directly by a computer. Such media may include magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable media can be used to create a manufacture comprising computer readable medium having recorded thereon a nucleotide sequence. One such medium may be provided with the present application, namely, the present application contains computer readable medium (CD-R) that has nucleic acid sequences (and encoded protein sequences) containing SNPs provided/recorded thereon in ASCII text format in a Sequence Listing along with accompanying Tables that contain detailed SNP and sequence information.

As used herein, “recorded” may refer to a process for storing information on computer readable medium. A skilled artisan can readily adopt any of the presently known methods for recording information on computer readable medium to generate manufactures comprising the SNP information.

A variety of data storage structures may be available to a skilled artisan for creating a computer readable medium having recorded thereon a nucleotide or amino acid sequence. The choice of the data storage structure may generally be based on the means chosen to access the stored information. In addition, a variety of data processor programs and formats can be used to store the nucleotide/amino acid sequence information on computer readable medium. For example, the sequence information can be represented in a word processing text file, formatted in commercially-available software such as WordPerfect and Microsoft Word, represented in the form of an ASCII file, or stored in a database application, such as OB2, Sybase, Oracle, or the like. A skilled artisan can readily adapt any number of data processor structuring formats (e.g., text file or database) in order to obtain computer readable medium having recorded thereon the SNP information.

By providing the SNPs in computer readable form, a skilled artisan can access the SNP information for a variety of purposes. Computer software may be publicly available which allows a skilled artisan to access sequence information provided in a computer readable medium. Examples of publicly available computer software include BLAST and BLAZE search algorithms.

The present disclosure further provides systems, particularly computer-based systems, which contain the SNP information described herein. Such systems may be designed to store and/or analyze information on a large number of SNP positions, or information on SNP genotypes from a large number of individuals. The SNP information may represent a valuable information source. The SNP information stored/analyzed in a computer-based system may be used for such computer-intensive applications as determining or analyzing SNP allele frequencies in a population, mapping DDD genes, genotype-phenotype association studies, grouping SNPs into haplotypes, correlating SNP haplotypes with response to particular treatments or for various other bioinformatic, pharmacogenomic or drug development.

As used herein, “a computer-based system” may refer to the hardware means, software means, and data storage means used to analyze the SNP information. The minimum hardware means of the computer-based systems may comprise a central processing unit (CPU), input means, output means, and data storage means. A skilled artisan can readily appreciate that any one of the currently available computer-based systems may be suitable for use in the present disclosure. Such a system can be changed into a system by utilizing the SNP information provided on the CD-R, or a subset thereof, without any experimentation.

As stated above, the computer-based systems may comprise a data storage means having stored therein SNPs and the necessary hardware means and software means for supporting and implementing a search means. As used herein, “data storage means” refers to memory which can store SNP information, or a memory access means which can access manufactures having recorded thereon the SNP information.

As used herein, “search means” may refer to one or more programs or algorithms that may be implemented on the computer-based system to identify or analyze SNPs in a target sequence based on the SNP information stored within the data storage means. Search means can be used to determine which nucleotide may be present at a particular SNP position in the target sequence. As used herein, a “target sequence” can be any DNA sequence containing the SNP position(s) to be searched or queried.

As used herein, “a target structural motif,” or “target motif,” may refer to any rationally selected sequence or combination of sequences containing a SNP position in which the sequence(s) may be chosen based on a three-dimensional configuration that may be formed upon the folding of the target motif. Protein target motifs may include enzymatic active sites and signal sequences. Nucleic acid target motifs may include promoter sequences, hairpin structures, and inducible expression elements (protein binding sequences).

A variety of structural formats for the input and output means can be used to input and output the information in the computer-based systems. An exemplary format for an output means may be a display that depicts the presence or absence of specified nucleotides (alleles) at particular SNP positions of interest. Such presentation can provide a rapid, binary scoring system for many SNPs simultaneously.

### Specific Embodiments

A number of methods and systems are disclosed herein. Specific exemplary embodiments of these methods and systems are disclosed below.

Embodiment 1. A method comprising: detecting one or more single nucleotide polymorphisms (SNPs) in genetic material from a subject having, suspected of having, or developing degenerative disc disease (DDD), wherein said one or more SNPs comprise one or more of SNP 1-SNP 276 of FIG. 1, or any combination thereof.

Embodiment 2. The method of embodiment 1, wherein said one or more SNPs comprise: SNP 6; SNP 76; SNP 111; SNP 212, or any combination thereof.

Embodiment 3. The method of embodiment 1, wherein said one or more SNPs comprise: SNP 25; SNP 88; SNP 120; SNP 188, or any combination thereof.

Embodiment 4. The method of embodiment 1, wherein said one or more SNPs comprise: SNP 1; SNP 6; SNP 13; SNP 55; SNP 65; SNP 76; SNP 85; SNP 86; SNP 100; SNP 109; SNP 111; SNP 132; SNP 145; SNP 154; SNP 211; SNP 212, or any combination thereof.

Embodiment 5. The method of embodiment 1, wherein said one or more SNPs comprise: SNP 1-SNP 4; SNP 6; SNP 7; SNP 10; SNP 11; SNP 13-SNP 15; SNP 17; SNP 19; SNP 20; SNP 22; SNP 23; SNP 25; SNP 28; SNP 29; SNP 35; SNP 37; SNP 40; SNP 41; SNP 44; SNP 50-SNP 56; SNP 60; SNP 62; SNP 65-SNP 67; SNP 69-SNP 72; SNP 74; SNP 76; SNP 79; SNP 80; SNP 82; SNP 84-SNP 87; SNP 92-SNP 103; SNP 105; SNP 106; SNP 108-SNP 111; SNP 114-SNP 116; SNP 119-SNP 121; SNP 124-SNP 127; SNP 129-SNP 133; SNP 136; SNP 138; SNP 141-SNP 145; SNP 147; SNP 148; SNP 150-SNP 154; SNP 156-SNP 161; SNP 163; SNP 165; SNP 166; SNP 168; SNP 171-174; SNP 177; SNP 178; SNP 180; SNP 181; SNP 183-193; SNP 195; SNP 197; SNP 198; SNP 201; SNP 203-207; SNP 209; SNP 211-214; SNP 216-221; SNP 226; SNP 227; SNP 231; SNP 232; SNP 234-239; SNP 241-244; SNP 251; SNP 253; SNP 257-266; SNP 268-SNP 272; SNP 274-SNP 276; or any combination thereof.

Embodiment 6. The method of any one of embodiments 1-5, wherein said one or more SNPs comprise a SNP defining a minor allele.

Embodiment 7. The method of any one of embodiments 1-6, wherein said one or more SNPs comprise at least about: 5, 10, 15, 20, 25, 50, 75, 100, 150, 200, 250, 500, or more SNPs defining minor alleles.

Embodiment 8. The method of any one of embodiments 1-7, wherein detection of said one or more SNPs has an odds ratio (OR) for DDD of at least about: 2 or more.

Embodiment 9. The method of any one of embodiments 1-8, wherein said detecting comprises a high throughput method.

Embodiment 10. The method of any one of embodiments 1-8, wherein said detecting comprises sequencing, hybridization, or nucleic acid amplification.

Embodiment 11. The method of embodiment 10, wherein said detecting comprises sequencing and wherein said sequencing comprises next-gen sequencing.

Embodiment 12. The method of embodiment 10, wherein said detecting comprises sequencing and wherein said sequencing comprises nanopore sequencing.

Embodiment 13. The method of embodiment 12, wherein said nanopore sequencing is performed with a biological nanopore, a solid state nanopore, or a hybrid nanopore.

Embodiment 14. The method of any one of embodiments 1-8, wherein said detecting comprises labeling said one or more SNPs.

Embodiment 15. The method of embodiment 14, wherein said labeling comprises associating a fluorescent label with said one or more SNPs.

Embodiment 16. The method of embodiment 14, wherein said labeling comprises covalently labeling said one or more SNPs.

Embodiment 17. The method of any one of embodiments 1-14, wherein said genetic material comprises RNA.

Embodiment 18. The method of embodiment 17, wherein said RNA comprises mRNA.

Embodiment 19. The method of any one of embodiments 1-16, wherein said genetic material comprises DNA.

Embodiment 20. The method of embodiment 19, wherein said DNA comprises cDNA, genomic DNA, sheared DNA, cell free DNA, fragmented DNA, or PCR amplified products produced therefrom, or any combination thereof.

Embodiment 21. The method of any one of embodiments 1-20, wherein said genetic material is obtained from a biopsy or a fine needle aspirate sample.

Embodiment 22. The method of any one of embodiments 1-20, wherein said genetic material is comprised in blood.

Embodiment 23. The method of any one of embodiments 1-20, wherein said genetic material is comprised in a spinal fluid.

Embodiment 24. The method of any one of embodiments 1-20, wherein said genetic material is comprised in a cell-free sample.

Embodiment 25. The method of any one of embodiments 1-20, wherein said genetic material is comprised in a cell-free DNA.

Embodiment 26. The method of any one of embodiments 1-20, wherein said genetic material is comprised in disc tissue.

Embodiment 27. The method of any one of embodiments 1-26, wherein said detecting yields a data set.

Embodiment 28. The method of any one of embodiments 27, further comprising inputting said data set into a programmed computer having a trained algorithm.

Embodiment 29. The method of embodiment 28, further comprising identifying a risk of said subject having or developing DDD based on a result from said trained algorithm.

Embodiment 30. The method of embodiment 28, further comprising identifying said subject as having DDD based on a result from said trained algorithm.

Embodiment 31. The method of any one of embodiments 29-30, further comprising outputting an electronic report that comprises said result.

Embodiment 32. The method of any one of embodiments 28-31, wherein said trained algorithm compares said data set to a control set.

Embodiment 33. The method of embodiment 32, wherein said control set comprises a sample obtained from a subject positive for DDD.

Embodiment 34. The method of any one of embodiments 28-33, wherein said one or more SNPs are weighted based on (i) a symptom reported by said subject, (ii) a clinical metric obtained from said subject, (iii) a result from said trained algorithm, or (iv) any combination thereof.

Embodiment 35. The method of embodiment 29, wherein said identifying said risk of said subject having or developing DDD is with a specificity of at least: 80%, 85%, 90%, 95%, 96%, 97%, 98%, or 99%.

Embodiment 36. The method of embodiment 29, wherein said identifying said risk of said subject having or developing DDD is with a sensitivity of at least: 80%, 85%, 90%, 95%, 96%, 97%, 98%, or 99%.

Embodiment 37. The method of embodiment 29, wherein said identifying said risk of said subject having or developing DDD is with an accuracy of at least: 80%, 85%, 90%, 95%, 96%, 97%, 98%, or 99%.

Embodiment 38. The method of any one of embodiments 1-37, further comprising administering a therapeutic to said subject.

Embodiment 39. The method of embodiment 38, wherein said therapeutic comprises a regenerative therapy, a medical device, a pharmaceutical composition, a medical procedure, or any combination thereof.

Embodiment 40. The method of embodiment 39, wherein said therapeutic comprises said medical device, and wherein said medical device comprises a spinal brace or an artificial disc device.

Embodiment 41. The method of embodiment 39, wherein said therapeutic comprises said pharmaceutical composition, and wherein said pharmaceutical composition comprises a muscle relaxant, an anti-depressant, a steroid, an opioid, a cannabis-based therapeutic, acetaminophen, a non-steroidal anti-inflammatory, a neuropathic agent, or any combination thereof.

Embodiment 42. The method of embodiment 41, wherein said pharmaceutical composition comprises said neuropathic agent, and wherein said neuropathic agent comprises gabapentin.

Embodiment 43. The method of embodiment 41, wherein said pharmaceutical composition comprises said non-steroidal anti-inflammatory, and wherein said non-steroidal anti-inflammatory comprises naproxen, ibuprofen, a COX-2 inhibitor, or any combination thereof.

Embodiment 44. The method of embodiment 39, wherein said therapeutic comprises said medical procedure, and wherein said medical procedure comprises an epidural injection, a facet joint injection, acupuncture, exercise, physical therapy, spinal surgery, ultrasound, facet rhizotomy, intradiscal electrothermal annuloplasty (IDET), or any combination thereof.

Embodiment 45. The method of embodiment 39, wherein said therapeutic comprises said regenerative therapy, and wherein said regenerative therapy comprises a stem cell, a cord blood cell, an umbilical cord tissue, a tissue, or any combination thereof.

Embodiment 46. The method of embodiment 39, wherein said therapeutic comprises said pharmaceutical composition, and wherein said pharmaceutical composition comprises cannabis.

Embodiment 47. The method of any one of embodiments 1-46, wherein said subject is a human subject.

Embodiment 48. The method of any one of embodiments 1-46, wherein said subject is a canine.

Embodiment 49. The method of embodiment 47, wherein said human subject is a human fetus.

Embodiment 50. The method of any one of embodiments 1-45, wherein said subject has at least one clinical factor.

Embodiment 51. The method of embodiment 50, wherein said at least one clinical factor comprises a presence of a herniated disc; one or more reported sciatica episodes; a decreased disc height; a dark nucleus pulposus; a Schneiderman grade or a Pfirrmann grade showing signal changes within a nucleus pulposus of an intervertebral disc of a lumbar spine; or any combination thereof.

Embodiment 52. The method of any one of embodiments 1-51, wherein said subject is asymptomatic for DDD.

Embodiment 53. The method of any one of embodiments 1-51, wherein said subject is symptomatic for DDD.

Embodiment 54. The method of any one of embodiments 1-53, further comprising administering an imaging procedure to said subject.

Embodiment 55. The method of embodiment 54, wherein said imaging procedure comprises an ultrasound, an x-ray, a magnetic resonance imaging (MRI), a computed tomography (CT) scan, or any combination thereof.

Embodiment 56. A kit comprising: one or more probes for detecting one or more single nucleotide polymorphisms (SNPs) of FIG. 1 in a sample.

Embodiment 57. The kit of embodiment 56, further comprising a control sample.

Embodiment 58. The kit of embodiment 56, wherein said control sample comprises one or more of SNPs of FIG. 1.

Embodiment 59. The kit of embodiment 56, wherein a probe of said one or more probes comprises a sequence having at least 80% sequence complementarity to a sequence adjacent thereto a SNP of said one or more SNPs of FIG. 1.

Embodiment 60. The kit of embodiment 56, wherein said one or more probes comprise a hybridization probe or amplification primer.

Embodiment 61. The kit of embodiment 56, wherein said one or more probes is configured to detect a variant allele in said sample.

Embodiment 62. The kit of embodiment 56, wherein said one or more probes is configured to hybridize to a portion of a nucleic acid of said sample when a variant allele is present in said nucleic acid.

Embodiment 63. The kit of embodiment 56, wherein said one or more probes is configured to associate with a solid support.

Embodiment 64. The kit of embodiment 56, wherein said kit further comprises instructions for use and wherein said instructions for use comprise high stringent hybridization conditions.

Embodiment 65. The kit of embodiment 56, wherein said one or more probes is configured to hybridize to a target region of a nucleic acid of said sample, wherein said target region comprises one or more SNPs.

Embodiment 66. A system comprising: (a) a computer processor configured to receive sequencing data obtained from assaying a sample, wherein said computer processor is configured to identify a presence or an absence of one or more SNPs comprising one or more SNPs of FIG. 1 in said sample, and (b) a graphical user interface configured to display a report comprising said identification of said presence or said absence of said one or more SNPs in said sample.

Embodiment 67. The system of embodiment 66, wherein said computer processor comprises a trained algorithm.

Embodiment 68. The method of any one of embodiments 1-20, wherein said genetic material is comprises of buccal swab or saliva.

Embodiment 69. The method of embodiment 34, wherein said symptom comprises: pain, limitation of mobility, limitation of range of motion, or any combination thereof.

Embodiment 70. The method of embodiment 34, wherein said clinical metric comprises: a blood pressure, a heart rate, a temperature, a weight, a height, an age, a gender, an ethnicity, a medical history, or any combination thereof.

Embodiment 71. The method of embodiment 1, further comprising providing a treatment for said subject, wherein said treatment comprises a recommendation for said treatment.

Embodiment 72. The method of any one of embodiments 1-55, wherein said detecting comprises comparing a data set obtained from said genetic material to a control data set of a control sample.

Embodiment 73. The method of embodiment 72, wherein said data set comprises sequencing data.

Embodiment 74. The method of embodiment 72 or 73, wherein a portion of data from said data set is removed.

Embodiment 75. The method of any one of embodiments 72-74, wherein a portion of data from said control data set is removed.

Embodiment 76. The method of embodiment 74 or 75, wherein an accuracy of said detecting is improved after a removal of said portion of data.

Embodiment 77. The method of embodiment 74 or 75, wherein a false positive rate of said detecting is reduced after a removal of said portion of data.

Embodiment 78. The method of embodiment 75, wherein said portion of data removed from said control data set is data of a sample that is familial to said genetic material.

Embodiment 79. The method of embodiment 72, wherein said control sample is selected based on one or more parameters of associated with said genetic material.

Embodiment 80. The method of embodiment 79, wherein said one or more parameters comprise an ethnicity, an age, a gender, a geographical location, a diet, a medical history, a familial history, a sample preparation, or any combination thereof.

### EXAMPLES

### Example 1

A cell-free sample will be obtained from a human subject at risk of developing DDD. Next generation sequencing will be performed on the cell-free sample to detect a presence or an absence of one or more SNPs of FIG. 1. A report will be generated with a classification of the cell-free sample based on the detected presence or absence of the one or more SNPs of FIG. 1. The classification will confirm whether the subject is at risk of developing DDD.

### Example 2

A blood sample will be obtained from a canine subject symptomatic for DDD. Nanopore sequencing will be performed on a portion of the sample to detect one or more SNPs of FIG. 1. Results of the nanopore sequencing will be input into a trained algorithm. An output from the trained algorithm will identify a stage of DDD of the canine subject.

### Example 3

A subject will complete a medical questionaire. A subject will provide a sample for sequencing analysis. A presence or absence of one or more SNPs of FIG. 1 will be detected in the sample. Results of the medical questionaire and the sequencing analysis will provide a stratified classification of the subject having either a low risk or high risk of developing DDD.

### Example 4

A subject asymptomatic for DDD will provide a sample as part of a screening exam. The sample will be analyzed for a presence or an absence of one or more SNPs of FIG. 1. The results of the analysis will be compared to a reference. Based on a comparison to the result, a subject will receive an indication of risk of developing DDD in the future.

### Example 5

A sample obtained from a subject suspected of having DDD will be assayed for a plurality of SNPs including rs775168347, rs777196456, rs770445456, and rs781899701. A result of the assaying will be input into a trained algorithm. The trained algorithm will output a result including a classification of a presence or an absence of DDD in the sample at an accuracy of at least about 85%.

### Example 6

A sample will be assayed using a plurality of primers. One or more primers of the plurality of primers will comprise about 85% sequence complementarity to at least a portion of SNP 6, SNP 76, SNP 111, and SNP 212. The assaying will identify a presence or an absence of one or more SNPs in the sample.

### Example 7

A trained algorithm will be trained with a training set of samples. The training set of samples will comprise samples obtained from at least one subject confirmed to have DDD. The trained algorithm will utilize feature selection to rank or weight a plurality of SNPs. The ranking or weighting will identify SNPs of the plurality of SNPs to include in a biomarker panel to improve an accuracy of a result (including presence or absence of DDD in a sample) obtained by the trained algorithm.

### Example 8

An independent sample, separate from a training set of samples, will be obtained from a subject in need thereof and will be assayed for a presence of a plurality of SNPs, including a biomarker panel identified using the training set of samples. The biomarker panel will include rs775168347, rs777196456, rs770445456, and rs781899701. A result obtained from the assaying will be input to the trained algorithm. The trained algorithm will identify a presence or an absence of DDD in the independent sample with an accuracy of at least 85%.

### Example 9

Samples were run on a next generation sequencing platform, specifically on an Ion Proton system. Whole Exome sequencing (WES) was performed using Ampliseq sequencing. Samples run on WES were then aligned using a Texas Medication Algorithm Project (TMAP) algorithm and variants were called using a Torrent Variant caller with the default parameter settings as established by the manufacturer.

Samples that fell below the two standard deviation from average counts of the coding variant were eliminated from further analysis due to poor sequencing quality. Those samples eliminated from further analysis, if not removed, may contribute to spurious association results.

Population-based association analysis was performed on samples. Familial samples, if included in the case population, may bias association results. Therefore, Identity By Descent (IBD) analysis was performed to remove any samples that were closely related (pi_hat <0.2).

Variants were annotated to distinguish the type of protein change (i.e synonymous, missense, splicing, stop gain, stop loss, frameshift etc).

Variants may differ significantly across different ethnic groups and thereby influence association results. Hence, it may be paramount to compare the case population (of a particular ethnic composition) against a control group having a similar ethnic composition, such as a reference population. Principal Component Analysis (PCA) was performed to assign various samples of the case population to distinct ethnic groups. In this study, Caucasian or Northern European ancestry was selected as the ethnic group. Association was performed using Caucasian subjects having DDD against a Non-Finnish European cohort obtained from a gnomad database. Samples of the gnomad database were primarily run on an Illumina sequencing platform across different laboratories. In order to eliminate association results potentially influenced by sequencing platform artifacts, the associated results were verified against Caucasian control subjects run using an Ion Proton system.

Homopolymer regions surrounding the variant of interest as well as variants called primarily on unidirectional sequencing strands may also add spurious association. Therefore, associated results were further subjected to visual verification. Visual verification may require each individual variant verified using the bam file on sequence visualization software.

A sample may be compared to a control or reference sample or one or more samples obtained from a reference population. Sequencing data obtained from a sample may be compared to sequencing data obtained from a control or reference sample. A data set obtained from a sample may be compared to a data set obtained from a control or reference sample. A control or reference sample may be selected based on one or more parameters associated with the sample (such as an ethnicity, age, gender, geographical location, diet, medical history, familial history, or others).

Confounding effects may be removed from a data set obtained from a sample, such as sequencing data set. Removal of confounding effects may improve a diagnostic accuracy, sensitivity, specificity, or any combination thereof of a method as described herein. For example, samples having less than about: 5, 4, 3, 2.5, 2, 1.5, 1 standard deviation from average counts of a coding variant may be removed from a data set. Data obtained from samples identified as familial samples relative to the sample of interest may be removed from a data set. A data set may be compared to a reference or control data set having similar ethnicity. Data obtained from homopolymer regions surrounding a variant of interest may be removed from a data set. Data obtained for variants called primarily on unidirectional sequencing strands may be removed from a data set. Any of the forgoing alone or in combination may be confounding effects that may be removed from a data set to yield an improved diagnostic accuracy, sensitivity, specificity, or combination thereof of a method as described herein.

Confounding effects may be removed from a data set prior to a comparison to a control or reference sample Confounding effects may be removed after a comparison. Samples identified as familial samples may be removed prior to obtaining a data set, such as prior to a sequencing.

### Other Examples

A whole-genome case-control approach was used to identify the single nucleotide polymorphisms that are closely associated with the development of DDD and especially significantly symptomatic DDD. Case samples and controls were collected from the same geographical region, were principally Caucasian and generally of Northern and Western European descent. Individuals were determined to have DDD after medical record and MRI and/or X-ray review by at least one orthopedic surgeon. In one example, about 96 DNA samples from DDD subjects and 1504 controls were genotyped using the Affymetrix GeneChip 6.0 SNP microarray system. Controls were defined as individuals from the same geographical region who did not have DDD (e.g. did not have DDD symptoms).

A SNP may be a DNA sequence variation, occurring when a single nucleotide ¬adenine (A), thymine (T), cytosine (C) or guanine (G)—in the genome differs between individuals. A variation may occur in at least 1% of the population to be considered a SNP. Variations that occur in less than 1% of the population are, by definition considered to be mutations whether they cause disease or not. SNPs make up 90% of all human genetic variations, and occur every 100 to 300 bases along the human genome. On average, two of every three SNPs substitute cytosine (C) with thymine (T).

GeneChip microarrays consist of small DNA fragments (referred to as probes), chemically synthesized at specific locations on a coated quartz surface. The precise location where each probe may be synthesized may be called a feature, and millions of features can be contained on one array. The probes which represent a sequence containing a human SNP were selected by Affymetrix based on reliability, sensitivity and specificity. In addition to these criteria, the probes were selected to cover the human genome at approximately equal intervals.

The Affymetrix Genome-Wide Human SNP Array 6.0 uses the whole-genome sampling analysis (WSGA) that has been the hallmark characteristic of all previous Affymetrix mapping arrays. This single array interrogates 906600 SNPs by combining the Nsp I and Sty I PCR fractions prior to the DNA purification step and through a reduction in the absolute number of features associated with each individual SNP on the array. This array also contains 945826 copy number probes designed to interrogate CNVs in the genome. Briefly, 250 ng of genomic DNA was digested with Nsp I and Sty I restriction endonuclease and digested fragments were ligated to their respective adapters. The ligated products were then amplified using the polymerase chain reaction (PCR) to amplify fragments between 250-2000 bp in length. The PCR products were purified and diluted to a standard concentration. Furthermore, the PCR products were then fragmented with a DNase enzyme to approximately 25-150 bp in length. This fragmentation process further reduced the complexity of the genomic sample. Still further, the fragmented PCR products were labeled with a biotin/streptavidin system and allowed to hybridize to the microarray. After hybridization the arrays were stained and non-specific binding was removed through a series of increasingly stringent washes. The genotypes were determined by detection of the label in an Affymetrix GCS 3000 scanner. Finally, genotypes were automatically called using Affymetrix G-type software or using their command line Birdseed algorithm for SNP Array 6.0 available through Affymetrix Power Tools.

For the data to be considered valid for an individual chip, two internal quality control measures were used. Each individual sample may have exceeded an overall call rate of about >86% and the correct gender of the sample needed to be determined as based on the heterozygosity of the X chromosome SNPs. A SNP that did not have at least a 95% call rate across all subjects was eliminated as having possible genotyping errors. SNPs that were monomorphic, having no apparent variation in cases or controls, were also eliminated from analysis. SNPs with a Minor Allele Frequency (MAF) <3% in cases and/or controls and P<0.001 for deviations from Hardy-Weinberg equilibrium (HWE) in cases as well as in controls were eliminated. After removal of these SNPs approximately 492,892 SNPs were available for analysis.

For each SNP, allelic association was tested against disease affection status. In this case P<0.0001 was considered to be significant for each SNP. Markers were also retained that had a P<0.001 if they showed any neighboring support (if there were two or more significant markers (P<0.001) within +/−10 kb of the marker with a P<0.001). Further validation of the significant SNPs was performed by checking their genotype clusters. SNPs whose genotype clusters were of exceptional quality were retained. Genotype Clusters can be visualized using Affymetrix Genotype Console software. Of the SNPs tested, 276 SNPs were determined to be associated with the disease (see FIG. 1).

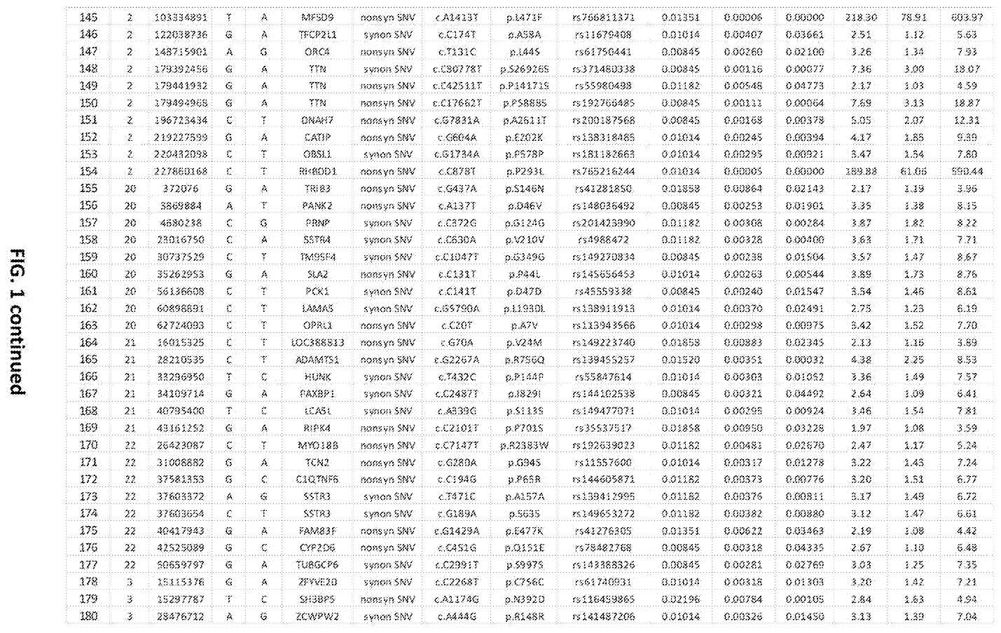

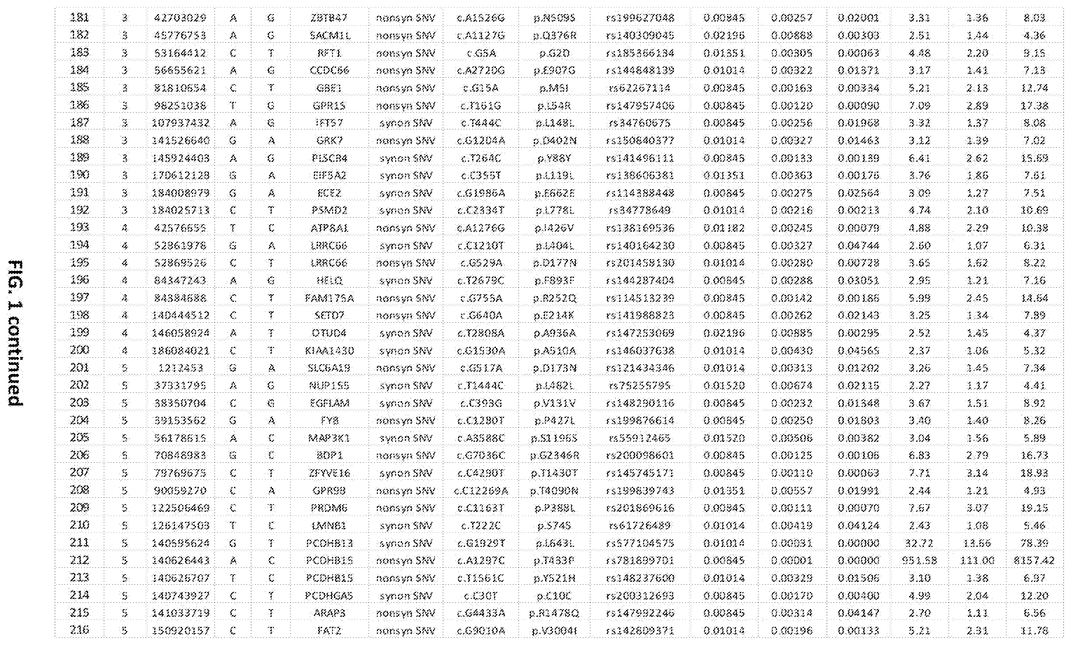

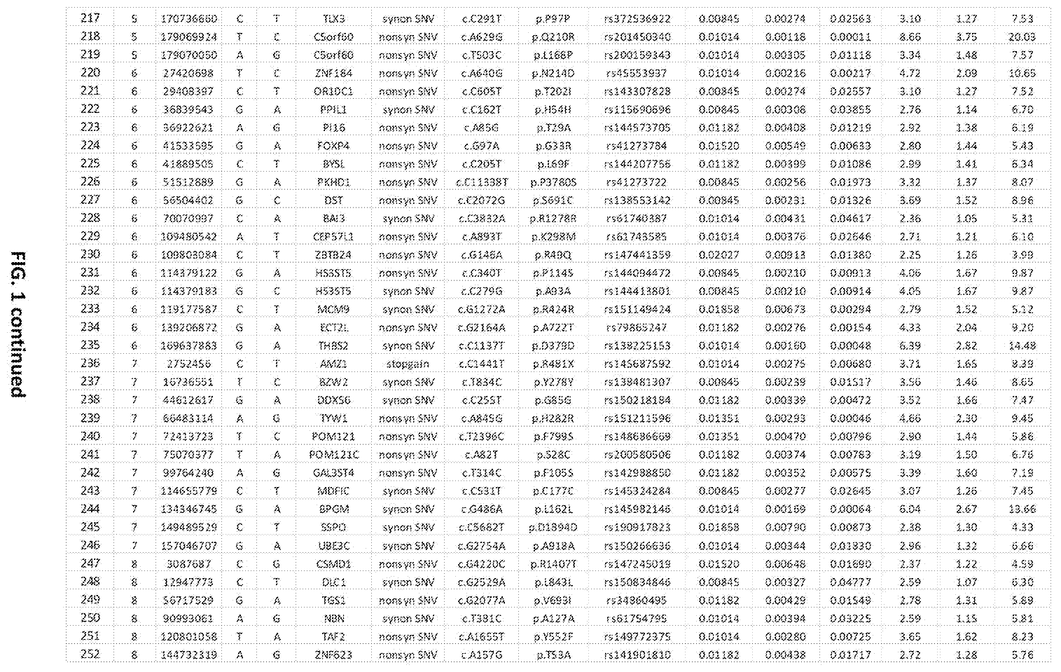

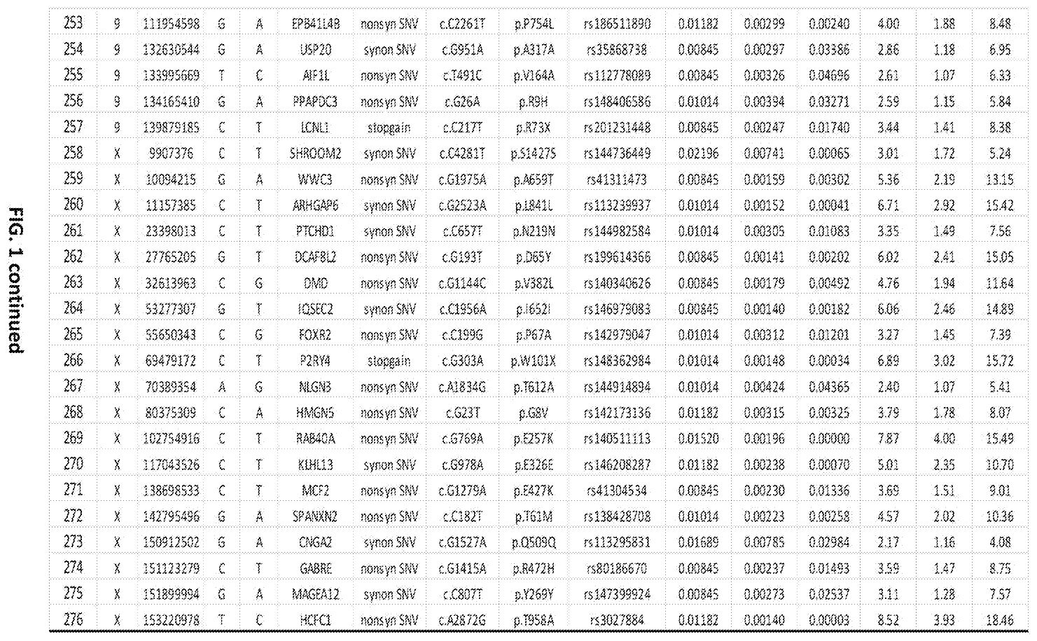

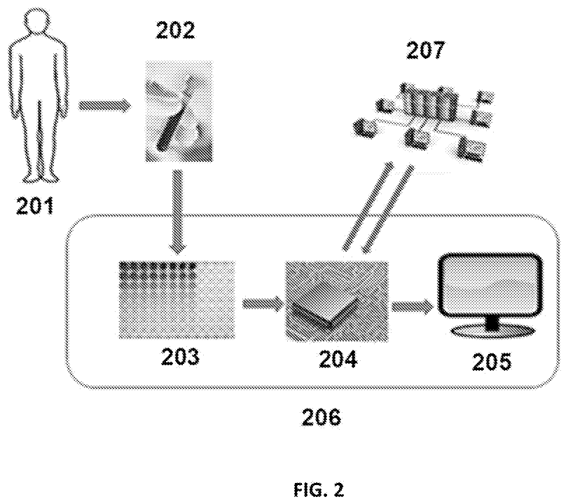

In some embodiments, as shown in FIG. 2, a sample 202 containing a genetic material may be obtained from a subject 201, such as a human subject. A sample 202 may be subjected to one or more methods as described herein, such as performing an assay. In some embodiments, an assay may comprise sequencing (such as nanopore sequencing), genotyping, hybridization, amplification, labeling, or any combination thereof. One or more results from a method may be input into a processor 204. One or more input parameters such as a sample identification, subject identification, sample type, a reference, or other information may be input into a processor 204. One or more metrics from an assay may be input into a processor 204 such that the processor may produce a result, such as a diagnosis of degenerative disc disease or a recommendation for a treatment. A processor may send a result, an input parameter, a metric, a reference, or any combination thereof to a display 205, such as a visual display or graphical user interface. A processor 204 may (i) send a result, an input parameter, a metric, or any combination thereof to a server 207, (ii) receive a result, an input parameter, a metric, or any combination thereof from a server 207, (iii) or a combination thereof.

### Computer Control Systems

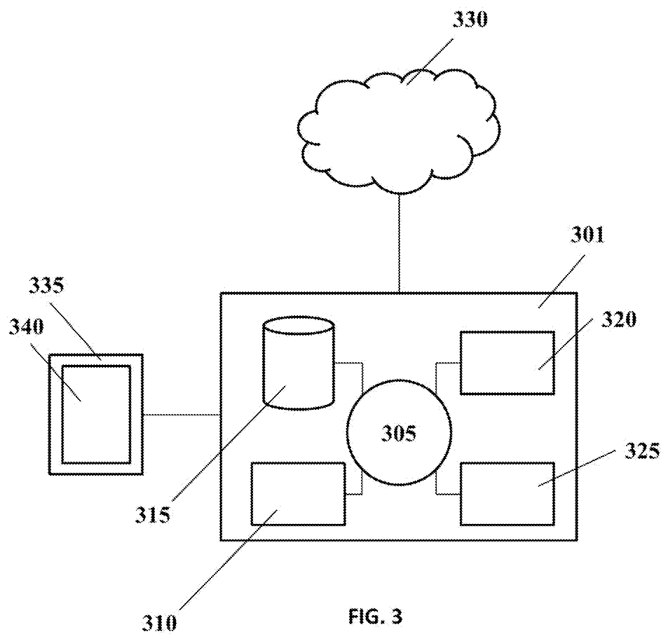

In some embodiments, the present disclosure provides computer control systems that are programmed to implement methods of the disclosure. FIG. 3 shows a computer system 301 that is programmed or otherwise configured to analysis of samples obtained from subject suspected of having or at risk of developing degenerative disc disease (DDD). The computer system 301 can regulate various aspects of analysis and detection of genetic variants (such as SNPs) associated with DDD of the present disclosure. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some embodiments, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some embodiments, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms that may include a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, a graphical user interface or a monitor. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can be for example Polyphen 2, Sift, Mutation Accessor, Mutation Taster, FATHMM, LRT, MetaLR, or any combination thereof. The algorithm can, for example, compare a presence or an absence of an SNP in a test sample with a presence or an absence of the SNP in a control sample.

While some embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it may be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure may also cover any such alternatives, modifications, variations or equivalents. It is intended that the following claims define the scope of the disclosure and that methods and structures within the scope of these claims and their equivalents be covered thereby.

What is claimed is:

1. A method comprising:
detecting one or more single nucleotide polymorphisms (SNPs) in genetic material from a subject having, suspected of having, or developing degenerative disc disease (DDD),
wherein said one or more SNPs comprise one or more of SNP 1-SNP 276 of FIG. 1, or any combination thereof.

2. The method of claim 1, wherein said one or more SNPs comprise: SNP 6; SNP 76; SNP 111; SNP 212, or any combination thereof.

3. The method of claim 1, wherein said one or more SNPs comprise: SNP 25; SNP 88; SNP 120; SNP 188, or any combination thereof.

4. The method of claim 1, wherein said one or more SNPs comprise: SNP 1; SNP 6; SNP 13; SNP 55; SNP 65; SNP 76; SNP 85; SNP 86; SNP 100; SNP 109; SNP 111; SNP 132; SNP 145; SNP 154; SNP 211; SNP 212, or any combination thereof.

5. The method of claim 1, wherein said one or more SNPs comprise: SNP 1-SNP 4; SNP 6; SNP 7; SNP 10; SNP 11; SNP 13-SNP 15; SNP 17; SNP 19; SNP 20; SNP 22; SNP 23; SNP 25; SNP 28; SNP 29; SNP 35; SNP 37; SNP 40; SNP 41; SNP 44; SNP 50-SNP 56; SNP 60; SNP 62; SNP 65-SNP 67; SNP 69-SNP 72; SNP 74; SNP 76; SNP 79; SNP 80; SNP 82; SNP 84-SNP 87; SNP 92-SNP 103; SNP 105; SNP 106; SNP 108-SNP 111; SNP 114-SNP 116; SNP 119-SNP 121; SNP 124-SNP 127; SNP 129-SNP 133; SNP 136; SNP 138; SNP 141-SNP 145; SNP 147; SNP 148; SNP 150-SNP 154; SNP 156-SNP 161; SNP 163; SNP 165; SNP 166; SNP 168; SNP 171-174; SNP 177; SNP 178; SNP 180; SNP 181; SNP 183-193; SNP 195; SNP 197; SNP 198; SNP 201; SNP 203-207; SNP 209; SNP 211-214; SNP 216-221; SNP 226; SNP 227; SNP 231; SNP 232; SNP 234-239; SNP 241-244; SNP 251; SNP 253; SNP 257-266; SNP 268-SNP 272; SNP 274-SNP 276; or any combination thereof.

6. The method of any one of claims 1-5, wherein said one or more SNPs comprise a SNP defining a minor allele.

7. The method of any one of claims 1-6, wherein said one or more SNPs comprise at least about: 5, 10, 15, 20, 25, 50, 75, 100, 150, 200, 250, 500, or more SNPs defining minor alleles.

8. The method of any one of claims 1-7, wherein detection of said one or more SNPs has an odds ratio (OR) for DDD of at least about: 2 or more.

9. The method of any one of claims 1-8, wherein said detecting comprises a high throughput method.

10. The method of any one of claims 1-8, wherein said detecting comprises sequencing, hybridization, nucleic acid amplification, or any combination thereof.

11. The method of claim 10, wherein said detecting comprises sequencing and wherein said sequencing comprises next-gen sequencing.

12. The method of claim 10, wherein said detecting comprises sequencing and wherein said sequencing comprises nanopore sequencing.

13. The method of claim 12, wherein said nanopore sequencing is performed with a biological nanopore, a solid state nanopore, a hybrid nanopore, or any combination thereof.

14. The method of any one of claims 1-8, wherein said detecting comprises labeling said one or more SNPs.

15. The method of claim 14, wherein said labeling comprises associating a fluorescent label with said one or more SNPs.

16. The method of claim 14, wherein said labeling comprises covalently labeling said one or more SNPs.

17. The method of any one of claims 1-14, wherein said genetic material comprises RNA.

18. The method of claim 17, wherein said RNA comprises mRNA.

19. The method of any one of claims 1-16, wherein said genetic material comprises DNA.

20. The method of claim 19, wherein said DNA comprises cDNA, genomic DNA, sheared DNA, cell free DNA, fragmented DNA, or PCR amplified products produced therefrom, or any combination thereof.

21. The method of any one of claims 1-20, wherein said genetic material is obtained from a biopsy or a fine needle aspirate sample.

22. The method of any one of claims 1-20, wherein said genetic material is at least partially isolated from a blood sample.

23. The method of any one of claims 1-20, wherein said genetic material is at least partially isolated from a spinal fluid.

24. The method of any one of claims 1-20, wherein said genetic material is at least partially isolated from a cell-free sample.

25. The method of any one of claims 1-20, wherein said genetic material is comprised in a cell-free DNA.

26. The method of any one of claims 1-20, wherein said genetic material is at least partially isolated from a disc tissue.

27. The method of any one of claims 1-26, wherein said detecting yields a data set.

28. The method of any one of claims 27, further comprising inputting said data set into a programmed computer having a trained algorithm.

29. The method of claim 28, further comprising identifying a risk of said subject having or developing DDD based on a result from said trained algorithm.

30. The method of claim 28, further comprising identifying said subject as having DDD based on a result from said trained algorithm.

31. The method of any one of claims 29-30, further comprising outputting an electronic report that comprises said result.

32. The method of any one of claims 28-31, wherein said trained algorithm compares said data set to a control set.

33. The method of claim 32, wherein said control set comprises a sample obtained from a subject positive for DDD.

34. The method of any one of claims 28-33, wherein said one or more SNPs are weighted based on (i) a symptom reported by said subject, (ii) a clinical metric obtained from said subject, (iii) a result from said trained algorithm, or (iv) any combination thereof.

35. The method of claim 29, wherein said identifying said risk of said subject having or developing DDD is with a specificity of at least: 80%, 85%, 90%, 95%, 96%, 97%, 98%, or 99%.

36. The method of claim 29, wherein said identifying said risk of said subject having or developing DDD is with a sensitivity of at least: 80%, 85%, 90%, 95%, 96%, 97%, 98%, or 99%.

37. The method of claim 29, wherein said identifying said risk of said subject having or developing DDD is with an accuracy of at least: 80%, 85%, 90%, 95%, 96%, 97%, 98%, or 99%.

38. The method of any one of claims 1-37, further comprising administering a therapeutic to said subject.

39. The method of claim 38, wherein said therapeutic comprises a regenerative therapy, a medical device, a pharmaceutical composition, a medical procedure, or any combination thereof.

40. The method of claim 39, wherein said therapeutic comprises said medical device, and wherein said medical device comprises a spinal brace or an artificial disc device.

41. The method of claim 39, wherein said therapeutic comprises said pharmaceutical composition, and wherein said pharmaceutical composition comprises a muscle relaxant, an anti-depressant, a steroid, an opioid, at least partially hemp-derived therapeutic, at least partially cannabis-derived therapeutic, a cannabidiol (CBD) oil derived therapeutic, acetaminophen, a non-steroidal anti-inflammatory, a neuropathic agent, or any combination thereof.

42. The method of claim 41, wherein said pharmaceutical composition comprises said neuropathic agent, and wherein said neuropathic agent comprises gabapentin or a salt thereof.

43. The method of claim 41, wherein said pharmaceutical composition comprises said non-steroidal anti-inflammatory, and wherein said non-steroidal anti-inflammatory comprises naproxen, ibuprofen, a COX-2 inhibitor, a salt of any of these, or any combination thereof.

44. The method of claim 39, wherein said therapeutic comprises said medical procedure, and wherein said medical procedure comprises an epidural injection, a facet joint injection, acupuncture, exercise, physical therapy, spinal surgery, ultrasound, facet rhizotomy, intradiscal electrothermal annuloplasty (IDET), or any combination thereof.

45. The method of claim 39, wherein said therapeutic comprises said regenerative therapy, and wherein said regenerative therapy comprises a stem cell, a cord blood cell, an umbilical cord tissue, a tissue, or any combination thereof.

46. The method of claim 39, wherein said therapeutic comprises said pharmaceutical composition, and wherein said pharmaceutical composition comprises cannabis, cannabidiol oil, hemp, or any combination thereof.

47. The method of any one of claims 1-46, wherein said subject is a human subject.

48. The method of any one of claims 1-46, wherein said subject is a canine.

49. The method of claim 47, wherein said human subject is a human fetus.

50. The method of any one of claims 1-45, wherein said subject has at least one clinical factor.

51. The method of claim 50, wherein said at least one clinical factor comprises a presence of a herniated disc; one or more reported sciatica episodes; a decreased disc height; a dark nucleus pulposus; a Schneiderman grade or a Pfirrmann grade showing signal changes within a nucleus pulposus of an intervertebral disc of a lumbar spine; or any combination thereof.

52. The method of any one of claims 1-51, wherein said subject is asymptomatic for DDD.

53. The method of any one of claims 1-51, wherein said subject is symptomatic for DDD.

54. The method of any one of claims 1-53, further comprising administering an imaging procedure to said subject.

55. The method of claim 54, wherein said imaging procedure comprises an ultrasound, an x-ray, a magnetic resonance imaging (MRI), a computed tomography (CT) scan, or any combination thereof.

56. A kit comprising: one or more probes for detecting one or more single nucleotide polymorphisms (SNPs) of FIG. 1 in a sample.

57. The kit of claim 56, further comprising a control sample.

58. The kit of claim 56, wherein said control sample comprises one or more of SNPs of FIG. 1.

59. The kit of claim 56, wherein a probe of said one or more probes comprises a sequence having at least 80% sequence complementarity to a sequence adjacent thereto a SNP of said one or more SNPs of FIG. 1.

60. The kit of claim 56, wherein said one or more probes comprise a hybridization probe or amplification primer.

61. The kit of claim 56, wherein said one or more probes is configured to detect a variant allele in said sample.

62. The kit of claim 56, wherein said one or more probes is configured to hybridize to a portion of a nucleic acid of said sample when a variant allele is present in said nucleic acid.

63. The kit of claim 56, wherein said one or more probes is configured to associate with a solid support.

64. The kit of claim 56, wherein said kit further comprises instructions for use and wherein said instructions for use comprise high stringent hybridization conditions.

65. The kit of claim 56, wherein said one or more probes is configured to hybridize to a target region of a nucleic acid of said sample, wherein said target region comprises one or more SNPs.

66. A system comprising: (a) a computer processor configured to receive sequencing data obtained from assaying a sample, wherein said computer processor is configured to identify a presence or an absence of one or more SNPs comprising one or more SNPs of FIG. 1 in said sample, and (b) a graphical user interface configured to display a report comprising said identification of said presence or said absence of said one or more SNPs in said sample.

67. The system of claim 66, wherein said computer processor comprises a trained algorithm.

68. The method of any one of claims 1-20, wherein said genetic material is comprises of buccal swab or saliva.

69. The method of claim 34, wherein said symptom comprises: pain, limitation of mobility, limitation of range of motion, or any combination thereof.

70. The method of claim 34, wherein said clinical metric comprises: a blood pressure, a heart rate, a temperature, a weight, a height, an age, a gender, an ethnicity, a medical history, or any combination thereof.

71. The method of claim 1, further comprising providing a treatment for said subject, wherein said treatment comprises a recommendation for said treatment.

72. The method of any one of claims 1-55, wherein said detecting comprises comparing a data set obtained from said genetic material to a control data set of a control sample.

73. The method of claim 72, wherein said data set comprises sequencing data.

74. The method of claim 72 or 73, wherein a portion of data from said data set is removed.

75. The method of any one of claims 72-74, wherein a portion of data from said control data set is removed.

76. The method of claim 74 or 75, wherein an accuracy of said detecting is improved after a removal of said portion of data.

77. The method of claim 74 or 75, wherein a false positive rate of said detecting is reduced after a removal of said portion of data.

78. The method of claim 75, wherein said portion of data removed from said control data set is data of a sample that is familial to said genetic material.

79. The method of claim 72, wherein said control sample is selected based on one or more parameters of associated with said genetic material.

80. The method of claim 79, wherein said one or more parameters comprise an ethnicity, an age, a gender, a geographical location, a diet, a medical history, a familial history, a sample preparation, or any combination thereof.

81. The method of claim 39, wherein said therapeutic comprises said pharmaceutical composition, and wherein said pharmaceutical composition is formulated in a unit dose.

82. The system of claim 66, wherein said computer processor communicates a result.

83. The system of claim 82, wherein said result comprises an identification of said presence or said absence of one or more SNPs in said sample.
